SEQUENCE LISTING (191 sequences)

SEQ ID NO: 1 - protein, 50 aa, Artificial Sequence
  Peptide
MGRKKRRQRRRGHSGRKKRRQRRRGHIYPYDVPDYAGDPGRKKRRQRRRG

SEQ ID NO: 2 - protein, 54 aa, Artificial Sequence
  Peptide
GYAAARRRRRRRGHSGYAAARRRRRRRGHIYPYDVPDYAGDPGYAAARRRRRRR

SEQ ID NO: 3 - protein, 54 aa, Artificial Sequence; identical to SEQ ID NO: 2
  Peptide

SEQ ID NO: 4 - protein, 43 aa, Artificial Sequence
  Peptide
GYAAARRRRRRRGHSGYAAARRRRRRRGSGSGYAAARRRRRRR

SEQ ID NO: 5 - protein, 54 aa, Artificial Sequence
  Peptide
GYARRRRRRRRRGHSGYARRRRRRRRRGHIYPYDVPDYAGDPGYARRRRRRRRR

SEQ ID NO: 6 - protein, 43 aa, Artificial Sequence
  Peptide
GYARRRRRRRRRGHSGYARRRRRRRRRGSGSGYARRRRRRRRR

SEQ ID NO: 7 - protein, 7 aa, Artificial Sequence
  Peptide
RRRRRRR

SEQ ID NO: 8 - protein, 13 aa, Artificial Sequence
  Peptide
YARRKKARRQARR

SEQ ID NO: 9 - protein, 11 aa, Artificial Sequence
  Peptide
YGRKKRRQRRR

SEQ ID NO: 10 - protein, 17 aa, Artificial Sequence
  Peptide
AKIWFQNRRMKWKKENN

SEQ ID NO: 11 - protein, 34 aa, Artificial Sequence
  Peptide
DAATATRGRSAASRPTERPRAPARSASRPRRPVE

SEQ ID NO: 12 - protein, 16 aa, Artificial Sequence
  Peptide
RQIKIWFQNRRMKWKK

SEQ ID NO: 13 - protein, 21 aa, Artificial Sequence
  Peptide
TRSSRAGLQFPVGRVHRLLRK

SEQ ID NO: 14 - protein, 26 aa, Artificial Sequence
  Peptide
GWTLNSAGYLLGKINKALAALAKKIL

SEQ ID NO: 15 - protein, 18 aa, Artificial Sequence
  Peptide
KLALKLALKALKAALKLA

SEQ ID NO: 16 - protein, 28 aa, Artificial Sequence
  Peptide
MANLGYWLLALFVTMWTDVGLCKKRPKP

SEQ ID NO: 17 - protein, 18 aa, Artificial Sequence
  Peptide
LLIILRRRIRKQAHAHSK

SEQ ID NO: 18 - protein, 21 aa, Artificial Sequence
  Peptide
KETWWETWWTEWSQPKKKRKV

SEQ ID NO: 19 - protein, 18 aa, Artificial Sequence
  Peptide
RGGRLSYSRRRFSTSTGR

SEQ ID NO: 20 - protein, 15 aa, Artificial Sequence
  Peptide
SDLWEMMMVSLACQY

SEQ ID NO: 21 - protein, 12 aa, Artificial Sequence
  Peptide
TSPLNIHNGQKL

SEQ ID NO: 22 - protein, 7 aa, Artificial Sequence
  Peptide
KRRQRRR

SEQ ID NO: 23 - protein, 7 aa, Artificial Sequence
  Peptide
RKKRRQR

SEQ ID NO: 24 - protein, 8 aa, Artificial Sequence
  Peptide
RKKRRQRR

SEQ ID NO: 25 - protein, 13 aa, Artificial Sequence
  Peptide
GRKKRRQRRRPPQ

SEQ ID NO: 26 - protein, 17 aa, Artificial Sequence
  Peptide
TRQARRNRRRRWRERQR

SEQ ID NO: 27 - protein, 13 aa, Artificial Sequence
  Peptide
TRRQRTRRARRNR

SEQ ID NO: 28 - protein, 16 aa, Artificial Sequence
  Peptide
TRRNKRNRIQEQLNRK

SEQ ID NO: 29 - protein, 18 aa, Artificial Sequence
  Peptide
TAKTRYKAEEAELIAERR

SEQ ID NO: 30 - protein, 22 aa, Artificial Sequence
  Peptide
MDAQTRRRERRAEKQAQWKAAN

SEQ ID NO: 31 - protein, 15 aa, Artificial Sequence
  Peptide
RRRRNRTRRNRRRVR

SEQ ID NO: 32 - protein, 19 aa, Artificial Sequence
  Peptide
KMTRAQRRAAARRNRWTAR

SEQ ID NO: 33 - protein, 13 aa, Artificial Sequence
  Peptide
TRRQRTRRARRNR

SEQ ID NO: 34 - protein, 17 aa, Artificial Sequence
  Peptide
TRQARRNRRRRWRERQR

SEQ ID NO: 35 - protein, 13 aa, Artificial Sequence
  Peptide
GRKKRRQRRRPPQ

SEQ ID NO: 36 - protein, 9 aa, Artificial Sequence
  Peptide
RRRQRRKKR

SEQ ID NO: 37 - protein, 11 aa, Artificial Sequence
  Peptide
AGRKKRRQRRR

SEQ ID NO: 38 - protein, 11 aa, Artificial Sequence
  Peptide
YARKARRQARR

SEQ ID NO: 39 - protein, 11 aa, Artificial Sequence
  Peptide
YARAAARQARA

SEQ ID NO: 40 - protein, 11 aa, Artificial Sequence
  Peptide
YARAARRAARR

SEQ ID NO: 41 - protein, 11 aa, Artificial Sequence
  Peptide
YARAARRAARA

SEQ ID NO: 42 - protein, 11 aa, Artificial Sequence
  Peptide
YARRRRRRRRR

SEQ ID NO: 43 - protein, 11 aa, Artificial Sequence
  Peptide
YAAARRRRRRR

SEQ ID NO: 44 - protein, 7 aa, Artificial Sequence
  Peptide
KKRPKPG

SEQ ID NO: 45 - protein, 15 aa, Artificial Sequence
  Peptide
KRPAATKKAGQAKKL

SEQ ID NO: 46 - protein, 7 aa, Artificial Sequence
  Peptide
PKKKRKV

SEQ ID NO: 47 - protein, 35 aa, Artificial Sequence
  Peptide
GGGARKKAAKAARKKAAKAARKKAAKAARKKAAKA

SEQ ID NO: 48 - protein, 10 aa, Artificial Sequence
  Peptide
KKKKKKKGGC

SEQ ID NO: 49 - protein, 16 aa, Artificial Sequence
  Peptide
RQIKIWFQNRRMKWKK

SEQ ID NO: 50 - protein, 28 aa, Artificial Sequence
  Peptide
GWTLNSAGYLLGKINLKALAALAKISIL

SEQ ID NO: 51 - protein, 21 aa, Artificial Sequence
  Peptide
AGYLLGKINLKALAALAKKIL

SEQ ID NO: 52 - protein, 34 aa, Artificial Sequence
  Peptide
DAATATRGRSAASRPTERPRAPARSASRPRRPVD

SEQ ID NO: 53 - protein, 15 aa, Artificial Sequence
  Peptide
GCRKKRRQRRRPPQC

SEQ ID NO: 54 - protein, 17 aa, Artificial Sequence
  Peptide
TRQARRNRRRRWRERQR

SEQ ID NO: 55 - protein, 15 aa, Artificial Sequence
  Peptide
RRRRNRTRRNRRRVR

SEQ ID NO: 56 - protein, 25 aa, Artificial Sequence
  Peptide
RRRRRRRXXXXXXXXXXXXXXXXGC

SEQ ID NO: 57 - protein, 20 aa, Artificial Sequence
  Peptide
GLFEAIAGFIENGWEGMIDG

SEQ ID NO: 58 - protein, 21 aa, Artificial Sequence
  Peptide
GLFKAIAKFIKGGAWKGLIKG

SEQ ID NO: 59 - protein, 20 aa, Artificial Sequence
  Peptide
GLFKAIAEFIEGGWEGLIEG

SEQ ID NO: 60 - protein, 22 aa, Artificial Sequence
  Peptide
GLFKAIAEFIEGGWEGLIEGCA

SEQ ID NO: 61 - protein, 23 aa, Artificial Sequence
  Peptide
GLFKAIAEFIEGGWEGLIEGWYG

SEQ ID NO: 62 - protein, 23 aa, Artificial Sequence
  Peptide
GLFHAIAHFIHGGWHGLIHGWYG

SEQ ID NO: 63 - protein, 34 aa, Artificial Sequence
  Peptide
WEAALAEALAEALAEALAEHLAEALAEALEALAA

SEQ ID NO: 64 - protein, 30 aa, Artificial Sequence
  Peptide
WEAKLAKALAKALAKHLAKALAKALKACEA

SEQ ID NO: 65 - protein, 26 aa, Artificial Sequence
  Peptide
KKALLALALHHLAHLALHLALALKKA

SEQ ID NO: 66 - protein, 26 aa, Artificial Sequence
  Peptide
KKALLAHALHLLALLALHLAHALKKA

SEQ ID NO: 67 - protein, 27 aa, Artificial Sequence
  Peptide
KKALLALHALHHLALLAHHLAHALKKA

SEQ ID NO: 68 - protein, 29 aa, Artificial Sequence
  Peptide
KKHLLAHALHLLALLALHLAHALAHLKKA

SEQ ID NO: 69 - protein, 20 aa, Artificial Sequence
  Peptide
GLFEALLELLESLWELLLEA

SEQ ID NO: 70 - protein, 20 aa, Artificial Sequence
  Peptide
GLFKALLKLLKSLTKLLLKA

SEQ ID NO: 71 - protein, 20 aa, Artificial Sequence
  Peptide
GLFRALLRLLRSLTALLLRA

SEQ ID NO: 72 - protein, 12 aa, Artificial Sequence
  Peptide
PKKKRKVEDPYC

SEQ ID NO: 73 - protein, 27 aa, Artificial Sequence
  Peptide
SSDDEATADSQHSTPPKKKRKVEDPYC

SEQ ID NO: 74 - protein, 19 aa, Artificial Sequence
  Peptide
MRRAHHRRRRASHRRMRGG

SEQ ID NO: 75 - protein, 27 aa, Artificial Sequence
  Peptide
GALFLGFLGAAGSTMGAWSQPKSKRKV

SEQ ID NO: 76 - protein, 31 aa, Artificial Sequence
  Peptide
MPKTRRRPRRSQRKRPPTWAHFPGFGQGSLC

SEQ ID NO: 77 - protein, 26 aa, Artificial Sequence
  Peptide
GIGAVLKVLTTGLPALISWIKRKRQQ

SEQ ID NO: 78 - protein, 27 aa, Artificial Sequence
  Peptide
CLIKKALAALAKLNIKLLYGASNLTWG

SEQ ID NO: 79 - protein, 27 aa, Artificial Sequence
  Peptide
GALFLGFLGAAGSTMGAWSQPKKKRKV

SEQ ID NO: 80 - protein, 27 aa, Artificial Sequence
  Peptide
GALFLAFLAAALSLMGLWSQPKKKRKV

SEQ ID NO: 81 - protein, 36 aa, Artificial Sequence
  Peptide
GLFGAIAGFIENGWEGMIDGRQIKIWFQNRRMKWKK

SEQ ID NO: 82 - protein, 30 aa, Artificial Sequence
  Peptide
MVKSKIGSWILVLFVAMWSDVGLCKKRPKP

SEQ ID NO: 83 - protein, 23 aa, Artificial Sequence
  Peptide
LIRLWSHLIHIWFQNRRLKWKKK

SEQ ID NO: 84 - protein, 37 aa, Artificial Sequence
  Peptide
LLGDFFRKSKEKIGKEFKRIVQRIKDFLRNLVPRTES

SEQ ID NO: 85 - protein, 31 aa, Artificial Sequence
  Peptide
SWLSKTAKKLENSAKKRISEGIAIAIQGGPR

SEQ ID NO: 86 - protein, 30 aa, Artificial Sequence
  Peptide
ACYCRIPACIAGERRYGTCIYQGRLWAFCC

SEQ ID NO: 87 - protein, 24 aa, Artificial Sequence
  Peptide
FITKALGISYGRKKRRQRRRPPQC

SEQ ID NO: 88 - protein, 19 aa, Artificial Sequence
  Peptide
LGISYGRKKRRQRRRPPQC

SEQ ID NO: 89 - protein, 16 aa, Artificial Sequence
  Peptide
KKWKMRRNQFWIKIQR

SEQ ID NO: 90 - protein, 16 aa, Artificial Sequence
  Peptide
RQIKIWFQNRRMKWKK

SEQ ID NO: 91 - protein, 16 aa, Artificial Sequence
  Peptide
RQIKIWFPNRRMKWKK

SEQ ID NO: 92 - protein, 16 aa, Artificial Sequence
  Peptide
RQPKIWFPNRRMPWKK

SEQ ID NO: 93 - protein, 16 aa, Artificial Sequence
  Peptide
RQIKIWFQNMRRKWKK

SEQ ID NO: 94 - protein, 16 aa, Artificial Sequence
  Peptide
RQIRIWFQNRRMRWRR

SEQ ID NO: 95 - protein, 16 aa, Artificial Sequence
  Peptide
RRWRRWWRRWWRRWRR

SEQ ID NO: 96 - protein, 14 aa, Artificial Sequence
  Peptide
IKIWFQNRRMKWKK

SEQ ID NO: 97 - protein, 7 aa, Artificial Sequence
  Peptide
RRMKWKK

SEQ ID NO: 98 - protein, 11 aa, Artificial Sequence
  Peptide
WFQNRRMKWKK

SEQ ID NO: 99 - protein, 12 aa, Artificial Sequence
  Peptide
IWFQNRRMKWKK

SEQ ID NO: 100 - protein, 13 aa, Artificial Sequence
  Peptide
KIWFQNRRMKWKK

SEQ ID NO: 101 - protein, 15 aa, Artificial Sequence
  Peptide
RQIKIWFQNRRMKWK

SEQ ID NO: 102 - protein, 12 aa, Artificial Sequence
  Peptide
RQIKIWFQNRRM

SEQ ID NO: 103 - protein, 16 aa, Artificial Sequence
  Peptide
RQIKIFFQNRRMKFKK

SEQ ID NO: 104 - protein, 15 aa, Artificial Sequence
  Peptide
TERQIKIWFQNRRMK

SEQ ID NO: 105 - protein, 10 aa, Artificial Sequence
  Peptide
KIWFQNRRMK

SEQ ID NO: 106 - protein, 26 aa, Artificial Sequence
  Peptide
GWTLNSAGYLLGPINKALAALAKKIL

SEQ ID NO: 107 - protein, 18 aa, Artificial Sequence
  Peptide
KLALKLALKAWKAALKLA

SEQ ID NO: 108 - protein, 18 aa, Artificial Sequence
  Peptide
KLALKAALKAWKAAAKLA

SEQ ID NO: 109 - protein, 18 aa, Artificial Sequence
  Peptide
KLALKAAAKAWKAAAKAA

SEQ ID NO: 110 - protein, 18 aa, Artificial Sequence
  Peptide
KITLKLAIKAWKLALKAA

SEQ ID NO: 111 - protein, 18 aa, Artificial Sequence
  Peptide
KIAAKSIAKIWKSILKIA

SEQ ID NO: 112 - protein, 18 aa, Artificial Sequence
  Peptide
KALAKALAKLWKALAKAA

SEQ ID NO: 113 - protein, 18 aa, Artificial Sequence
  Peptide
KLALKLALKWAKLALKAA

SEQ ID NO: 114 - protein, 18 aa, Artificial Sequence
  Peptide
KLLAKAAKKWLLLALKAA

SEQ ID NO: 115 - protein, 18 aa, Artificial Sequence
  Peptide
KLLAKAALKWLLKALKAA

SEQ ID NO: 116 - protein, 18 aa, Artificial Sequence
  Peptide
KALKKLLAKWLAAAKALL

SEQ ID NO: 117 - protein, 18 aa, Artificial Sequence
  Peptide
KLAAALLKKWKKLAAALL

SEQ ID NO: 118 - protein, 18 aa, Artificial Sequence
  Peptide
KALAALLKKWAKLLAALK

SEQ ID NO: 119 - protein, 18 aa, Artificial Sequence
  Peptide
ALALQLALQALQAALQLA

SEQ ID NO: 120 - protein, 18 aa, Artificial Sequence
  Peptide
ELALELALEALEAALELA

SEQ ID NO: 121 - protein, 7 aa, Artificial Sequence
  Peptide
NKIPIKD

SEQ ID NO: 122 - protein, 27 aa, Artificial Sequence
  Peptide
CGPGSDDEAAADAQHAAPPKKKRKVGY

SEQ ID NO: 123 - protein, 17 aa, Artificial Sequence
  Peptide
KRPAATKKAGQAKKKKL

SEQ ID NO: 124 - protein, 8 aa, Artificial Sequence
  Peptide
RRNRRRRW

SEQ ID NO: 125 - protein, 10 aa, Artificial Sequence
  Peptide
APKRKSGVSK

SEQ ID NO: 126 - protein, 10 aa, Artificial Sequence
  Peptide
VQRKRQKLMP

SEQ ID NO: 127 - protein, 42 aa, Artificial Sequence
  Peptide
DTWTGVEALIRILQQLLFIHFRIGCRHSRIGIIQQRRTRNGA

SEQ ID NO: 128 - protein, 26 aa, Artificial Sequence
  Peptide
MGLGLHLLVLAAALQGAWSQPKKKRK

SEQ ID NO: 129 - protein, 26 aa, Artificial Sequence
  Peptide
AAVALLPAVLLALLAPVQRKRQKLMP

SEQ ID NO: 130 - protein, 17 aa, Artificial Sequence
  Peptide
NAKTRRHERRRKLAIER

SEQ ID NO: 131 - protein, 19 aa, Artificial Sequence
  Peptide
KLTRAQRRAAARKNKRNTR

SEQ ID NO: 132 - protein, 18 aa, Artificial Sequence
  Peptide
TAKTRYKARRAELIAERR

SEQ ID NO: 133 - protein, 16 aa, Artificial Sequence
  Peptide
RVIRVWFQNKRCKDKK

SEQ ID NO: 134 - protein, 18 aa, Artificial Sequence
  Peptide
LLGKINLKALAALAKKIL

SEQ ID NO: 135 - protein, 26 aa, Artificial Sequence
  Peptide
GIGAVLKVLTTGLPALISWIKRKRQQ

SEQ ID NO: 136 - protein, 19 aa, Artificial Sequence
  Peptide
GMDYKDDDDKGYGRKKKRR

SEQ ID NO: 137 - protein, 7 aa, Artificial Sequence
  Peptide
RKRKRSR

SEQ ID NO: 138 - protein, 7 aa, Artificial Sequence
  Peptide
NYKKPKL

SEQ ID NO: 139 - protein, 8 aa, Artificial Sequence
  Peptide
KPKKKKEK

SEQ ID NO: 140 - protein, 13 aa, Artificial Sequence
  Peptide
RRHHCRSKAKRSR

SEQ ID NO: 141 - protein, 12 aa, Artificial Sequence
  Peptide
RRRRKRLSHRTA

SEQ ID NO: 142 - protein, 10 aa, Artificial Sequence
  Peptide
RRRVRRERNK

SEQ ID NO: 143 - protein, 7 aa, Artificial Sequence
  Peptide
RKRKRSR

SEQ ID NO: 144 - protein, 15 aa, Artificial Sequence
  Peptide
LFQRRRRGRGGRVTF

SEQ ID NO: 145 - protein, 18 aa, Artificial Sequence
  Peptide
GNYRRRRRRRGPKREGPR

SEQ ID NO: 146 - protein, 14 aa, Artificial Sequence
  Peptide
NQRRRRPLRRRDGT

SEQ ID NO: 147 - protein, 11 aa, Artificial Sequence
  Peptide
ARGRGRRRRLV

SEQ ID NO: 148 - protein, 13 aa, Artificial Sequence
  Peptide
AEARGRGRRRRLV

SEQ ID NO: 149 - protein, 11 aa, Artificial Sequence
  Peptide
IKRGRRRRRAP

SEQ ID NO: 150 - protein, 11 aa, Artificial Sequence
  Peptide
RRPRKRKRREK

SEQ ID NO: 151 - protein, 8 aa, Artificial Sequence
  Peptide
RKKRRRIK

SEQ ID NO: 152 - protein, 12 aa, Artificial Sequence
  Peptide
NRRPRKRKRREK

SEQ ID NO: 153 - protein, 9 aa, Artificial Sequence
  Peptide
FRRKRKRRA

SEQ ID NO: 154 - protein, 16 aa, Artificial Sequence
  Peptide
GKHRHERGHHRDRRER

SEQ ID NO: 155 - protein, 15 aa, Artificial Sequence
  Peptide
GRRRRATAKYRTSAH

SEQ ID NO: 156 - protein, 22 aa, Artificial Sequence
  Peptide
LRSARRRRRGRTDGRRFLLRRA

SEQ ID NO: 157 - protein, 12 aa, Artificial Sequence
  Peptide
SGARRRRVRCRK

SEQ ID NO: 158 - protein, 29 aa, Artificial Sequence
  Peptide
RPPGRKCGRCRRLANFPGRKRRRRRRKGL

SEQ ID NO: 159 - protein, 12 aa, Artificial Sequence
  Peptide
TRGARERCRRRR

SEQ ID NO: 160 - protein, 10 aa, Artificial Sequence
  Peptide
ERGDRRRRRC

SEQ ID NO: 161 - protein, 70 aa, Artificial Sequence
  Peptide
GPKRRSPEKRREGTRSADNTLERREKHEKRRDVSPERRRERSPTRRRDGSPSRRRRDRRRARSPERRRER

SEQ ID NO: 162 - protein, 12 aa, Artificial Sequence
  Peptide
YRRTVRLRRRLP

SEQ ID NO: 163 - protein, 9 aa, Artificial Sequence
  Peptide
EQRRRRRQV

SEQ ID NO: 164 - protein, 11 aa, Artificial Sequence
  Peptide
FLRRRRRFKKK

SEQ ID NO: 165 - protein, 8 aa, Artificial Sequence
  Peptide
FRRKRKRS

SEQ ID NO: 166 - protein, 10 aa, Artificial Sequence
  Peptide
SGRRRRRHHI

SEQ ID NO: 167 - protein, 16 aa, Artificial Sequence
  Peptide
SRRHHCRSKAKRSRHH

SEQ ID NO: 168 - protein, 19 aa, Artificial Sequence
  Peptide
GKKTGKNRKLKSKRVKPRD

SEQ ID NO: 169 - protein, 11 aa, Artificial Sequence
  Peptide
RKGKHKRKKLP

SEQ ID NO: 170 - protein, 18 aa, Artificial Sequence
  Peptide
GKRVAKRKLIEQNRERRR

SEQ ID NO: 171 - protein, 17 aa, Artificial Sequence
  Peptide
RKLKKNNNEKEDKRPRT

SEQ ID NO: 172 - protein, 18 aa, Artificial Sequence
  Peptide
GRKLKKKKNEKEDKRPRT

SEQ ID NO: 173 - protein, 18 aa, Artificial Sequence
  Peptide
GRRERNKMAAAKCRNRRR

SEQ ID NO: 174 - protein, 18 aa, Artificial Sequence
  Peptide
GRRERNKMAAAKCRNRRR

SEQ ID NO: 175 - protein, 19 aa, Artificial Sequence
  Peptide
GKRARNTEAAARRSRARKL

SEQ ID NO: 176 - protein, 9 aa, Artificial Sequence
  Peptide
RKKRRQRRR

SEQ ID NO: 177 - protein, 86 aa, Artificial Sequence
  Peptide
DPAGDLSAGFFMEELNTYRQKQGVVLKYQELPNSGPPHDRRFTFQVIIDGREFPEGEGRSKKEAKNAAAKLAVEILNKEKKAAALE

SEQ ID NO: 178 - protein, 87 aa, Artificial Sequence
  Peptide
GDPAGDLSAGFFMEELNTYRQKQGVVLKYQELPNSGPPHDRRFTFQVIIDGREFPEGEGRSKKEAKNAAAKLAVEILNKEKKCAALE

SEQ ID NO: 179 - protein, 87 aa, Artificial Sequence
  Peptide
GDPAGDLSAGFFMEELNTYRQKQGVVLKYQELPNSGPPHDRRFTFQVIIDGREFPEGEGRSKKEAKNAAAKLAVEILNKEKKACALE

SEQ ID NO: 180 - protein, 87 aa, Artificial Sequence
  Peptide
GDPAGDLSAGFFMEELNTYRQKQGVVLKYQELPNSGPPHDRRFTFQVIIDGREFPEGEGRSKKEAKNAAAKLAVEILNKEKKAACLE

SEQ ID NO: 181 - protein, 88 aa, Artificial Sequence
  Peptide
GDPAGDLSAGFFMEELNTYRQKQGVVLKYQELPNSGPPHDRRFTFQVIIDGREFPEGEGRSKKEAKNAAAKLAVEILNKEKKAAALEC

SEQ ID NO: 182 - protein, 87 aa, Artificial Sequence
  Peptide
GDPAGDLSAGFFMEELNTYRQKQGVVLKYQELPNSGPPHDRRFTFQVIICGREFPEGEGRSKKEAKNAAAKLAVEILNKEKKAAALE

SEQ ID NO: 183 - protein, 87 aa, Artificial Sequence
  Peptide
GDPAGDLSAGFFMEELNTYRQKQGVVLKYQELPNSGPPHDRRFTFCVIIDGREFPEGEGRSKKEAKNAAAKLAVEILNKEKKAAALE

SEQ ID NO: 184 - protein, 87 aa, Artificial Sequence
  Peptide
GDPAGDLSAGFFMEELNTYRQKQGVVLKYQELPNSGPPHDRRFTFQVCIDGREFPEGEGRSKKEAKNAAAKLAVEILNKEKKAAALE

SEQ ID NO: 185 - protein, 87 aa, Artificial Sequence
  Peptide
GDPAGDLSAGFFMEELNTYRQKQGVVLKYQELPNSGPPHDRRFTFQVIIDGCEFPEGEGRSKKEAKNAAAKLAVEILNKEKKAAALE

SEQ ID NO: 186 - protein, 87 aa, Artificial Sequence
  Peptide
GDPAGDLSAGFFMEELNTYRQKQGVVLKYQELPNSGPPHDRRFTFQVIIDGRECPEGEGRSKKEAKNAAAKLAVEILNKEKKAAALE

SEQ ID NO: 187 - protein, 86 aa, Artificial Sequence
  Peptide
GDPAGDLSAGFFMEELNTYRQKQGVVLKYQELPNSGPPHDRRFTFQVIIDGREFPGEGRSKKEAKNAAAKLAVEILNKEKKAAALE

SEQ ID NO: 188 - protein, 87 aa, Artificial Sequence
  Peptide
GDPAGDLSAGFFMEELNTYRQKQGVVLKYQELPNSGPPHDRRFTFQVIIDGREFPEGEGRSKKEAKNAAAKLAVEILCKEKKAAALE

SEQ ID NO: 189 - protein, 87 aa, Artificial Sequence
  Peptide
GDPAGDLSAGFFMEELNTYRQKQGVCLKYQELPNSGPPHDRRFTFQVIIDGREFPEGEGRSKKEAKNAAAKLAVEILNKEKKAAALE

SEQ ID NO: 190 - protein, 87 aa, Artificial Sequence
  Peptide
GDPAGDLSAGFFMEELNTYRQKQGVVLCYQELPNSGPPHDRRFTFQVIIDGREFPEGEGRSKKEAKNAAAKLAVEILNKEKKAAALE

SEQ ID NO: 191 - protein, 87 aa, Artificial Sequence
  Peptide
GDPAGDLSAGFFMEELNTYRQKQGVVLKYCELPNSGPPHDRRFTFQVIIDGREFPEGEGRSKKEAKNAAAKLAVEILNKEKKAAALE

(19) United States
(12) Patent Application Publication
(10) Pub. No.: US 2014/0335192 A1
(43) Pub. Date: Nov. 13, 2014

(54) DELIVERY OF RNA INTERFERING AGENTS

(71) Applicant: Kipperman, RM, Ch 7 BK Trustee for Traversa Therap, La Mesa, CA (US)

(72) Inventors: Gary H. WARD, San Diego, CA (US); Curt W. BRADSHAW, San Diego, CA (US)

(21) Appl. No.: 14/274,022

(22) Filed: May 9, 2014

Related U.S. Application Data
(60) Provisional application No. 61/821,649, filed on May 9, 2013.

(51) Int. Cl. C12N 15/113

(52) U.S. Cl. CPC C12N 15/113; C12N 2310/14; C12N 2310/3513
USPC 424/499; 530/395; 514/20.9

(57) ABSTRACT

Provided are compositions for the delivery of biomolecules, such as nucleic acids into target cells, and methods of making and using same. The compositions comprise nucleic acid delivery complexes that include a nucleic acid, such as an RNA interfering agent, an RNA neutralization domain, a double stranded RNA binding domain, and a protein transduction domain.

### RELATED APPLICATIONS

The present application claims priority to the U.S. Provisional Application Ser. No. 61/821,649, filed on May 9, 2013, the entire disclosure of which is incorporated herein by reference.

### BACKGROUND

1. Field of the Invention

The embodiments disclosed herein relate to methods and compositions useful for delivery of nucleic acids, including RNA interfering agents, into cells, tissues and organs.

2. Description of the Related Art

As the fields of gene therapy and molecular biology have developed rapidly, an urgent need has emerged to effectively deliver biomolecules, such as proteins and protein analogs, nucleic acids and nucleic acid analogs, including oligonucleotides such RNA, DNA hormones, small molecules, antiviral agents and the like into cells or tissues. Many therapeutic, research, and diagnostic applications rely upon the efficient transfer of biologically active molecules into cells, tissues, and organs.

The lipophilic and anionic nature of cell membranes, e.g., in mammalian cells, poses serious challenges for the delivery of negatively charged molecules, such as polyribonucleic acids, and polydeoxyribonucleic acids and analogs thereof, into the cells due to their size and charge. Various approaches to deliver negatively-charged biomolecules to cells and tissues have been tested, including viral-based delivery systems and non-viral based delivery systems.

Viral based gene nucleic acid delivery systems utilize retrovirus, adenovirus, and adeno-associated viruses. Virus-mediated nucleic acid delivery has drawbacks, however, including narrow range of cell infectivity, the elicitation of immune responses, and difficulty of large-scale production of viral vectors. (Yibin Wang et al., DDT. 5(1), 2000; Joanne T. Douglas. et al., Science & Medicine 44-52 (March/April), 1997). These shortcomings render viral based nucleic acid systems undesirable in therapeutic contexts.

Non-viral delivery systems include systems such as liposomes, polymers, calcium phosphate, electroporation, and micro-injection techniques (Saghir Akhtar et al., Adv. Drug Deliv. Rev. 44:3-21; Irina Lebedeva et al., Eur. J. Pharm. Biopharm. 50:101-119, 2000; Ch. Garcia-Chaumont et al., Pharmacol. Ther. 76:151-161, 2000). Ease of preparation and large-scale production have made the use of non-viral vectors a popular option for gene therapy. (Colin W. Pouton et al., Adv. Drug Deliv. Rev. 46:187-20, 2001).

Among the non-viral vectors developed to date, liposomes are the most frequently used gene transfer vehicle and are available commercially. Many liposomes are cationic. Cationic liposomes, complexed with nucleic acids or analogs thereof, electrostatically interact with the cell surface, and the complexes are then endocytosed into the cell cytoplasm. The cationic nature of the liposomes facilitates passage of negatively charged biomolecules such as polynucleotides across the cell membrane. However, while cationic liposomes mediate gene delivery effectively into cells in vitro, gene delivery in an in vivo system is quite limited as compared to viral vectors. Furthermore, the efficiency of gene delivery using cationic liposomes is generally dependent on the size of nucleic acids, and the cell line, even in an in vitro system. The major drawback of cationic liposomes, however, is their known cytotoxicity to cells (Saghir Akhtar et al., Adv. Drug Deliv. Rev. 44:3-21, 2000: Irina Lebedeva et al., Eur. J. Pharm. Biopharm. 50:101-119, 2000). The large, variable size and relative instability of liposomal complexes affects both the efficiency of nucleic acid delivery and the pharmacological viability of these delivery complexes. In addition to the adverse effects on nucleic acid delivery, the variability in liposomal complex size distributions adversely affects the quality control, scale-up, and long term shelf stability of liposomal complexes, rendering them problematic for pharmaceutical production.

Other cationic systems, such as cationic polymers, have been used to increase the efficiency of biomolecule delivery into cells. Polymers with numerous, positively-charged amine groups are able to bind strongly with nucleic acids, and also interact with the cell, so that the required amount of the polymers as compared to that of cationic liposomes can be reduced. However, cytotoxicity and insolubility of conventional cationic polymer approaches are drawbacks that limit the usefulness of cationic polymers alone as an effective gene delivery vehicle (Dan Luo et al., Nat. biotech. 18:33-37; Saghir Akhtar et al., Adv. Drug Deliv. Rev. 44:3-21, 2000).

Another non-viral system for the delivery of biomolecules relates to the addition of a covalently linked antibody to the oligonucleotide. The antibody-mediated approach to delivery of biomolecules is less than ideal, due to the therapeutic being shuttled down the endosomal pathway, leading to ultimate degradation of the biomolecule.

One approach to intracellular delivery of polynucleotides is set forth in Dowdy et al, US Published Application No. 2009/0093026. A complex of RNA with a protein transduction domain and a double-stranded RNA binding domain is used to facilitate intracellular delivery. This promising technology benefits from efficient in vitro polynucleotide delivery and low toxicity, as well as activity in a wide range of cell types. However, it has not been fully characterized in the art from a structural and mechanistic standpoint, nor optimized for performance or long term stability; rather, biological activity is demonstrated immediately after preparation of the complex, but decreases rapidly within hours, and is substantially reduced after 24 hours. Furthermore, the particle size of these complexes is very large (>300 nm), multimodal and highly variable, which limits its utility as a pharmaceutical preparation as well as for systemic delivery. Finally, the stoichiometry of these complexes is highly variable and undefined.

First, and importantly, the biomolecule delivery system should both protect the cargo nucleic acid molecule from degradation (physical, chemical and biological) during storage and/or transport. Second, the biomolecule delivery system should ensure the delivery and/or transport of the cargo nucleic acid molecule to and across the cellular membrane to the desired intracellular compartment or target. Third, the biomolecule delivery system should be homogeneous, structurally discrete and well-characterized, in order that it can be used in a pharmaceutical preparation. Fourth, the manufacturing process for the biomolecule delivery system should be reproducible and robust, generating a stable, efficacious product of the appropriate stoichiometry, size, and other important pharmaceutical properties. The biomolecule delivery systems described to date do not meet all of these criteria. Accordingly, there exists a need for improved biomolecule delivery systems that meet these criteria. The embodiments described herein address some or all of these needs.

### SUMMARY

The embodiments described herein relate to compositions and methods for delivering nucleic acids, such as RNA interfering agents, into target cells. The experiments and embodiments described herein are based, in part, upon the experiments described herein leading to the unexpected discoveries disclosed herein regarding the nature of particular biomolecule delivery agents. The discoveries disclosed herein have enabled the creation of a generation of nucleic acid delivery agents that are stable over time and provide efficient and predictable delivery of nucleic acids such as RNA interfering agents to target cells. Described herein are compositions of the new generation of nucleic acid delivery agents, as well as methods of making and using the same.

Accordingly, provided herein are methods of preparation and compositions for the intracellular delivery of nucleic acids, including RNA interfering agents. The compositions can advantageously include tight binding complexes that are substantially thermodynamically stable, and substantially in a low or lowest energy state. The complexes may further advantageously include moieties providing at least four functions: an RNA interfering agent, a double-stranded RNA binding domain, an RNA neutralization domain, and a protein transduction domain.

In a first aspect, provided herein are nucleic acid delivery complexes that include a double stranded RNA (dsRNA); an RNA neutralization domain (RND); a double-stranded RNA binding domain (DRBD) covalently attached to the RND to form an RND-DRBD chimera. The RND and DRBD of the RND-DRBD chimera can bind cooperatively and non-covalently to the dsRNA to form a strong, tightly bound dsRNA:RND-DRBD complex, wherein the complex is in a stable, low energy state. The nucleic acid delivery complexes also include a PTD covalently attached to the dsRNA:RND-DRBD complex, such that the PTD is free to interact with cell membrane to facilitate transport of the complex into a cell.

In some embodiments, the dsRNA is an siRNA, shRNA, miRNA, or the like.

In some embodiments, the RND-DRBD and dsRNA of the strong, tightly bound dsRNA:RND-DRBD complex have a Kₐ of 1×10⁻¹° or greater, e.g., a Kₐ of 1×10⁻⁹ or greater. For example, in some embodiments, the Kₐ of the RND-DRBD and dsRNA of the strong, tightly bound dsRNA:RND-DRBD complex is between about 1×10⁻¹° and 1×10⁻⁶. In some embodiments, the DRBD is bound to functional groups present in the major and minor grooves of the dsRNA through hydrogen bonds and nonpolar attractive forces, wherein the RND is a poly basic moiety which is bound to the dsRNA through strong ionic charge-charge interactions, and wherein the DRBD and RND together substantially neutralize the anionic and acidic functional groups of the dsRNA, and wherein the covalently bound PTD moiety is sufficiently free from strong ionic charge-based interaction with the dsRNA, and wherein the PTD of the nucleic acid delivery complex can interact with cell membranes, thereby rendering the nucleic acid delivery complex biologically active.

In some embodiments, wherein the nucleic acid delivery complex forms particles with a diameter of less than 300 nm, e.g., particles with a diameter of less than 200 nm.

In some embodiments, the DRBD and RND are covalently bound via a chemical crosslinker. In some embodiments, the RND-DRBD chimera is a recombinant protein. In some embodiments, the RND-DRBD recombinant protein can also include one or more PTDs, i.e., an RND-DRBD-PTD recombinant protein. In some embodiments, the RND-DRBD recombinant protein includes one or more PTDs that are covalently attached to the RND-DRBD recombinant protein via a chemical crosslinker. In some embodiments, one or more PTDs can be covalently linked to the RND domain. In some embodiments, one or more PTDs can be covalently linked to the DRBD domain.

Preferably, the dsRNA:RND-DRBD complex in the stable, low energy state has a ratio of dsRNA to RND-DRBD of about 1:0.1 to about 1:4, e.g., about 1:2.

In a second aspect, provided herein are methods for making a nucleic acid delivery complexes, as described above. The nucleic acid delivery complex can include a double stranded RNA (dsRNA), an RNA neutralization domain (RND), a double-stranded RNA binding domain (DRBD); and a protein transduction domain (PTD). The RND and DRBD can be covalently bound to each other to form an RND-DRBD chimera, and the RND-DRBD moieties of the RND-DRBD chimera bind cooperatively and non-covalently to the dsRNA to form a dsRNA:RND-DRBD complex having a stable, low energy state. The PTD (one or more) can be covalently attached to at least one reactive site present on the dsRNA:RND-DRBD complex such that the PTD is free to interact with cell membrane to facilitate transport of the complex into a cell. The method can include the steps of contacting the dsRNA with the RND-DRBD chimera, such that the RND and DRBD cooperatively bind the dsRNA to form a stable dsRNA:RND-DRBD complex. The DRBD moiety can be bound to the dsRNA through hydrogen bonds and nonpolar attractive forces, and the RND can be bound to the dsRNA through strong ionic charge-based interactions, such that the RND-DRBD chimera completely or substantially neutralizes the charge on the dsRNA. The method also includes the step of covalently attaching one or more PTDs to the DRBD, the dsRNA, or the RND, wherein at least one PTD is sufficiently free from charge-based interaction with the dsRNA that at least one PTD can facilitate transduction of the complex across a cell membrane.

In some embodiments, the contacting step is performed prior to the attaching step. In some embodiments, the attaching step is performed after the DRBD, dsRNA, and RND substantially achieve binding equilibrium. In some embodiments, the attaching step is performed prior to or contemporaneous with the contacting step.

In some embodiments, the RND-DRBD chimera is a recombinant protein. In some embodiments, the recombinant protein can further include at least one PTD. In some embodiments, at least one PTD can be covalently bound to the DRBD domain. In some embodiments, at least one PTD can be covalently bound to the RND domain. In some embodiments, at least one PTD can be covalently bound to the DRBD via a chemical crosslinker. In some embodiments, the at least one PTD can be covalently bound to the RND domain via a chemical crosslinker.

In some embodiments, the dsRNA:RND-DRBD complex in the stable, low energy state has a ratio of dsRNA to RND-DRBD of about 1:0.1 to about 1:4, e.g., about 1:2.

In some embodiments, the dsRNA is an siRNA. In some embodiments, the dsRNA is a miRNA. In some embodiments, the dsRNA is a shRNA.

In some embodiments, the RND-DRBD and dsRNA of the strong, tightly bound dsRNA:RND-DRBD complex have a Kₐ of at least 1×10⁻¹⁰, e.g., a Kₐ of 1×10⁻⁹ or greater. For example, in some embodiments, the Kₐ of the RND-DRBD and dsRNA of the strong, tightly bound RND-DRBD complex is between about 1×10⁻¹° and 1×10⁻⁶.

In a third aspect, provided herein is a double-stranded RNA (dsRNA) composition, that includes a complex of dsRNA, a double-stranded RNA binding domain (DRBD), and an RNA neutralization domain (RND), and at least one protein transduction domain (PTD) wherein the complex retains biological activity after formation for a period of at least 24 hours, wherein biological activity is defined as PTD-mediated intracellular delivery of biologically-active dsRNA.

In some embodiments, the complex forms particles with a diameter of less than 300 nm, e.g., less than 200 nm.

In some embodiments, the RND and the DRBD can be covalently bound to form an RND-DRBD chimera. In some embodiments, the RND-DRBD chimera is a recombinant protein. In some embodiments, the recombinant protein further includes at least one PTD. In some embodiments, at least one PTD can be covalently bound to the DRBD domain. In some embodiments, at least one PTD can be covalently bound to the RND domain. In some embodiments, at least one PTD can be covalently bound to the DRBD via a chemical crosslinker.

In some embodiments, the at least one PTD can be covalently bound to the RND domain via a chemical crosslinker. In some embodiments, the dsRNA:RND-DRBD complex in the stable, low energy state has a ratio of dsRNA to RND-DRBD of about 1:0.1 to about 1:4, e.g., about 1:2.

In some embodiments, the dsRNA is an siRNA. In some embodiments, the dsRNA is a miRNA. In some embodiments, the dsRNA is a shRNA.

In some embodiments, the RND-DRBD and dsRNA of the strong, tightly bound dsRNA:RND-DRBD complex have a Kₐ of at least 1×10⁻¹⁰, e.g., a Kₐ of 1×10⁻⁹ or greater. For example, in some embodiments, the Kₐ of the RND-DRBD and dsRNA of the strong, tightly bound RND-DRBD complex is between about 1×10⁻¹° and 1×10⁻⁶.

In a fourth aspect, provided herein are methods for making a pharmaceutical composition, which includes the step of forming a thermodynamically-stable complex of biologically-active double-stranded RNA (dsRNA), a double-stranded RNA binding domain (DRBD), an RNA neutralization domain (RND) and at least one protein transduction domain and the step of storing the complex for at least 24 hours while retaining activity (e.g., about 48 hours, 2 weeks, 1 month or more, wherein activity is defined as PTD-mediated intracellular delivery of biologically-active dsRNA.

In some embodiments, the RND and the DRBD can be covalently bound to form an RND-DRBD chimera. In some embodiments, the RND-DRBD chimera is a recombinant protein. In some embodiments, the recombinant protein further includes at least one PTD. In some embodiments, at least one PTD can be covalently bound to the DRBD domain. In some embodiments, at least one PTD can be covalently bound to the RND domain. In some embodiments, at least one PTD can be covalently bound to the DRBD via a chemical crosslinker.

In some embodiments, the dsRNA:RND-DRBD complex in the stable, low energy state has a ratio of dsRNA to RND-DRBD of about 1:0.1 to about 1:4, e.g., about 1:2.

In some embodiments, the dsRNA is an siRNA. In some embodiments, the dsRNA is a miRNA. In some embodiments, the dsRNA is a shRNA.

In some embodiments, the RND-DRBD and dsRNA of the strong, tightly bound dsRNA:RND-DRBD complex have a Kₐ of at least 1×10⁻¹⁰, e.g., a Kₐ of 1×10⁻⁹ or greater. For example, in some embodiments, the Kₐ of the RND-DRBD and dsRNA of the strong, tightly bound RND-DRBD complex is between about 1×10⁻¹° and 1×10⁻⁶.

In a fifth aspect, provided herein are methods for treating a subject with an RNA drug. The method can include providing a nucleic acid delivery complex described above, and administering the nucleic acid delivery complex to a subject such that the nucleic acid delivery complex undergoes PTD-mediated intracellular delivery of the dsRNA, after which the RNA drug exerts a desired pharmacological effect.

In a sixth aspect, provided herein are methods for treating a subject with an RNA drug. The method can include the step of providing a dsRNA composition described above, and administering the dsRNA composition to a subject such that dsRNA composition undergoes PTD-mediated intracellular delivery of the dsRNA, after which the RNA drug exerts a desired pharmacological effect.

In a seventh aspect, provided herein are pharmaceutical compositions that include the nucleic acid delivery complexes, or the dsRNA compositions described above, and one or more pharmaceutically acceptable excipients.

In some embodiments, the nucleic acid delivery complexes in the pharmaceutical composition form particles with a diameter of less than 300 nm, e.g., less than 200 nm.

In an eighth aspect, provided herein is a method of making a nucleic acid delivery complex. The method can include the steps of providing a RND-DRBD chimeric protein comprising an RNA neutralization domain (RND) and a double stranded RNA binding domain (DRBD), wherein the chimeric RND-DRBD protein includes a crosslink-reactive amino acid residue in either the RND or DRBD domain; providing a crosslinker-activated protein transduction domain (PTD), comprising a PTD and a chemical crosslinker, wherein the crosslinker is configured to react with the crosslink-reactive amino acid residue present in the chimera; complexing the RND-DRBD chimera with a double-stranded RNA (dsRNA) to form a thermodynamically stable dsRNA:RND-DRBD complex; contacting the crosslinker-activated PTD with the RND-DRBD chimera under conditions to form a covalent bond between the crosslink-reactive amino acid residue and the crosslinker of the crosslinker-activated PTD, to form an RND-DRBD-PTD structure.

In some embodiments, the contacting step is performed before the complexing step. In some embodiments, the complexing step is performed before the contacting step. In some embodiments wherein the complexing step is performed before the contacting step, the complexing step is performed at least 24 hours before the contacting step.

In some embodiments, the RND-DRBD-PTD structure comprises more than one PTD.

In some embodiments, the nucleic acid delivery complex has a ratio of dsRNA to RND-DRBD chimera of about 1:0.1 to about 1:4, e.g., about 1:2.

In some embodiments, the dsRNA is an siRNA. In some embodiments, the dsRNA is a microRNA. In some embodiments, the dsRNA is a shRNA.

In some embodiments, the RND-DRBD chimera and dsRNA of the thermodynamically stable complex have a Kₐ of at least 1×10⁻¹⁰, e.g., a Kₐ of 1×10⁻⁹ or greater. In some embodiments, the Kₐ of the RND-DRBD chimera and dsRNA of the thermodynamically stable complex is between about 1×10⁻¹° and 1×10⁻⁶.

In a ninth aspect, provided herein are methods of modulating expression of a target gene in a target cell. The methods can include the step of contacting the target cell with the nucleic acid delivery complex described above. In some embodiments, the method also includes the step of measuring the level of target gene expression in the target cell.

In some embodiments, the dsRNA is siRNA, and wherein said modulation is repression.

In a tenth aspect, provided herein are methods of modulating expression of a target gene in a target cell. The method can include the step of contacting the target cell with the dsRNA composition as described above. In some embodiments, the method also includes the step of measuring the level of target gene expression in the target cell.

In some embodiments, the dsRNA is siRNA, and wherein said modulation is repression.

### BRIEF DESCRIPTION OF THE DRAWINGS

FIG. 1 is a bar graph showing the % remaining expression of GADPH relative to control measured by RT-PCR, following treatment of human chondrocyte cells with dsGADPH siRNA:PTD-DRBD complexes as Described in Example 1. Reactions labeled “Cloudy Mixture” were not centrifuged prior to addition to the cells. Reactions labeled “Centrifuged Mixture” were centrifuged prior to addition to the cells. The experiments are described in Example 1.

FIG. 2 shows bar graphs depicting the % remaining expression of GADPH relative to control, measured by RT-PCR, following treatment of human chondrocyte cells with dsGADPH siRNA:DRBD-PTD complexes formed with different concentrations of GADPH siRNA relative to DRBD-PTD. dsGADPH siRNA:DRBD-PTD complexes were incubated for 30 min, 4 h, or 24 h, prior to addition to the cells. The experiments are described in Example 2.

FIG. 3 shows bar graphs depicting the % remaining expression of GADPH relative to control measured by RT-PCR, following treatment of human chondrocyte cells with dsGADPH siRNA:DRBD-PTD complexes formed using different ratios of GADPH siRNA relative to Tat-DRBD. dsGADPH siRNA:DRBD-PTD complexes were incubated for 30 min prior to addition to the cells. The experiments are described in Example 3.

FIG. 4 provides a table showing the size and peak area of particles of siRNA:DRBD-PTD complexes formed with the indicated ratios of DRBD-PTD to siRNA in PBS as measured by dynamic light scattering. The experiments are described in Example 3.

FIG. 5 provides a table showing the mean particle size of siRNA:DRBD-PTD complexes formed with different molar equivalents of DRBD-PTD to siRNA in water, pH 6.9 as measured by dynamic light scattering. The experiments are described in Example 3.

FIG. 6A-B are DSC thermograms of dsGADPH siRNA:DRBD-PTD complexes. The complexes were formed by mixing siRNA and DRBD-PTD chimera at a ratio of 1:2. DSC was performed immediately after mixing (FIG. 6A, T=0 hours) or 24 hours (FIG. 6B, T=24 hours).

FIG. 7 provides readouts from isothermal calorimetry experiments measuring the thermodynamics of association and stoichiometry of siRNA:DRBD binding in water (pH 6.0) and PBS (pH 7.0). The experiments are described in Example 3.

FIG. 8 provides readouts from isothermal calorimetry experiments measuring the thermodynamics of association and stoichiometry of siRNA:PTD binding in water (pH 6.0) and PBS (pH 7.0). The experiments are described in Example 3.

FIG. 9 provides readouts from isothermal calorimetry experiments measuring the thermodynamics of association and stoichiometry of siRNA:DRBD-PTD binding in water (pH 6.0) and PBS (pH 7.0). The experiments are described in Example 3.

FIGS. 10A-B are DSC thermograms of dsGADPH siRNA and dsGADPH siRNA:RND-DRBD complexes after the siRNA and RND-DRBD were incubated for 24 hours. The siRNA and DRBD-PTD chimera at a ratio of 1:2 in FIG. 10B.

FIG. 11 is a graph showing the % relative fluorescence of SYBR Gold added to siRNA:DRBD-PTD complexes formed with fixed concentrations of siRNA and different molar equivalents of DRBD-PTD and incubated for 30 min and for 24 h.

FIG. 12 is a table showing the zeta potential (expressed in mV) of various siRNA:RND-DRBD complexes formed using different molar ratios of siRNA:RND-DRBD. The experiments are described in Example 3.

FIG. 13 provide tables showing the zeta potential (expressed in (mV) of siRNA:RND-DRBD complexes formed by mixing siRNA and RND-DRBD chimera at a ratio of 1:2 at T=0 and T=24, in various solvents.

FIG. 14 shows the isothermal calorimetery data of a mixture of thermodynamically stable siRNA:RND-DRBD with PTD at the indicated molar ratios of siRNA:PTD. The experiments are described in Example 5.

FIG. 15 shows bar graphs depicting the % remaining expression of GADPH relative to control measured by RT-PCR, following treatment of human chondrocyte cells with dsGADPH siRNA:RND-DRBD complexes formed with different molar equivalents of activated PTD added to stable dsGADPH siRNA:RND-DRBD complexes. The bar graphs also show the % cell viability compared to control in the different reactions. The experiments are described in Example 6.

### DETAILED DESCRIPTION OF THE PREFERRED EMBODIMENT

Before the embodiments are further described, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the embodiments, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein may also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The embodiments disclosed herein are based, in part, upon Applicants' discovery of the unexpected composition of complexes that facilitate delivery of nucleic acids into cells. In particular, as described in further detail below, Applicants have made the unexpected discovery of various interactions and complexes formed between nucleic acids, such as RNA interfering agents, and other moieties including RNA neutralization domains, double stranded RNA binding domains, and protein transduction domains. In particular, Applicants have discovered that RNA neutralization domains and double-stranded RNA binding domains cooperate to bind nucleic acids such as RNA interfering agents to form stable, stoichiometric, thermodynamically-favored, low energy complexes of a size that are sufficiently small to enable cellular delivery (e.g., systemic or local injection) and which are stable over time. Applicants have further discovered that when the stable complexes further include a protein transduction domain that is free to interact with a cell membrane, the resulting complexes are of the size range that is appropriate for cellular administration and efficiently deliver the nucleic acid (e.g., RNA interfering agent) into cells.

Accordingly, provided herein are nucleic acid delivery complexes that include a nucleic acid, such as an RNA interfering agent, and groups providing the following functions: a double stranded RNA binding domain (“DRBD”), an RNA neutralization domain (“RND”), and a protein transduction domain. The DRBD and the RND cooperatively bind to the nucleic acid, e.g., RNA interfering agent, and can advantageously form a tightly-bound nucleic acid:RND-DRBD complex that exists in a low energy state. The nucleic acid delivery complex also includes a PTD attached to at least one moiety of the nucleic acid:RND-DRBD complex. The PTD is preferably sufficiently free from interactions with the rest of the complex that it can interact with a cell membrane to facilitate intracellular transport of the complex. For example, the PTD portion of the complex exhibits substantially no ionic, charge-based interaction, with the nucleic acid of the complex, e.g., RNA interfering agent. Polynucleotide delivery complexes described in the literature that include PTDs tend to complex the PTD with the nucleic acids over time to the extent that its intracellular transport properties are significantly attenuated or completely lost.

As used herein, the term “biomolecule” refers to any molecule of interest that is to be delivered to a target cell, including but not limited to, nucleic acids, proteins, glycoproteins, small molecules, and the like.

As used herein, the term “complex” refers to a multi-component entity, wherein the individual components of complex are either covalently attached, or attached or bound non-covalently (e.g., through ionic/electrostatic bonds, hydrogen bonds, Van der Waals forces, or the like). As described in further detail below, the nucleic acid delivery complexes and dsRNA complexes disclosed herein include moieties that provide at least four functions: i.e., a nucleic acid (e.g., an RNA interfering agent), a DRBD, a RND, and a PTD. In some embodiments, the DRBD(s), RND(s) and PTD(s) can be covalently linked, e.g., by virtue of being expressed as a recombinant chimeric protein, by virtue of being chemically synthesized as a chimeric protein, by virtue of being enzymatically joined, or by virtue of being chemically joined together via, e.g., crosslinkers or the like.

As discussed in further detail below, in some embodiments, the RND and DRBD together bind cooperatively and non-covalently to the nucleic acid, e.g, RNA interfering agent, to form a strong, tightly bound nucleic acid:RND-DRBD (e.g., dsRNA:RND-DRBD) complex. Within a relatively short time frame post-mixing, this complex exists in a stable, low energy state, as evidenced, for example, by reproducible and consistent stoichiometry, lack of change of charge state, function, morphological characteristics, and/or further binding interaction with the components of the complex. In some embodiments, a PTD is attached to at least one site on the DRBD, the RND, or the nucleic acid within the complex. In one embodiment, the attachment site or sites are predetermined, such that the PTD(s) is attached at only one site, two sites, three sites, or another specified number of sites. For example, in some embodiments, the PTD is not covalently linked to the nucleic acid of the nucleic acid delivery complex, but is covalently linked (either through a chemical crosslink or recombinant attachment) to either a RND and/or a DRBD that is non-covalently bound to the nucleic acid. In some embodiments, the PTD can be covalently linked, e.g., through a chemical crosslink, to the nucleic acid. In embodiments where the PTD is covalently linked to the nucleic acid, the PTD preferably does not substantially disrupt the non-covalent binding between the RND and/or DRBD with the nucleic acid.

In the context of the embodiments disclosed herein, the term “cooperative binding” and its variants describes a system in which at least three moieties are participating in a binding event, and the resulting binding between two of the moieties is facilitated by the presence of the third moiety. For example, a primary interaction between RND moieties and RNA is ionic or charge-based interaction with a relatively high Ka and relatively low enthalpy or AH. This event is kinetically favored and thus occurs relatively quickly, so that RNDs rapidly associate with RNA due to their difference in charge. On the other hand, DRBDs have been shown to bind in both the major and minor groove of dsRNA through hydrogen bonding and other non-ionic interactions with comparatively lower Kₐ and comparatively high enthalpy or AH. See, e.g., Nanduri et al. (1998) EMBO J. 17(18):5458-5465, see also Ryter et al. (1998) EMBO J. 17(24):7505-7513. Efficient, complete and stable binding occurs over time and may be dependent on alignment, conformation, and maintenance of the binding partners in proximity for a period of time sufficient to allow for counterion displacement from and complete binding of the DRBD and RND to the dsRNA in a manner which results in a thermodynamically stable state of the complex. When the DRBD is covalently bound to an RND, it is believed that the RND facilitates the binding of the DRBD to the dsRNA via its proximity and maintains that association to facilitate complete DRBD binding to the dsRNA. Without RND in the complex to maintain the DRBD in proximity of the dsRNA, the binding process between the DRBD and dsRNA is much less efficient. As an alternative or complementary definition, in the context of the embodiments described herein, two components of a complex are said to “bind cooperatively,” to a ligand wherein the affinity (Kₐ), or rate of binding of one or both components for the ligand changes, e.g., increases, in the presence of the other component or by an increase in the melting point (Tₘ) of the complex as compared to the Tₘ of the complex in the absence of the other component. By way of example, as discussed in further detail below, the DRBD(s) and RND(s) of the complexes disclosed herein “bind cooperatively” to nucleic acids (e.g., RNA interfering agents), as in the presence of an RND, (e.g., when the DRBD and RND are covalently linked), the Kₐ of the DRBD for nucleic acid is greater than the Kₐ of the DRBD for nucleic acids in the absence of RND. This binding is essentially stoichiometric, in that one dsRNA is bound to approximately two RND-DRBD molecules.

In the context of the embodiments disclosed herein, the term “stable” describes a state that, after reaching thermodynamic equilibrium (typically a time frame not exceeding 24 hours after mixing), does not substantially change afterwards over time. In contrast, the term “metastable” describes a system in a state that is not at thermodynamic equilibrium. By way of example, in the context of the compositions and methods disclosed herein, the term “stable” can refer to various characteristics of the compositions disclosed herein, including but not limited to, visual appearance (e.g., cloudy, clear, etc.), particle size, thermodynamic profile, or any other measurable characteristic. In some embodiments, the term “stable complex” refers to a composition that has been given sufficient time or presented with conditions which enable it to reach thermodynamic equilibrium and does not exhibit a significant change in physical state afterwards in at least a 4 hour, 6 hour, 8 hour, 10 hour, 12 hour, 14 hour, 16 hour, 18 hour, 20 hour, 22 hour, 24 hour, 26 hour 28 hour, 30 hour, 32 hour, 34 hour, 36 hour, 38 hour, 40 hour, 42 hour, 44 hour, 46 hour, 48 hour, 1 week, 2 week, 3 week, 4 week, 5 week, 6 week, 7 week, 8 week, 9 week, 10 week, or longer, period of time. By way of example, in some embodiments, a stable dsRNA:RND-DRBD complex can refer to a complex that exhibits little if any biological activity and no significant change in physical state over time. In some embodiments, a stable dsRNA:RND-DRBD complex can refer to a complex that does not change in size when in solution, over time (e.g., by forming colloidal particles or aggregates). In some embodiments, the stable complexes disclosed herein are thermodynamically stable. For example, a thermodynamically stable complex, as described herein, does not exhibit further complex formation binding heat upon the introduction of additional RND, DRBD, PTD, RND-DRBD, RND-DRBD-PTD, e.g., when analyzed using conventional techniques such as isothermal titration calorimetry or by the addition of intercalating agents which fluoresce upon binding to noncomplexed dsRNA.

The interactions between the different components of the nucleic acid delivery complexes are discussed in further detail below, in reference to the methods of making the same. Likewise, the arrangement of the different components of the nucleic acid delivery complexes is discussed in further detail below.

### Polynucleotides, Nucleic Acids, and RNA Interfering Agents

As used herein, the terms “nucleic acids,” “nucleotides” and “polynucleotides”, are generic to polydeoxyribonucleotides (containing 2-deoxy-D-ribose), to polyribonucleotides (containing D-ribose), to any other type of polynucleotide which is an N- or C-glycoside of a purine or pyrimidine base, and to other polymers containing non-nucleotidic backbones.

In some embodiments, the nucleic acids disclosed herein can include, for example, polyamide (e.g., peptide nucleic acids (PNAs) and polymorpholino, and other synthetic sequence-specific nucleic acid polymers provided that the polymers contain nucleobases in a configuration which allows for base pairing and base stacking, such as is found in DNA and RNA.

The terms nucleotide and polynucleotide include, for example, 3′-deoxy-2′,5′-DNA, oligodeoxyribonucleotide N3′→P5′ phosphoramidates, 2′-O-alkyl-substituted RNA, double- and single-stranded DNA, as well as double- and single-stranded RNA, DNA:RNA hybrids, and hybrids between PNAs and DNA or RNA.

The terms also include known types of modifications, for example, “caps,” (discussed in further detail below), labels which are known in the art, methylation, substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide.

In some embodiments, the nucleic acids disclosed herein include modifications to the phosphate backbone such as those described, for example, in International Patent Application Publication No., WO 2010/039543, International Patent Application Publication No. WO 08/008476, each of which is hereby expressly incorporated by reference in its entirety.

It will be appreciated that, as used herein, the terms “nucleoside” and “nucleotide” includes those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine. The resulting modified nucleotide or polynucleotide can form a base pair with other such modified nucleotidic units but not with A, T, C, G or U. For example, guanosine (2-amino-6-oxy-9-beta-D-ribofuranosyl-purine) may be modified to form isoguanosine (2-oxy-6-amino-9-beta-D-ribofuranosyl-purine). Such modification results in a nucleoside base which will no longer effectively form a standard base pair with cytosine. However, modification of cytosine (1-beta-D-ribofuranosyl-2-oxy-4-amino-pyrimidine) to form isocytosine (1-beta-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Mantsch et al. (1975) Biochem. 14:5593-5601, or by the method described U.S. Pat. No. 5,780,610 to Collins et al. The non-natural base pairs referred to as κ and π, can be synthesized by the method described in Piccirilli et al. (1990) Nature 343:33-37 for the synthesis of 2,6-diaminopyrimidine and its complement (1-methylpyrazolo[4,3]-pyrimidine-5,7-(4H,6H)-dione. Other such modified nucleotidic units which form unique base pairs have been described in Leach et al. (1992) J. Am. Chem. Soc. 114:3675-3683, or will be apparent to those of ordinary skill in the art.

The term “nucleic acid” or “polynucleotide” as used herein encompasses, among other things, single-stranded oligonucleotides, double-stranded oligonucleotides, double stranded RNA (dsRNA), double stranded DNA (dsDNA), RNA/DNA hybrids, and includes genomic DNA, cDNAs, chemically synthesized nucleic acids, isolated, naturally occurring nucleic acids, recombinant nucleic acids, and the like. The terms “nucleic acid” and “polynucleotide” includes, for example RNA interfering agents, such as dsRNA, siRNA, shRNA, antisense RNAs, microRNAs, and the like.

As used herein, the term “RNA interfering agent” refers to a class of polynucleotides that are capable of inhibiting or down-regulating gene expression, for example by mediating RNA interference or gene silencing in a sequence-specific manner. By way of example, RNA interfering agents can include, but are not limited to dsRNAs, including siRNAs, as well as shRNAs, miRNAs, and the like. By “inhibit,” “down-regulate” or “reduce” expression, it is meant that the expression of the gene product, and/or the level of the corresponding target mRNA molecules, and/or the level of activity of one or more gene products encoded by the target mRNA, is reduced below that observed in the absence of an RNA interfering agent, i.e., baseline or control levels. In some embodiments, the percent inhibition or down regulation is about 10%, 20%, 30%, 40%, 50%, 60%, 70%, 80%, 90%, or more. Accordingly, in some embodiments, the mRNA levels, gene product levels, or gene product activity of an “inhibited” or “reduced” or “down-regulated” target can be equal or greater than 10%, 20%, 30%, 40%, 50%, 60%, 70%, 80%, or 90%, of baseline levels, or activity.

As used herein, the term “dsRNA” is an abbreviation for “double-stranded RNA” and as used herein refers to a ribonucleic acid molecule having two substantially complementary RNA strands. The length of the dsRNAs can, for example, be anywhere from 10 nucleotides to about 100 nucleotides, or more, in length. In some embodiments, the dsRNAs can be siRNAs, shRNAs, pre-miRNAs, and the like. In some embodiments, the dsRNAs are cleaved, e.g., in vitro or in vivo, to siRNA. When “substantially complementary” used in the context of polynucleotides, refers to two strands of polynucleotides that are greater than 60%, preferably greater than 80%, and more preferably greater than 90%, e.g., greater than 95% complementary to each other, i.e., that the percent of bases that form hydrogen bonds to the cognate strand is about 60%, 70%, 80%, 90%, 91%, 92%, 93%, 94%, 95%, 96%, 97%, 98%, 99%, e.g., 100%.

As used herein, the term “siRNA” is an abbreviation for “short interfering RNA,” also sometimes known as “small interfering RNA” or “silencing RNA,” and refers to a class of double-stranded ribonucleic acid molecules that in eukaryotes are involved in the RNA interference (RNAi) pathway that mediates post-transcriptional, sequence-specific gene silencing. siRNAs are processed by the RNase III enzyme dicer. siRNAs hybridize to cognate mRNAs having sequences homologous to the siRNA sequence, and, as part of a large protein complex, and induce mRNA cleavage and degradation. In some embodiments, each strand of an siRNA can be about 15, 16, 17, 18, 19, 20, 21, 22, 23, 24, 25, 26, 27, 28, 29, or 30, or more, nucleotides in length. In some embodiments, the siRNAs can have a 3′ overhang of 1, 2, 3, or 4 nucleotides. In some embodiments, the siRNAs can have a 5′ overhang of 1, 2, 3, or 4 nucleotides. In some embodiments, the siRNAs can have both a 3′ and a 5′ overhang of 1, 2, 3, or 4 nucleotides. In some embodiments, the 3′ overhang is the same length as the 5′ overhang. In some embodiments, the 3′ overhang can be longer than the 5′ overhang by about 1, 2, 3, or 4 nucleotides. In some embodiments, the 3′ overhang can be shorter than the 5′ overhang, by about 1, 2, or 3 nucleotides. In some embodiments, the 5′ overhang can be longer than the 3′ overhang by about 1, 2, or 3 nucleotides. In some embodiments, the 5′ overhang can be shorter than the 3′ overhang by about 1, 2, or 3 nucleotides.

As used herein, the term “miRNA” is an abbreviation for “microRNA,” and refers to a class of dsRNA molecules that are processed by Dicer to form active single, stranded RNAs of about 21-23 nucleotides in length, which regulate gene expression. miRNA is complementary to a part of one or more messenger RNAs (mRNAs), and negatively regulate the expression of genes with sequences that are complementary to the miRNAs.

As used herein, the term “shRNA” is an abbreviation for “small hairpin RNA” or “short hairpin RNA.” shRNA is a sequence of ribonucleic acid that contains a sense sequence, antisense sequence, and a short loop sequence between the sense and antisense sequences. Due to the complementarity of the sense and antisense sequences, shRNA molecules tend to form hairpin-shaped double-stranded RNA (dsRNA). shRNA can be processed intracellularly by dicer into siRNA which then get incorporated into the siRNA induced silencing complex (RISC).

In some embodiments, the RNA interfering agents include a cap structure, as discussed above. By “cap structure” is meant chemical modifications, which have been incorporated at either terminus of the oligonucleotide (see, for example, Adamic et al., U.S. Pat. No. 5,998,203, incorporated by reference herein). The terminal modifications can function to protect the nucleic acid molecule from exonuclease degradation. The cap may be present at the 5′-terminus (5′-cap) or at the 3′-terminal (3′-cap) or may be present on both termini. Non-limiting examples of 5′-caps useful in the embodiments disclosed herein include, but are not limited to, a glyceryl, an inverted deoxy abasic residue (moiety); a 4′,5′-methylene nucleotide; a 1-(beta-D-erythrofuranosyl) nucleotide, a 4′-thio nucleotide; a carbocyclic nucleotide; a 1,5-anhydrohexitol nucleotide; L-nucleotides; alpha-nucleotides; a modified base nucleotide; a threo-pentofuranosyl nucleotide; an acyclic 3′,4′-seco nucleotide; an acyclic 3,4-dihydroxybutyl nucleotide; an acyclic 3,5-dihydroxypentyl nucleotide, a 3′-3′-inverted nucleotide moiety; a 3′-3′-inverted abasic moiety; 3′-2′-inverted nucleotide moiety; a 3′-2′-inverted abasic moiety; 1,4-butanediol phosphate; 3′-phosphoramidate; a hexylphosphate; an aminohexyl phosphate; a 3′-phosphate; a 3′-phosphorothioate; phosphorodithioate; or a bridging or non-bridging methylphosphonate moiety.

Non-limiting examples of 3′-caps useful in the embodiments disclosed herein include, but are not limited to, glyceryl; inverted deoxy abasic residues (moiety); 4′,5′-methylene nucleotides; a 1-(beta-D-erythrofuranosyl) nucleotide; a 4′-thio nucleotide, carbocyclic nucleotide; a 5′-amino-alkyl phosphate; a 1,3-diamino-2-propyl phosphate; a 3-aminopropyl phosphate; a 6-aminohexyl phosphate; a 1,2-aminododecyl phosphate; hydroxypropyl phosphate; a 1,5-anhydrohexitol nucleotide; an L-nucleotide; an alpha-nucleotide; a modified base nucleotide; a phosphorodithioate; a threo-pentofuranosyl nucleotide; an acyclic 3′,4′-seco nucleotide; a 3,4-dihydroxybutyl nucleotide; a 3,5-dihydroxypentyl nucleotide, a 5′-5′-inverted nucleotide moiety; a 5′-5′-inverted abasic moiety; a 5′-phosphoramidate; a 5′-phosphorothioate; a 1,4-butanediol phosphate; a 5′-amino; bridging and/or non-bridging 5′-phosphoramidate, a bridging or non bridging methylphosphonate and 5′-mercapto moieties (for more details see Beaucage and Lyer, 1993, Tetrahedron 49, 1925; incorporated by reference herein)

Non-limiting examples of RNA interfering agents useful in the embodiments disclosed herein include, but are not limited to those disclosed in U.S. Pat. Nos. 7,414,125; 7,414,109, 7,410,944; 7,405,292; 7,399,586; 7,304,042; 7,288,531; 7,235,654; 7,268,227; 7,173,015; 7,148,342; 7,199,109; 7,022,028; 6,974,680; 7,005,254; 7,307,067; 7,232,806; e.g, Let 7a, let 7a-1, let 7b, let 7b-1, let-7c, let-7d, let 7g, miR-1, miR-1-d, miR-1-2, miR-9, miR-10a, miR-10b, miR-15a, miR-16, miR-17, miR-17-3p, miR-18, miR-19a, miR-20, miR-21, miR-22, miR-23, miR-23a, miR-23b, miR-24, miR-25, miR-26a, miR-27a, miR-28, miR-29a, miR-29b, miR-30a-3p, miR-30a, miR-30e-5p, miR-31, miR-32, miR-34a, miR-92, miR-93, miR-95, miR-96, miR-98, miR-99a, miR-100, miR-101, miR-105, miR-106, miR-107, miR-108, miR-122, miR-124, miR-125, miR-125b, miR-126, miR-127, miR-128, miR-129, miR-130, miR-130a, miR-133, miR-133a, miR-133a-2, miR-133b, miR-134, miR-135, miR-137, miR-138, miR-139, miR-140, miR-141, miR-142, miR-143, miR-145, miR-147, miR-148, miR-149, miR-150, miR-152, miR-153, miR-154, miR-155, miR-181, miR-182, miR-183, miR-184, miR-186, miR-187, miR-188, miR-190, miR-191, miR-192, miR-193, miR-194, miR-195, miR-196, miR-197, miR-198, miR-199, miR-199a-1, miR-200b, miR-201, miR-203, miR-204, miR-206, miR-207, miR-208, miR-210, miR-211, miR-212, miR-213, miR-214, miR-215, miR-216, miR-217, miR-218, miR-222, miR-223, miR-224, miR-291-3p, miR-292, miR-292-3p, miR-293, miR-294, miR-295, miR-296, miR-297, miR-298, miR-299, miR-320, miR-321, miR-322, miR-324, miR-325, miR-326, miR-328, miR-329, miR-330, miR-331, miR-333, miR-335, miR-337, miR-338, miR-340, miR-341, miR-342, miR-344, miR-345, miR-346, miR-350, miR-367, miR-368, miR-369, miR-370, miR-371, miR-373, miR-380-3p, miR-409, miR-410, miR-412, or the like. Although exemplary antisense polynucleotides are described herein, the skilled artisan will readily appreciate that the compositions and methods disclosed herein are useful for any polynucleotides including RNA interfering agents such as siRNAs, miRNAs, shRNAs, dsRNAs, RNAi's, and oligonucleotides now known or discovered in the future. In a general sense, the operability of the methods and compounds disclosed herein is not dependent on the sequence or function of the oligonucleotide; rather, the disclosed methods and compounds are useful for delivering oligonucleotides (as a generic class) into cells. As such, the skilled artisan will appreciate that the polynucleotides (e.g. dsRNA, shRNA, miRNA, and the like) are not limited by any particular sequence. For example, in some embodiments, the sequence of the RNA interfering agent is at least 80%, e.g., 81%, 82%, 83%, 84%, 85%, 86%, 87%, 88%, 89%, 90%, 91%, 92%, 93%, 94%, 95%, 96%, 97%, 98%, 99%, or more complementary to a target sequence. For example, in some embodiments, the RNA interfering agent can include 1, 2, 3, 4, or 5 mismatches, with the target gene sequence. In some embodiments, the RNA interfering agent variants do not exhibit substantially different biological activity compared to the corresponding unmodified RNA interfering agent.

Any number of oligonucleotides or polynucleotides useful for diagnostics, therapeutics and research can be used in the methods and compositions disclosed herein.

### Polypeptides, Proteins

The nucleic acid delivery complexes disclosed herein include components that comprise or consist of polypeptides or proteins. As used herein, the term “polypeptide” or “protein” refers to a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

Variations in the sequence of the polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence of the polypeptide as compared with the native sequence polypeptide. Optionally the variation is by substitution of at least one amino acid with any other amino acid in one or more of the domains of the polypeptide. Guidance in determining amino acid residues that can be inserted, substituted or deleted without adversely affecting the desired activity may be found by comparing the sequence of the polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with an isoleucine, i.e., conservative amino acid replacements. Insertions or deletions may optionally be in the range of about 1 to 5 amino acids. The variation allowed can be determined by systematically making insertions, deletions or substitutions of amino acids in the sequence and testing the resulting variants for activity exhibited by the full-length or mature native sequence.

In particular embodiments, conservative substitutions of interest are shown below under the heading of preferred substitutions. If such substitutions result in a change in biological activity, then more substantial changes, denominated exemplary substitutions shown below, or as further described below in reference to amino acid classes, are introduced and the products screened.

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

The skilled artisan will appreciate that the term “peptide” or “polypeptide” encompasses sequences that include non-naturally occurring amino acids. Exemplary non-naturally amino acids include, but are not limited to, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

The skilled artisan will readily appreciate that the peptides and peptide domains described herein also include modified peptides such as glycoproteins, the L-optical isomer or the D-optical isomer of amino acids or a combination of both, as well as retro-inverso polypeptides. As used herein, the term “retro-inverso” refers a peptide that comprises an amino-carboxy inversion as well as enantiomeric change in one or more amino acids (i.e., levorotatory (L) to dextrorotary (D)). The peptides and domains described herein encompass D-amino acid modified polypeptides, amino-carboxy inversions of the amino acid sequence, amino-carboxy inversions containing one or more D-amino acids, naturally occurring proteins, recombinantly or synthetically synthesized peptides, non-inverted sequence containing one or more D-amino acids, peptidomimetics, Beta-amino acid analogs, gamma amino acid analogs, and the like.

The peptides disclosed herein encompass peptide fragments, e.g., functional fragments. As used herein, the term “functional fragment” refers to a portion of a polypeptide which exhibits at least one useful functional domain, such that the peptide fragment retains an activity of the polypeptide, e.g., RNA neutralizing activity, double-stranded RNA binding activity, transduction activity, and the like.

In some embodiments, the peptides disclosed herein can be chimeric proteins, or fusion proteins, or chimeric peptides, or fusion peptides, as discussed in further detail below. As used herein, the terms “chimeric protein”, “fusion protein”, “chimeric peptide” and “fusion peptide” are interchangeable, and refer to polypeptides which contain two or more functionally distinct regions, each made up of at least one functional monomer unit, e.g., a RNA neutralizing domain, a double stranded RNA binding domain, a protein transduction domain, or the like. As discussed in further detail below, chimeric polypeptides or fusion proteins described herein can be recombinant proteins encoded by nucleic acid comprising fusion of the coding sequence of one functional monomer to another functional monomer. In some embodiments, the chimeric or fusion proteins disclosed herein can be chemically synthesized as a single peptide using conventional polypeptide synthesis techniques. In some embodiments, the chimeric or fusion proteins disclosed herein can be created by using well-known chemical crosslinkers to join monomeric units together. In some embodiments, chimeric or fusion proteins can include a recombinant fusion joined via a chemical crosslinker to a separate monomeric unit.

The PTDs, DRBDs, and RNDs described are functionally-defined moieties. The term “moiety” is used herein in a broad context, to encompass discrete molecules as well as functional domains of a larger molecule, structure, or complex. These moieties can also be portions of fusion proteins, or can be linked together after synthesis or expression, through covalent or non-covalent linkages, or some or all of them can be linked to a carrier molecule or structure.

### RNA Neutralizing Domains

The nucleic acid delivery complexes described herein include, in addition to the nucleic acid (e.g., RNA interfering agent), one or more RNA neutralization domains (“RNDs”) that cooperatively bind with one or more DRBDs to the nucleic acid, to form a stable, low-energy nucleic acid:RND-DRBD complex. As used herein, the term “RNA neutralizing domain” refers to a domain that is polybasic or cationic in nature, that forms strong ionic charge-charge interactions with RNA (e.g., RNA interfering agents such as dsRNA, including siRNA) and that is capable of neutralizing the acidic and ionic functional groups of RNA, e.g., a RNA interfering agent. An RND may be comprised of one or more units, provided that they function together to neutralize the charge on the dsRNA. Each unit of the RND may be bound to a DRBD, dsRNA or another unit of the RND. The neutralizing effect of RNDs can be readily determined by, for example, measuring the charge state and/or zeta potential of nucleic acid:RND complexes, or any other method known to those skilled in the art. The zeta potential of a stable complex will be significantly less negative and/or positive as compared to uncomplexed dsRNA (highly negative), with values near neutrality, indicating that the majority of negative charge from the dsRNA have been neutralized by a positively charged binding partner functional group from the RND. Other measures of the neutralizing effect of the RND can be made and include assessment of charge state by imaged capillary Isoelectrophoresis (icIEF), a lack of additional binding upon addition of further quantities of RND and/or DRBD as measured by isothermal titration calorimetry, or by the absence of fluorescence when an intercalating dye is added (which normally fluoresces when free or unbound sites available on dsRNA are bound by the dye).

RNA neutralization domains can include, e.g., proteins, polymers, or other compounds that form ionic interactions with RNA, thereby neutralizing, or partially neutralizing the charge of the RNA.

The skilled artisan will appreciate that the number, length, amount of positive charge, and other properties of RNDs present in the embodiments disclosed herein can vary, provided that the negative charge of the nucleic acid (e.g., RNA interfering agent) within the nucleic acid:RND-DRBD complex is substantially or completely neutralized, bound and/or protected from substantial further interaction with other agents.

In some embodiments, neutralization of the nucleic acid by an RND(s), and/or DRBD(s), and/or RND-DRBD(s) results in protection of the nucleic acid from degradation by nucleases present in biological systems. For example, in some embodiments, RNA neutralization results in the protection of, e.g. a dsRNA, from RNAse digestion. As discussed further below, this can be measured in vitro or in vivo using routine methods.

In some embodiments, the overall zeta potential of the nucleic acid (e.g., RNA interfering agent), complexed with an RND or DRBD and/or RND-DRBD, is substantially, or near neutral. Zeta-potential, also called electrokinetic potential, is the electric potential at the surface of a particle relative to the potential in the bulk medium at a long distance. It can be measured according to conventional micro-electrophoresis analytical methods, e.g. via the determination of the velocity of the particles in a driving electric field by Laser-Doppler-Anemometry. By way of example, the Zetasizer Nano-ZS (Malvern Instruments) can be advantageously used to determine the zeta potential of particular embodiments. In some embodiments, the overall zeta potential of the nucleic acid (e.g., RNA interfering agent), complexed with RND-DRBD, and/or RND(s) and/or DRBD(s) in a stable, low energy nucleic acid:RND-DRBD complex, e.g., after binding equilibrium has been reached between the three components, is greater than about −10 mV, e.g., greater than about −9 mV, −8 mV, −7 mV, −6 mV, −5 mV, −4 mV, −3 mV, −2 mV, −1 mV, or more, or any fraction in between. Preferably, the zeta potential of the nucleic acid:RND-DRBD complex is substantially neutral. In this context, “substantially neutral” refers to a zeta potential between −10 mV and +10 mV. Most preferably, the zeta potential of the nucleic acid:RND-DRBD complex is between −5 mV and +5 mV, e.g., near neutral.

In some embodiments, RNA neutralization domains are characterized by their ability to protect RNA from nuclease digestion, e.g., by RNAse.

In some embodiments, RNA neutralization domains are characterized by their ability to electrostatically bind to an RNA, such as an RNA interfering agent, quickly when in solution, but with relatively low enthalpy, e.g., when compared to a double-stranded RNA binding domain (“DRBD”). By way of example, in some embodiments, the RNA neutralization domain can bind to a nucleic acid, such as an RNA interfering agent, with a Kₐ of about 1×10⁷ or greater, e.g., 1×10⁸, 1×10⁹, or greater, at a stoichiometry ranging from about 1:1 to 1:10 of dsRNA:RND, depending on the size of the dsRNA.

In some embodiments, RNA neutralization domains are characterized by their ability to cooperatively bind to dsRNAs, such as RNA interfering agents, with DRBDs. Further, the RNA neutralization domains in the complexes disclosed herein are characterized in their direct interaction, e.g., through non-covalent binding, with the nucleic acids of the nucleic acid delivery complex. In some embodiments, the RNDs are characterized by their formation of strong, ionic interactions with the phosphate and other negatively charged groups present in nucleic acids. RNDs of the nucleic acid delivery complexes disclosed herein are further characterized in that, once present in a stable, low-energy nucleic acid:RND-DRBD complex, as described herein, the RNDs do not substantially facilitate delivery of the nucleic acid across cell membranes. Delivery of nucleic acids can be measured using any technique known to those skilled in the art, including, but not limited to, measuring the delivery of fluorescently labeled nucleic acids into cells, and/or measurement of the level of activity of biologically active nucleic acids. By way of example, the measurement of relative RNA interference by RNA interfering agents can be assessed, as described elsewhere, by calculating the relative % of target mRNA present in cells following delivery, calculating the relative % of target gene product activity of cells following delivery, and the like. For example, in some embodiments, RND-containing stable, low-energy RNA interfering agent:RND-DRBD complexes provide a less than 20%, less than 15%, less than 10% less than 5%, or less, inhibition of target mRNA, gene product, or gene product activity when used to treat target cells, compared to control or mock-treated cells.

The skilled artisan will appreciate that in some embodiments, an RND and/or units of an RND can include sequences that are similar, or the same as certain PTD domains. However, it will be appreciated that the RNDs and PTDs, as described herein are primarily functionally, but also structurally defined. As such, the RNDs of the embodiments disclosed herein lack PTD functionality, e.g., do not efficiently deliver the nucleic acids in which they are in a stable complex, across cell membranes and are thus not PTDs. Likewise, the PTDs disclosed herein do not form substantial ionic interactions with the nucleic acids of the stable complexes disclosed herein, and do not function as, and are thus not considered, RNDs.

The skilled artisan will appreciate that there is a relationship between the amount of positive charge and/or length and/or other properties of a given RND and the amount of negative charge and/or length of dsRNA such that substantial or complete dsRNA neutralization is achieved. Given the structural and charge variation that exists within molecules of the nucleic acid class, the properties of an RND are preferably appropriately matched to that of the dsRNA in which it is intended to complex in order that complete or nearly complete dsRNA neutralization is achieved.

In some embodiments, the RND can be a polypeptide, e.g., that has an overall cationic charge, or can be a non-peptide cationic moiety. In some embodiments, the RNDs can comprise several subunits, wherein together the subunits form the RND that is capable, when associated with one or more DRBDs, of substantially neutralizing the charge of the RNA interfering agent, e.g., dsRNA such as siRNA. Exemplary peptide RNDs and RND subunits useful in the embodiments disclosed herein include, but are not limited to, peptides comprising or consisting of poly-arginine sequences, i.e., between about 4 and 20 consecutive arginine residues, peptides comprising or consisting of poly-lysine sequences, i.e., between about 4 and 20 consecutive lysine residues, peptides comprising or consisting of poly-histidine residues, i.e. between about 4 and 20 consecutive histidine residues, polypeptides that contain only arginine and lysine residues, polypeptides that contain only arginine and histidine residues, polypeptides that contain only histidine and lysine residues, peptides that comprise or consist of spaced arginine residues as described, for example, in U.S. Pat. No. 7,585,834, or modifications thereof, peptides that comprise, or consist of spaced lysine residues, peptides that comprise or consist of spaced histidine residues, and the like.

In some embodiments, the RNDs or RND subunits can include a biostere. For example, in some embodiments, one or more arginine residues of the RND can be substituted with a modified lysine wherein the guanidine is replaced with a bioisotere, e.g., those described in International Patent Application Publication No. WO 05/123700.

In some embodiments, the RND or RND subunit can comprise polypeptides, including protamine, HIV TAT, or functional fragments thereof. In some embodiments, the RND can comprise a polypeptide sequence comprising a functional fragment of the Antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the cationic N-terminal domain of prion proteins, and/or and Buforin II (Park et al. (2000) Proc. Natl. Acad. Sci. U.S.A. 97:8245-50), or the like, or fragments thereof.

In some embodiments, the RNDs comprise polybasic sequences with one or more amino acid substitutions. By way of example, in some embodiments, the RND or RND subunit can consist of or comprise the following sequences:

(SEQ ID NO: 1)
MGRKKRRQRRRGHSGRKKRRQRRRGHIYPYDVPDYAGDPGRKKRRQRR
RG;
(SEQ ID NO: 2)
GYAAARRRRRRRGHSGYAAARRRRRRRGHIYPYDVPDYAGDPGYAAAR
RRRRRR;
(SEQ ID NO: 3)
GYAAARRRRRRRGHSGYAAARRRRRRRGHIYPYDVPDYAGDPGYAAAR
RRRRRR
(SEQ ID NO: 4)
GYAAARRRRRRRGHSGYAAARRRRRRRGSGSGYAAARRRRRRR
(SEQ ID NO: 5)
GYARRRRRRRRRGHSGYARRRRRRRRRGHIYPYDVPDYAGDPGY
ARRRRRRRRR
(SEQ ID NO: 6)
GYARRRRRRRRRGHSGYARRRRRRRRRGSGSGYARRRRRRRRR;
or
(SEQ ID NO: 7)
RRRRRRR

Yet additional RNDs useful in the embodiments disclosed herein include a fragment of HIV TAT in which the TAT 49-56 sequence has been modified so that at least two basic amino acids in the sequence are substantially aligned along at least one face of the TAT fragment. Exemplary TAT fragments useful as RNDs in the embodiments disclosed herein can include at least one specified amino acid substitution in at least amino acids 49-56 of TAT which substitution aligns the basic amino acid residues of the 49-56 sequence along at least one face of the segment.

In some embodiments, the RND domain comprises a peptide represented by the following general formula: B1-X1-X2-X3-B2-X4-X5-B3, wherein B1, B2, and B3 are each independently a basic amino acid, such as lysine or arginine, the same or different; and X1, X2, X3, X4 and X5 are each independently an alpha-helix enhancing amino acid, such as alanine, the same or different.

In some embodiments, the RND domain comprises a polypeptide represented by the following general formula: X-X-R-X-(P/X)-(B/X)-B-(P/X)-X-B-(B/X), wherein X is any alpha helical promoting residue such as alanine; P/X is either proline or X as previously defined; B is a basic amino acid residue, e.g., arginine (Arg) or lysine (Lys); R is arginine (Arg) and B/X is either B or X as defined above.

In some embodiments, the RND can include between 7 and 10 amino acids and have the general formula K-X1-R-X2-X1 wherein X1 is R or K and X2 is any amino acid.

Yet other exemplary RND sequences can be derived from the sequences described in International Patent Application Publication No. WO 08/008476 and WO 07/095152, WO 03/059940, WO 03/059941, WO 09/041902, WO 05/084158; WO 00/062067, WO 00/034308, and WO 99/55899, U.S. Patent Application Publication No's 2002/0102265, 2003/0082561, 2002/0102265, 2003/0040038, the disclosures of which relating to polypeptide sequences are hereby expressly incorporated by reference.

By way of example, some amino acid sequences useful as units of RNDs and/or RNDs in the embodiments disclosed herein include, but are not limited to those provided in Table 1 below:

TABLE 1

| | SEQ ID |
|---|---|
| SEQUENCE | NO: |
| YARRKKARRQARR | 8 |
| YGRKKRRQRRR | 9 |
| AKIWFQNRRMKWKKENN | 10 |
| DAATATRGRSAASRPTERPRAPARSASRPRRPVE | 11 |
| RQIKIWFQNRRMKWKK | 12 |
| TRSSRAGLQFPVGRVHRLLRK | 13 |
| GWTLNSAGYLLGKINKALAALAKKIL | 14 |
| KLALKLALKALKAALKLA | 15 |
| MANLGYWLLALFVTMWTDVGLCKKRPKP | 16 |
| LLIILRRRIRKQAHAHSK | 17 |
| KETWWETWWTEWSQPKKKRKV | 18 |
| RGGRLSYSRRRFSTSTGR | 19 |
| SDLWEMMMVSLACQY | 20 |
| TSPLNIHNGQKL | 21 |
| KRRQRRR | 22 |
| RKKRRQR | 23 |
| RKKRRQRR | 24 |
| GRKKRRQRRRPPQ | 25 |
| TRQARRNRRRRWRERQR | 26 |
| TRRQRTRRARRNR | 27 |
| TRRNKRNRIQEQLNRK | 28 |
| TAKTRYKAEEAELIAERR | 29 |
| MDAQTRRRERRAEKQAQWKAAN | 30 |
| RRRRNRTRRNRRRVR | 31 |
| KMTRAQRRAAARRNRWTAR | 32 |
| TRRQRTRRARRNR | 33 |
| TRQARRNRRRRWRERQR | 34 |
| GRKKRRQRRRPPQ | 35 |
| RRRQRRKKR | 36 |
| AGRKKRRQRRR | 37 |
| YARKARRQARR | 38 |
| YARAAARQARA | 39 |
| YARAARRAARR | 40 |
| YARAARRAARA | 41 |
| YARRRRRRRRR | 42 |
| YAAARRRRRRR | 43 |
| KKRPKPG | 44 |
| KRPAATKKAGQAKKL | 45 |
| PKKKRKV | 46 |
| GGG(ARKKAAKA)₄ | 47 |
| KKKKKKKGGC | 48 |
| RQIKIWFQNRRMKWKK | 49 |
| GWTLNSAGYLLGKINLKALAALAKISIL | 50 |
| AGYLLGKINLKALAALAKKIL | 51 |
| DAATATRGRSAASRPTERPRAPARSASRPRRPVD | 52 |
| GCRKKRRQRRRPPQC | 53 |
| TRQARRNRRRRWRERQR | 54 |
| RRRRNRTRRNRRRVR | 55 |
| (R)ₙ, Sterylated (R)₈(R)ₙGC (n = 7-16) | 56 |
| GLFEAIAGFIENGWEGMIDG | 57 |
| GLFKAIAKFIKGGAWKGLIKG | 58 |
| GLFKAIAEFIEGGWEGLIEG | 59 |
| GLFKAIAEFIEGGWEGLIEGCA | 60 |
| GLFKAIAEFIEGGWEGLIEGWYG | 61 |
| GLFHAIAHFIEGGWHGLIHGWYG | 62 |
| WEAALAEALAEALAEALAEHLAEALAEALEALAA | 63 |
| WEAKLAKALAKALAKHLAKALAKALKACEA | 64 |
| KKALLALALHHLAHLALHLALALKKA | 65 |
| KKALLAHALHLLALLALHLAHALKKA | 66 |
| KKALLALHALHHLALLAHHLAHALKKA | 67 |
| KKHLLAHALHLLALLALHLAHALAHLKKA | 68 |
| GLFEALLELLESLWELLLEA | 69 |
| GLFKALLKLLKSLTKLLLKA | 70 |
| GLFRALLRLLRSLTALLLRA | 71 |
| PKKKRKVEDPYC | 72 |
| SSDDEATADSQHSTPPKKKRKVEDPYC | 73 |
| MRRAHHRRRRASHRRMRGG | 74 |
| GALFLGFLGAAGSTMGAWSQPKSKRKV | 75 |
| MPKTRRRPRRSQRKRPPTWAHFPGFGQGSLC | 76 |
| GIGAVLKVLTTGLPALISWIKRKRQQ | 77 |
| CLIKKALAALAKLNIKLLYGASNLTWG | 78 |
| GALFLGFLGAAGSTMGAWSQPKKKRKV | 79 |
| GALFLAFLAAALSLMGLWSQPKKKRKV | 80 |
| GLFGAIAGFIENGWEGMIDGRQIKIWFQNRRMKWKK | 81 |
| MVKSKIGSWILVLFVAMWSDVGLCKKRPKP | 82 |
| LIRLWSHLIHIWFQNRRLKWKKK | 83 |
| LLGDFFRKSKEKIGKEFKRIVQRIKDFLRNLVPRTES | 84 |
| SWLSKTAKKLENSAKKRISEGIAIAIQGGPR | 85 |
| ACYCRIPACIAGERRYGTCIYQGRLWAFCC | 86 |
| FITKALGISYGRKKRRQRRRPPQC | 87 |
| LGISYGRKKRRQRRRPPQC | 88 |
| KKWKMRRNQFWIKIQR | 89 |
| RQIKIWFQNRRMKWKK | 90 |
| RQIKIWFPNRRMKWKK | 91 |
| RQPKIWFPNRRMPWKK | 92 |
| RQIKIWFQNMRRKWKK | 93 |
| RQIRIWFQNRRMRWRR | 94 |
| RRWRRWWRRWWRRWRR | 95 |
| IKIWFQNRRMKWKK | 96 |
| RRMKWKK | 97 |
| WFQNRRMKWKK | 98 |
| IWFQNRRMKWKK | 99 |
| KIWFQNRRMKWKK | 100 |
| RQIKIWFQNRRMKWK | 101 |
| RQIKIWFQNRRM | 102 |
| RQIKIFFQNRRMKFKK | 103 |
| TERQIKIWFQNRRMK | 104 |
| KIWFQNRRMK | 105 |
| GWTLNSAGYLLGPINKALAALAKKIL | 106 |
| KLALKLALKAWKAALKLA | 107 |
| KLALKAALKAWKAAAKLA | 108 |
| KLALKAAAKAWKAAAKAA | 109 |
| KITLKLAIKAWKLALKAA | 110 |
| KIAAKSIAKIWKSILKIA | 111 |
| KALAKALAKLWKALAKAA | 112 |
| KLALKLALKWAKLALKAA | 113 |
| KLLAKAAKKWLLLALKAA | 114 |
| KLLAKAALKWLLKALKAA | 115 |
| KALKKLLAKWLAAAKALL | 116 |
| KLAAALLKKWKKLAAALL | 117 |
| KALAALLKKWAKLLAALK | 118 |
| ALALQLALQALQAALQLA | 119 |
| ELALELALEALEAALELA | 120 |
| NKIPIKD | 121 |
| CGPGSDDEAAADAQHAAPPKKKRKVGY | 122 |
| KRPAATKKAGQAKKKKL | 123 |
| RRNRRRRW | 124 |
| APKRKSGVSK | 125 |
| VQRKRQKLMP | 126 |
| DTWTGVEALIRILQQLLFIHFRIGCRHSRIGIIQQRRTRN | 127 |
| GA | |
| MGLGLHLLVLAAALQGAWSQPKKKRK | 128 |
| AAVALLPAVLLALLAPVQRKRQKLMP | 129 |
| NAKTRRHERRRKLAIER | 130 |
| KLTRAQRRAAARKNKRNTR | 131 |
| TAKTRYKARRAELIAERR | 132 |
| RVIRVWFQNKRCKDKK | 133 |
| LLGKINLKALAALAKKIL | 134 |
| GIGAVLKVLTTGLPALISWIKRKRQQ | 135 |
| GMDYKDDDDKGYGRKKKRR | 136 |
| RKRKRSR | 137 |
| NYKKPKL | 138 |
| KPKKKKEK | 139 |
| RRHHCRSKAKRSR | 140 |

Table 2, below lists various peptide sequences derived from human proteins that are useful RNDs or RND subunits in the embodiments disclosed herein. Table 2 lists either the name of the parent protein or accession number and the relevant sequence.

TABLE 2

| | | SEQ |
|---|---|---|
| | | ID |
| Derived From: | SEQUENCE | NO: |
| HNF3 | RRRRKRLSHRT(A) | 141 |
| cAmp dep TF/fos | RRRVRRERNK | 142 |
| related antigen | | |
| Histone H2B | RKRKRSR | 143 |
| NP 001182188.1 | LFQRRRRGRGGRVTF | 144 |
| NP 001182056.1 | GNYRRRRRRRGPKREGPR | 145 |
| NP 001180317.1 | NQRRRRPLRRRDGT | 146 |
| ADK 45392.1 | ARGRGRRRRLV | 147 |
| NP 001170847.1 | AEARGRGRRRRLV | 148 |
| Q5VYJ5.3 | IKRGRRRRRAP | 149 |
| NP001180259.1 | RRPRKRKRREK | 150 |
| BAH14741.1 | RKKRRRIK | 151 |
| BAG6220.1 | NRRPRKRKRREK | 152 |
| NP997309.2 | FRRKRKRRA | 153 |
| Cyclin | GKHRHERGHHRDRRER | 154 |
| HEN1/NSCL1 | GRRRRATAKYRT(S)AH | 155 |
| NP 001106970.2 | LRSARRRRRGRTDGRRFLLRRA | 156 |
| BAJ 05817.1 | SGARRRRVRCRK | 157 |
| NP 001159818.3 | RPPGRKCGRCRRLANFPGRKRRRRRRKGL | 158 |
| NP 001164058.1 | TRGARERCRRRR | 159 |
| NP 001164100.1 | ERGDRRRRRC | 160 |
| Q96QZ7.3 | GPKRRSPEKRREGTRSADNTLERREKHEK | 161 |
| | RRDVSPERRRERSPTRRRDGSPSRRRRDR | |
| | RRARSPERRRER | |
| NP001013649.2 | YRRTVRLRRRLP | 162 |
| NP001156793.1 | EQRRRRRQV | 163 |
| ACS83750.1 | FLRRRRRFKKK | 164 |
| ADN03320.1 | FRRKRKRS | 165 |
| BAG65579 | SGRRRRRHHI | 166 |
| hPER1 | SRRHHC [symbol] SKAKRSRHH | 167 |
| hPER2 | GKKTGKNRKLKSKRVKPRD | 168 |
| hPER3 | RKGKHKRKKLP | 169 |
| hThyroid alpha 1 | GKRVAKRKLIEQNRERRR | 170 |
| HMK1 | RKLKKNNNEKEDKRPRT | 171 |
| HME1 | GRKLKKKKNEKEDKRPRT | 172 |
| C-Fos | GRRERNKMAAAKCRNRRR | 173 |
| Nucleoplasmin | GRRERNKMAAAKCRNRRR | 174 |
| GCN-4 | GKRARNTEAAARRSRARKL | 175 |

Other RNDs useful in the embodiments described herein can be derived from penetratin (1OMQ_A), pVECP101 (ACT78456), MATa2 (Q6B184), HIV-1 rev (CAA41586), NF-kappaB (NP_{—}003989), M9 (BAA76626), Vpr (BAH97661), FP_NLS (MPG), Sp-NPS (ACU27162), SN50, Importins and Karyopherins, e.g., Karyopherin alpha (NP 002255), and Karyopherin beta (NP 002256), and the like.

In some embodiments, the RND or RND subunit can be a non-protein or non-peptide moiety. For example, in some embodiments, the RND can be a cationic polymer, or cationic polymer conjugate. Non-limiting examples of cationic polymers, or cationic polymer conjugates useful in the embodiments disclosed herein include, but are not limited to cationic polymers such as polyetheleneimine (PEI), cationic polymer conjugates obtained by conjugating polyethyleneimine and hyaluronic acid (e.g., as described in U.S. Patent Application Publication No. 2010/00144035), PEI-containing polymers such as those disclosed in U.S. Patent Application Publication No. 2010/00210715), deacylated PEI, N1, N12-bis(ethyl) spermine (see, e.g., Bartzatt, R. et al. 1989. Biotechnol. Appl. Biochem. 11:133; Wagner E. et al. 1992. Proc. Natl. Acad. Sci. 88:4255), polyvinylpyrrolidone (PVP), chitosan, polyphosphates, polyphosphoesters (see U.S. Pub. No. 2002/0045263), poly(N-isopropylacrylamide), etc. Certain of these polymers comprise primary amine groups, imine groups, guanidine groups, and/or imidazole groups. Some examples include poly(β-amino ester) (PAE) polymers (such as those described in U.S. application Ser. Nos. 09/969,431 and 10/446,444; and U.S. Patent Application Publication No. 2002/0131951). In some embodiments, the cationic polymers are linear. In some embodiments, the cationic polymers comprise tertiary or quaternary amines. In some embodiments, the cationic polymers are branched. In some embodiments, the RND comprises blends, copolymers, and modified cationic polymers.

In some embodiments, the RND or RND subunit can comprise a dendrimer. As used herein, the term “dendrimer” refers to polymers typically ranging from 1 to about 20 nanometers in diameter having a tree-like, branching morphology. Exemplary dendrimers useful in the embodiments disclosed herein can include, but are not limited to, polyamidoamine (PAMAM), polypropylamine (POPAM), polyethylenimine, iptycene, aliphatic poly(ether), and/or aromatic polyether dendrimers (see, e.g., U.S. Pat. No. 6,471,968).

In some embodiments, the RND can include both protein, and non-protein components, such as a polymer that has been modified with amino acid residues, e.g., lysine residues, arginine residues, histidine residues, or the like, including but not limited to those described in U.S. Patent Application Publication No. 2010/02024301.

In some embodiments, the compositions disclosed herein include 1, 2, 3, 4, 5, 6, 7, 8, 9, 10, or more RND subunits. In some embodiments, the RND can include two or more different subunits, or two or more of the same subunits. In some embodiments, the RND is not contiguous, i.e., the subunits of the RND are separated by one or more spacers, domains, e.g., DRBDs, targeting domains, or the like, provided that the RND subunits within the chimera together substantially neutralize the RNA interfering agent when covalently bound to one or more DRBDs.

The RNDs of the embodiments disclosed herein can include any of the full length polypeptides described herein, as well as fragments or variants thereof, including as modified polypeptides, e.g., including peptide variants that have amino acid substitution(s), e.g., between about 1, 2, 3, 4, 5, 6, 7, 8, 9, 10, 11, 12, 13, 14, 15, 16, 17, 18, 19, 20, 21, 22, 23, 24, 25, or more, amino acid substitutions. In some embodiments, the modified variants of the RNDs described herein do not exhibit substantially less biological activity, e.g., affinity for nucleic acids, association with nucleic acids, binding to nucleic acids, neutralization of nucleic acids, or other, compared to the corresponding unmodified RND.

### Protein Transduction Domains

The nucleic acid delivery complexes disclosed herein can include a protein transduction domain (“PTD”), or “cell penetrating peptide (CPP).” As used herein, the terms “protein transduction domain” and “cell penetrating peptide” are interchangeable, and refer to a polypeptide that, when present and active in a nucleic acid delivery complex disclosed herein, facilitates the delivery and cytoplasmic transport of nucleic acids, e.g., RNA interfering agents, into a target cell. In the context of the compositions disclosed herein, a PTD can refer to an entity that will manifest cell entry and exit rates (referred to as k₁ and k₂, respectively), that favor transport of at least picomolar amounts of the nucleic acid (e.g., RNA interfering agent) of the complex, into a target cell. The skilled artisan will appreciate that several conventional techniques can be employed to perform the kinetic analysis and calculate k₁ and k₂ values. Preferably, a PTD imparts upon the nucleic acid delivery complexes and the dsRNA complexes disclosed herein, an entry to exit rate ratio that provides a gene silencing effect. In some embodiments, the intracellular delivery of nucleic acids can be measured directly, e.g., using fluorescently labeled nucleic acids. (See, e.g., Bao, et al. (2009) Ann. Rev. Biomed. Engin. 11:25-47; Tyagi, (2009) Nat. Meth. 6(5):331-338; Driks, et al. (2006) Biotechniques 40(4):489-496).

In some embodiments, the intracellular delivery of nucleic acids can be determined indirectly, e.g., by measuring the level of activity of a biologically active nucleic acid (e.g., RNA, RNA interfering agent, cDNA, DNA, or the like) as a function of the efficiency of delivery of the biologically active nucleic acid.

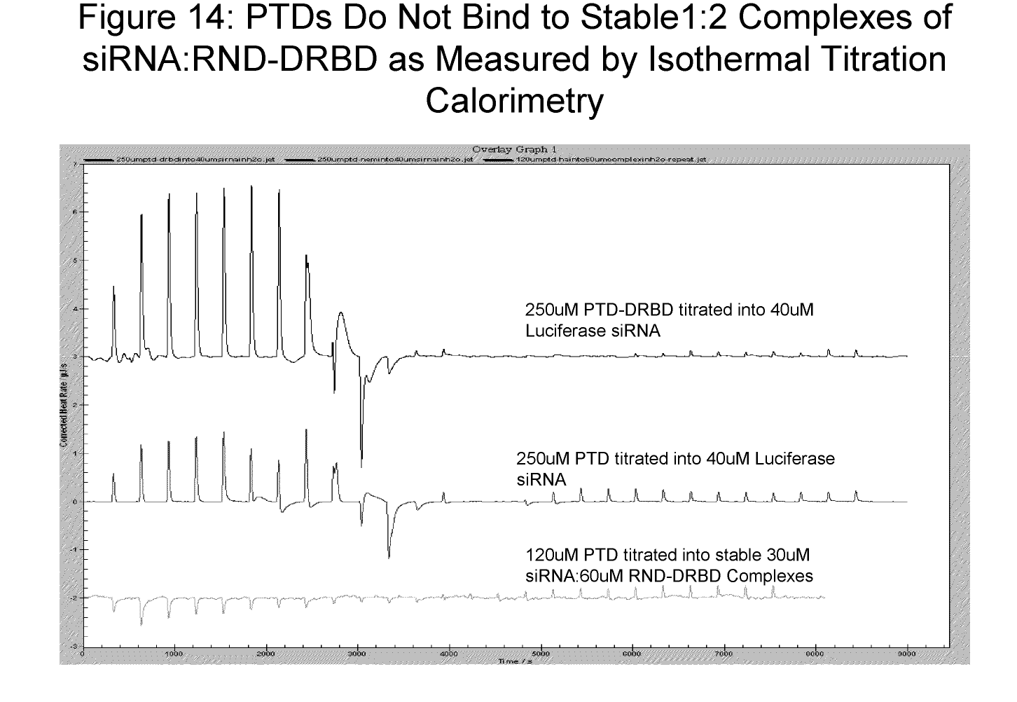

In the embodiments disclosed herein, PTDs, when present in the compositions disclosed herein, are characterized by their ability to facilitate transport of the nucleic acid:RND-DRBD complex across cell membranes as well as having substantially no strong ionic charge-based interaction or association with the nucleic acids of the compositions disclosed herein, or at least substantially less strong, ionic charge-based interactions with the nucleic acids of the compositions herein, when compared to the other components of the compositions, i.e., the RND and DRBD components. For example, the non covalent binding and/or association of PTD with nucleic acids of various thermodynamically stable complexes described herein can be measured using isothermal titration calorimetry, e.g., in differing solutes, such as water, buffered solutions at various pH's and salt/counterion concentrations and the like (See, e.g., Example 3, FIG. 14).

In some embodiments, the PTDs disclosed herein can have substantial alpha-helicity, for example, to optimize transduction of the biomolecule. In another embodiment, the PTD comprises a sequence containing basic amino acid residues that are substantially aligned along at least one face of the peptide. By “substantial” alpha-helicity, it is meant that the circular dichroism (CD) of the peptide show appropriate Cotton effects at key wavelengths. Alpha-helicity of a peptide can be determined by measuring its circular dichroism (CD), and CD data is normally presented as mean residue ellipticies [θ]ₘ. Alpha-helical peptides can show two negative Cotton effects at 208 nm and 222 nm, and a positive Cotton effect at 193 nm, while the CD spectra of peptides with random coil secondary structure are dominated by the increasing negative Cotton effect at shorter wavelength. Alpha-helicity may be estimated from the value at 222 nm, and by comparing the negative Cotton effects at 222 nm and 208 nm. An increasing fraction of [θ]ₘ (222 nm) divided by [θ]ₘ (208 nm) correlates with increasing alpha-helicity. High values for [θ]ₘ (208 nm) compared to [θ]ₘ (222 nm) and a shifting minimum from 208 nm to shorter wavelengths indicate random coil conformation.

In some embodiments, the PTD domain comprises, consists essentially of, or consists of a sequence as in any one of SEQ ID NOs: 1-175.

In some embodiments, the PTD domain comprises a peptide represented by the following general formula: B1-X1-X2-X3-B2-X4-X5-B3, wherein B1, B2, and B3 are each independently a basic amino acid, the same or different; and X1, X2, X3, X4 and X5 are each independently an alpha-helix enhancing amino acid, such as alanine, the same or different.

In some embodiments, the PTD domain comprises a polypeptide represented by the following general formula: X-X-R-X-(P/X)-(B/X)-B-(P/X)-X-B-(B/X), wherein X is any alpha helical promoting residue such as alanine; P/X is either proline or X as previously defined; B is a basic amino acid residue, e.g., arginine (Arg) or lysine (Lys); R is arginine (Arg) and B/X is either B or X as defined herein.

In some embodiments, the PTD can be cationic. For example, in some embodiments, the PTD can include between 7 and 10 amino acids and have the general formula K-X1-R-X2-X1 wherein X1 is R or K and X2 is any amino acid. An example of such a cationic polypeptide can include the sequence RKKRRQRRR (SEQ ID NO:176), or functional fragments and variants thereof.

Other PTDs and CPPs useful in the embodiments disclosed herein include the PTDs and CPPs described in, for example, Langel, Ulo, “Cell Penetrating Peptides, Processes and Applications,” In Langel, Ulo; (Ed.); Handbook of Cell-Penetrating Peptides, 2^{nd} Ed (2007); Langel, Ulo, (Ed.). “Cell-Penetrating Peptides, Mechanisms and Applications;” In Curr. Pharm. Des.; 2005, 11(28)(2005); Langel, Ulo, “Cell-Penetrating Peptides: Processes and Applications” (2002); Wadia, Jehangir S.; Becker-Hapak, Michelle; Dowdy, Steven F. Protein transport. Cell-Penetrating Peptides (2002), pp. 365-375; and S. Deshayes, M. C. Morris, G. Divita and F. Heitz Cell-penetrating peptides: tools for intracellular delivery of therapeutics 2005, V62, N 16, p 1839. each of which is herein incorporated by reference.

Exemplary peptide transduction domains (PTD's) can be derived from the Drosophila homeoprotein Antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), the cationic N-terminal domain of prion proteins, and Buforin II (Park et al. (2000) Proc. Natl. Acad. Sci. U.S.A. 97:8245-50), or the like, or fragments thereof. Yet other exemplary peptide transduction domains are described in International Patent Application Publication No. WO 08/008476, WO 07/095152, WO 03/059940, WO 03/059941, WO 09/041902, WO 05/084158; WO 00/062067, WO 00/034308, and WO 99/55899, U.S. Patent Application Publication No's. 2002/0102265, 2003/0082561, 2002/0102265, and 2003/0040038, the disclosures of which are hereby expressly incorporated by reference in their entireties.

Still other PTDs useful in the embodiments disclosed herein include, but are not limited to those provided in SEQ ID NOs:1-176 Accordingly, PTDs useful in the embodiments described herein include PTDs derived from protamine (AAA39985), penetratin (1OMQ_A), TAT (NP_{—}057853), pVEC, Cationic prion protein domains, P101 (ACT78456), MATa2 (Q6B184), HIV-1 rev (CAA41586), Polyomavirus Vp1 (AAP14004), NF-kappaB (NP_{—}003989), M9 (BAA76626), Vpr (BAH97661), FP_NLS (MPG), Sp-NPS (ACU27162), SN50, Importins and Karyopherins, e.g., Karyopherin alpha (NP 002255), and Karyopherin beta (NP 002256), and the like.

Additional transducing domains useful in the embodiments disclosed herein include but are not limited to a TAT fragment that comprises at least amino acids 49 to 56 of TAT up to about the full-length TAT sequence as described in PCT Pub. No. WO 08/008476. In some embodiments, a TAT fragment can include one or more amino acid changes sufficient to increase the alpha-helicity of the fragment. In some embodiments, amino acid changes are introduced in the PTDs that add a recognized alpha-helix enhancing amino acid. In some embodiments, amino acids are introduced in the PTD's that remove one or more amino acids from the TAT fragment that impede alpha helix formation or stability. In some embodiments, for example, the PTD can be a TAT fragment that includes at least one amino acid substitution with an alpha-helix enhancing amino acid, such as alanine.

Yet additional PTDs useful in the embodiments disclosed herein include a TAT fragment in which the TAT 49-56 sequence has been modified so that at least two basic amino acids in the sequence are substantially aligned along at least one face of the TAT fragment. Exemplary TAT fragments useful as PTDs in the embodiments disclosed herein can include at least one specified amino acid substitution in at least amino acids 49-56 of TAT which substitution aligns the basic amino acid residues of the 49-56 sequence along at least one face of the segment.

In some embodiments, the PTD used in the embodiments disclosed herein can be a recombinant or synthetic PTD designed to mimic and/or enhance the translocating properties of known PTDs, based on consideration of parameters such as electrostatic and hydrophobic properties or secondary structure (Wender et al. (2000) Proc. Natl. Acad. Sci. U.S.A. 97:13003-8; Futaki et al. (2001) J. Biol. Chem. 276:5836-40). An exemplary artificial PTD is transportan (Pooga et al. (1998) FASEB J. 12:67-77; Soomets et al. (2000) Biochim. Biophys. Acta 1467:165-76). Synthetic PTDs such as polylysine, polyarginine, and polyhistidine (which can be positively charged based on the pH of the formulation) e.g., polyarginine (6-15 amino acids) are useful in the embodiments disclosed herein.

In some embodiments, the compositions disclosed herein can include 1, 2, 3, 4, 5, 6, 7, 8, 9, 10, or more PTD domains. In some embodiments, the compositions include PTDs derived from at least two different transducing proteins. For example, chimeric PTDs useful in the embodiments disclosed herein include a chimera between two different TAT fragments, e.g., one from HIV-1 and the other from HIV-2 or one from a prion protein and one from HIV.

In some embodiments, the PTD can interact with the other components of a nucleic acid delivery complex disclosed herein solely by non-covalent interactions. In some embodiments, the PTD(s) of the compositions and complexes disclosed herein can be covalently bound to one or more of the other components of a nucleic acid delivery complex or RNA complex as described herein. For example, in some embodiments, the PTD of the nucleic acid delivery complex can be covalently linked to the nucleic acid, either at the 3′ or 5′ end. Methods for covalently attaching a PTD to a nucleic acid are well-known, and include, but are not limited to, those described in U.S. Patent Application Publication No. 2005/0260756, U.S. Patent Application Publication No. 2002/009758, U.S. Pat. No. 6,025,792, and the like.

In some embodiments, the PTD(s) of the complexes disclosed herein can be covalently linked to an RND or a DRBD. For example, in some embodiments, the PTD can be part of a fusion protein with one or more RND and/or DRBD domains. As discussed below, in some embodiments, the PTD(s) of the complexes disclosed herein can be covalently joined to another functional domain of the composition, e.g., another PTD, an RND, and/or a DRBD, via a chemical crosslinker. In some embodiments, a single PTD can be covalently linked to more than one functional domain, such as another PTD, a DRBD, or an RND. It will be appreciated by the skilled artisan that in the context of the chimeric/fusion proteins disclosed herein, the functional domains can be present in any order.

The PTDs in the embodiments disclosed herein include any of the full length polypeptides of the sequences provided above, or the foregoing accession numbers, as well as fragments or variants thereof, including as modified polypeptides, e.g., including peptide variants that have amino acid substitution(s), e.g., between about 1, 2, 3, 4, 5, 6, 7, 8, 9, 10, 11, 12, 13, 14, 15, 16, 17, 18, 19, 20, 21, 22, 23, 24, 25, or more, amino acid substitutions. In some embodiments, the PTD variants exhibit substantially similar or enhanced biological activity compared to the corresponding unmodified PTD.

### Double-Stranded RNA Binding Domains

The nucleic acid delivery complexes disclosed herein include one or more double stranded RNA binding domain proteins (“dsRBD” or “DRBDs”), also referred to as proteins containing a double stranded RNA binding motif (“dsRBM”). These terms can be used interchangeably. As used herein, the term “double stranded RNA binding domain” refers to a class of polypeptide structures that are approximately 50-100 amino acids in naturally occurring proteins that are responsible for many, although not all interactions of proteins with RNA duplexes. In some embodiments, the DRBD has a characteristic α-β-β-β-α topology that forms an α/β sandwich fold. In some embodiments, the α/β sandwich fold interacts with one face of a dsRNA helix, and spans about 16 bp of the RNA.

Exemplary DRBDs are found within the following proteins: (with Accession numbers listed in parenthesis) include but are not limited to: PKR (AAA36409, AAA61926, Q03963), TRBP (P97473, AAA36765), PACT (AAC25672, AAA49947, NP609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP499172, NP198700, BAB19354), HYL1 (NP563850), hyponastic leaves (CACO5659, BAB00641), human rhinovirus polyprotein (ACT09659), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP059592, CAA59168), RNaseIII (AAF80558, AAF59169, Z81070Q02555/S55784, P05797), and Dicer (BAA78691, AF408401, AAF56056, 544849, AAF03534, Q9884), RDE-4 (AY071926), FLJ20399 (NP060273, BAB26260), CG1434 (AAF48360, EAA12065, CAA21662), CG13139 (XP059208, XP143416, XP110450, AAF52926, EEA14824), DGCRK6 (BAB83032, XP110167) CG1800 (AAF57175, EAA08039), FLJ20036 (AAH22270, XP134159), MRP-L45 (BAB14234, XP129893), CG2109 (AAF52025), CG12493 (NP647927), CG10630 (AAF50777), CG17686 (AAD50502), T22A3.5 (CAB03384) and Accession number EAA14308. As used herein, DRBDs can include any of the full length polypeptides of the foregoing accession numbers, as well as fragments or variants thereof, including as modified polypeptides, e.g., including peptide variants that have amino acid substitution(s), e.g., between about 1, 2, 3, 4, 5, 6, 7, 8, 9, 10, 11, 12, 13, 14, 15, 16, 17, 18, 19, 20, 21, 22, 23, 24, 25, or more, amino acid substitutions. In some embodiments, the modified DRBDs do not exhibit significantly less biological activity, compared to the corresponding unmodified DRBD.

For example, in some embodiments, the nucleic acid delivery complexes and/or the dsRNA complexes and compositions disclosed herein include 1, 2, 3, 4, 5, 6, 7, 8, 9, 10, or more DRBDs. In some embodiments, the nucleic acid:RND-DRBD complexes disclosed herein include more than one of the same DRBD. In some embodiments, the nucleic acid:RND-DRBD complexes disclosed herein include two or more different DRBDs.

In some embodiments, the DRBDs of the nucleic acid:RND-DRBD complexes include one or more cysteine residues, which are either present in the naturally occurring sequences, or present due to site directed mutagenesis and/or amino acid substitution. In some embodiments, the cysteine residues of the DRBD can be used in crosslinking the DRBD to another component of the nucleic acid delivery complex, such as an RND, PTD, or the like. In some embodiments, the DRBDs of the nucleic acid:RND-DRBD complexes include one or more serine residues, which are either present in the naturally occurring sequences, or present due to site directed mutagenesis and/or amino acid substitution. In some embodiments, the serine residues of the DRBD can be used in crosslinking the DRBD to another component of the nucleic acid delivery complex, such as an RND, PTD, or the like. In some embodiments, the DRBD(s) of the nucleic acid:RND-DRBD complexes include one or more lysine residues which are either present in the naturally occurring sequences, or present due to site directed mutagenesis and/or amino acid substitution. In some embodiments, the lysine residues of the DRBD can be used in crosslinking the DRBD to another component of the nucleic acid delivery complex, such as an RND, PTD, or the like. In some embodiments, the DRBD(s) of the nucleic acid:RND-DRBD complexes include one or more aspartic acid residues and/or glutamic acid residues which are either present in the naturally occurring sequences, or present due to site directed mutagenesis and/or amino acid substitution. In some embodiments, the aspartic acid residues and/or glutamic acid residues of the DRBD can be used in crosslinking the DRBD to another component of the nucleic acid delivery complex, such as an RND, PTD, or the like. In some embodiments, the DRBD(s) of the nucleic acid:RND-DRBD complexes include one or more tyrosine residues and/or arginine residues which are either present in the naturally occurring sequences, or present due to site directed mutagenesis and/or amino acid substitution. In some embodiments, the tyrosine residues and/or arginine residues of the DRBD can be used in crosslinking the DRBD to another component of the nucleic acid delivery complex, such as an RND, PTD, or the like. Preferably, any synthetic crosslinks formed between a DRBD and another component of the nucleic acid delivery complex do not substantially alter the binding properties of the DRBD to the nucleic acid of the complex.

Non-limiting examples of DRBDs useful in the embodiments disclosed herein include, but are not limited to:

DPAGDLSAGFFMEELNTYRQKQGVVLKYQELPNSGPPHDRRFTFQ VIIDGREFPEGEGRSKKEAKNAAAKLAVEILNKEKKAAALE (SEQ ID NO:177). In some embodiments, the DRBD can contain one or more amino acid substitutions, such as GDPAGDLSAGFFMEELNTYRQKQGVVLKYQELPNSGPPHDRRFTFQVIIDGREFPEG EGRSKKEAKNAAAKLAVEILNKEKKCAALE (SEQ ID NO:178); GDPAGDLSAGFFMEELNTYRQKQGVVLKYQELPNSGPPHDRRFTFQVIIDGREFPEG EGRSKKEAKNAAAKLAVEILNKEKKACALE (SEQ ID NO:179); GDPAGDLSAGFFMEELNTYRQKQGVVLKYQELPNSGPPHDRRFTFQVIIDGREFPEG EGRSKKEAKNAAAKLAVEILNKEKKAACLE (SEQ ID NO:180); GDPAGDLSAGFFMEELNTYRQKQGVVLKYQELPNSGPPHDRRFTFQVIIDGREFPEG EGRSKKEAKNAAAKLAVEILNKEKKAAALEC (SEQ ID NO:181); GDPAGDLSAGFFMEELNTYRQKQGVVLKYQELPNSGPPHDRRFTFQVIICGREFPEGE GRSKKEAKNAAAKLAVEILNKEKKAAALE (SEQ ID NO:182); GDPAGDLSAGFFMEELNTYRQKQGVVLKYQELPNSGPPHDRRFTFCVIIDGREFPEGE GRSKKEAKNAAAKLAVEILNKEKKAAALE (SEQ ID NO:183); GDPAGDLSAGFFMEELNTYRQKQGVVLKYQELPNSGPPHDRRFTFQVCIDGREFPEG EGRSKKEAKNAAAKLAVEILNKEKKAAALE (SEQ ID NO:184); GDPAGDLSAGFFMEELNTYRQKQGVVLKYQELPNSGPPHDRRFTFQVIIDGCEFPEG EGRSKKEAKNAAAKLAVEILNKEKKAAALE (SEQ ID NO:185); GDPAGDLSAGFFMEELNTYRQKQGVVLKYQELPNSGPPHDRRFTFQVIIDGRECPEG EGRSKKEAKNAAAKLAVEILNKEKKAAALE (SEQ ID NO:186); GDPAGDLSAGFFMEELNTYRQKQGVVLKYQELPNSGPPHDRRFTFQVIIDGREFPGE GRSKKEAKNAAAKLAVEILNKEKKAAALE (SEQ ID NO:187) GDPAGDLSAGFFMEELNTYRQKQGVVLKYQELPNSGPPHDRRFTFQVIIDGREFPEG EGRSKKEAKNAAAKLAVEILCKEKKAAALE (SEQ ID NO:188); GDPAGDLSAGFFMEELNTYRQKQGVCLKYQELPNSGPPHDRRFTFQVIIDGREFPEGE GRSKKEAKNAAAKLAVEILNKEKKAAALE (SEQ ID NO:189); GDPAGDLSAGFFMEELNTYRQKQGVVLCYQELPNSGPPHDRRFTFQVIIDGREFPEGE GRSKKEAKNAAAKLAVEILNKEKKAAALE (SEQ ID NO:190); and GDPAGDLSAGFFMEELNTYRQKQGVVLKYCELPNSGPPHDRRFTFQVIIDGREFPEGE GRSKKEAKNAAAKLAVEILNKEKKAAALE (SEQ ID NO:191).

### Targeting Domains

In some embodiments, the compositions disclosed herein include a targeting domain. For example, in some embodiments, one or more targeting domains can be covalently linked to an RND and/or a DRBD and/or a PTD.

As used herein the term “targeting domain” refers to a domain (e.g., polypeptide, glycopeptide, sugar, or other) that targets the nucleic acid delivery complexes disclosed herein to a specific target cells. As such, targeting domains can be a receptor or receptor ligand that has a cognate binding domain present on the specific target cell. For example, in some embodiments, the nucleic acid delivery complexes can include an antibody or antibody fragment (e.g., an Fc domain of an antibody) that specifically binds to a cognate cell surface antigen. By way of example, a targeting domain such as a growth-factor can be used to target the nucleic acid delivery complex to a target cell having a cognate receptor (e.g., a he heregulin-a₁ isoform targeting domain can be used to target nucleic acid delivery complexes to HER2/3 expressing cells; a basic fibroblast growth factor targeting domain can target nucleic acid delivery complexes to FGF receptor bearing cells; mannose or mannose derivatives can be used to target nucleic acid delivery complexes to dendritic cells, or other cells expressing receptors related to the mannose receptor; steroid targeting domains can be used to target nucleic acid delivery complexes to steroid-receptor bearing cells, and the like. See, e.g., Jeyarajan, et al. (2010) Int. J. Nanomedicine 2010:725-733; Hoganson, et al. (1998) Hum. Gene Ther. 9(17):2565-2575; Diebold, et al. (2002) Somatic Cell Mol. Genet. 27:65-74; Rebuffat, et al. (2001) Nature Biotech. 19:1155-116, Rojanasskaul, et al. (1994) Pharm. Res. 11(12): 1731-1736.

### RNA Masking Agents

In some embodiments, the nucleic acid delivery complexes include an RNA masking agent. As used herein, the term RNA masking agent refers to an agent (polymer or copolymer, or the like) that associates strongly with nucleic acids, e.g., RNA (such as RNA interfering agents), and functions to prevent or inhibit charge associations between a PTD and the RNA interfering agent.

By way of example, in some embodiments, the RNA masking agent can be, e.g., nonionic alkyl poly(ethylene oxide) (PEO), poly(propylene oxide) (PPO), polyethylene glycol (PEG), dioctyl itaconate (DOI)/dioxypropylated itaconic acid copolymers, polymers and copolymers of ethyloxirane, acrylamide, gycerol, vinyl alcohol, vinylpyrrolidone, vinylpyridine, vinylpyridine N-oxide, oxazoline, or acryloyl morpholine, and derivatives thereof, and the like.

### Chimeric Proteins

In some embodiments, the nucleic acid delivery complexes disclosed herein can include a chimeric protein. Chimeric proteins, or fusion proteins, or chimeric peptides, or fusion peptides, as discussed in further detail below. As used herein, the terms “chimeric protein”, “fusion protein”, “chimeric peptide” and “fusion peptide” are interchangeable, and refer to polypeptides which contain two or more functionally distinct regions, each made up of at least one functional monomer unit, e.g., a RNA neutralizing domain, a double stranded RNA binding domain, a protein transduction domain, or the like.

The skilled artisan will appreciate that the functionally distinct regions of the chimeric proteins disclosed herein can be organized in nearly any fashion provided that the construct has the function for which it was intended (e.g., with respect to dsRNA binding and/or charge neutralization, cell transport, and the like).

Each of the functionally distinct regions of the chimeric proteins disclosed herein (e.g., RND(s), DRBD(s), PTD(s), and the like) may be directly linked or may be separated by a linker. In some embodiments, the distinct regions are linked at their C-terminal or N-terminal ends. In some embodiments, a link between two distinct regions is in between the N-terminal and C-terminal amino acid residues of the different domains. By way of example, one domain can contain an amino acid residue between the N-terminus and C-terminus that is crosslinker-reactive, and through which another domain is joined. The domains may be presented in any order. Additionally, the chimeric or fusion proteins or polypeptides can include tags, e.g., to facilitate identification and/or production and/or purification of the fusion protein or polypeptide, such as a 6×HIS tag, a maltose binding protein domain, a GST tag, or the like.

In some embodiments, the compositions described herein include a peptide linker. For example, in some embodiments, a peptide linker comprises up to about 20 or 30 amino acids, commonly up to about 10 or 15 amino acids, and still more often from about 1 to 5 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, the amino acid sequence of the linker is engineered to be flexible so as not to hold the fusion molecule in a single rigid conformation. Peptide linker sequences can be used, e.g., to space the DRBD(s), the RND(s), PTDs and other functional domains of the chimeric proteins disclosed herein from each other. For example, a peptide linker sequence can be positioned between a RND and/or DRBD and/or a PTD, e.g., to provide molecular flexibility. The length of the linker moiety can be chosen to optimize the biological activity of the chimeric protein and can be determined empirically without undue experimentation. Exemplary peptide linkers and linker moieties are described in Int. Pub. No. WO 08/008476, Huston et al., Proc. Natl. Acad. Sci. 85:5879, 1988; Whitlow et al., Protein Engineering 6:989, 1993; and Newton et al., Biochemistry 35:545, 1996. Other suitable peptide linkers are those described in U.S. Pat. Nos. 4,751,180 and 4,935,233

The chimeric proteins disclosed herein can be made using routine and well-established recombinant biology techniques (see, e.g. Sambrook et al., Molecular Cloning: A Laboratory Manual, New York: Cold Spring Harbor Press, 1989).

Also provided herein are vectors encoding the recombinant chimeric proteins disclosed herein, as well as host cells including nucleic acids that encode the chimeric proteins disclosed herein.

Some embodiments provide isolated nucleic acids that encode the recombinant proteins described herein. An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

The chimeric polypeptides, domains (e.g., RND, DRBD, PTDs) or subunits of domains (e.g., an RND subunit) described herein can be produced in recombinant host cells following conventional techniques. To express a recombinant polypeptide, a nucleic acid molecule encoding the polypeptide is operably linked to regulatory sequences that controls transcriptional expression in an expression vector and then introduced into a host cell. In addition to transcriptional regulatory sequences, such as promoters and enhancers, expression vectors can include translational regulatory sequences and a marker gene, which is suitable for selection of cells that carry the expression vector.

Expression vectors that are suitable for production of a foreign protein in eukaryotic cells useful in the embodiments disclosed herein can contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. The expression vectors in the embodiments disclosed herein can also include nucleotide sequences encoding a secretory sequence that directs the heterologous polypeptide into the secretory pathway of a host cell. For example, the expression vector can comprise a coding sequence of a domain (RND, DRBD, PTD) or domain subunit and a secretory sequence.

In some embodiments, the recombinant polypeptides described herein can be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., (1986) Som. Cell. Molec. Genet. 12:555), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650), murine embryonic cells (NIH-3T3; ATCC CRL 1658) and the like.

For a mammalian host, the transcriptional and translational regulatory signals can be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., (1982) J. Molec. Appl. Genet. 1:273), the TK promoter of Herpes virus (McKnight, (1982) Cell 31:355), the SV40 early promoter (Benoist et al., Nature 290:304 (1981)), the Rous sarcoma virus promoter (Gorman et al., (1982) Proc. Nat'l Acad. Sci. USA 79:6777), the cytomegalovirus promoter (Foecking et al., (1980) Gene 45:101), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control the expression of the recombinant polypeptides in mammalian cells if the prokaryotic promoter is regulated by a eukaryotic promoter (Zhou et al., (1990) Mol. Cell. Biol. 10:4529, and Kaufman et al., (1991) Nucl. Acids Res. 19:4485).

Expression vectors can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

By way of example, example, a selectable marker can comprise a gene that provides resistance to an antibiotic, such as neomycin or the like. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Recombinant polypeptides disclosed herein can also be produced by cultured mammalian cells using a viral delivery system. Exemplary viruses for this purpose include adenovirus, herpesvirus, vaccinia virus and adeno-associated virus (AAV). (See, e.g., Becker et al., (1994) Meth. Cell Biol. 43:161, and Douglas and Curiel, (1997) Science & Medicine 4:44).

The recombinant polypeptides disclosed herein can also be expressed in other higher eukaryotic cells, such as avian, fungal, insect, yeast, or plant cells. By way of example, in some embodiments, a baculovirus expression system is used to express the recombinant polypeptides disclosed herein, e.g., the BAC-to-BAC kit (Life Technologies, Rockville, Md.). Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

In some embodiments, the recombinant polypeptides disclosed herein can be expressed in fungal cells, including yeast cells such as Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, e.g., Gleeson et al., (1986) J. Gen. Microbiol. 132:3459, and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. In some embodiments, the recombinant polypeptides disclosed herein can be produced in Pichia methanolica host cells, as described, e.g., by Raymond, U.S. Pat. No. 5,716,808, Raymond, U.S. Pat. No. 5,736,383, Raymond et al., (1998) Yeast 14:11-23, and in international publication Nos. WO 97/17450, WO 97/17451, WO 98/02536, and WO 98/02565.

In some embodiments, the recombinant polypeptides disclosed herein can be expressed in prokaryotic host cells. Suitable promoters that can be used to express the recombinant polypeptides in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. (1987) 1:277, Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH4I, DHS, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)).

When expressing a recombinant polypeptide disclosed herein in bacteria such as E. coli, the polypeptide may be retained in the cytoplasm, typically as insoluble granules, or may be directed to the periplasmic space by a bacterial secretion sequence. In the former case, the cells can be lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution according to routine techniques. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et al. (eds.), Chapter 4, starting at page 101 (John Wiley & Sons, Inc. 1996), and Rudolph, “Successful Refolding on an Industrial Scale”, Chapter 10).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

In some embodiments, one or more domains of the chimeric proteins disclosed herein can be produced in a recombinant expression system as described above, or synthesized using customary peptide synthesis protocols as described herein, and covalently linked together. For example, in some embodiments, one or more recombinantly produced domains (e.g., RND, PTD, DRBD, Targeting domain, RND subunit) is covalently joined to one or more other domains using conventional crosslinkers. In some embodiments, one or more chemically synthesized domains (e.g., RND, PTD, DRBD, Targeting domain, RND subunit) is covalently joined to one or more other domains using conventional crosslinkers. Crosslinking reactions useful in the embodiments disclosed herein include but not limited to, disulfide formation, free thiol bromoacetyl reactions, free thiol maleimde reactions, azide alkynyl addition reactions (e.g. Click chemistry, Huisgen reaction), via homo-bifunctional linkers, hetero-bifunctional linkers and the like. Exemplary, non limiting, reactive groups in a chemical cross-linking reagents useful in the embodiments disclosed herein can belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), NHS-3-maleimidopropionate (MPS), etc. For example, carbodiimide-mediated amide formation and active ester maleimide-mediated amine and sulfhydryl coupling are widely used crosslinking approaches useful in the embodiments disclosed herein.

In some embodiments, the joining of two functional domains can include the coupling of a thiol group on one domain to a thiol group on a second domain, e.g., with a homobifunctional crosslinker such as bis-maleimidoethane; bis-maleimidohexane; 1,4-bis-maleimidyl-2,3-dihydroxybutane; bis-maleimdo (PEG)ₙ crosslinkers, where n=1-10; or 1,4-bis-maleimidobutane, or the like.

In some embodiments, the joining of two functional domains can include the coupling of an amine group on one domain to a thiol group on a second domain, sometimes by a two- or three-step reaction sequence. A thiol-containing molecule can be reacted with an amine-containing molecule using a heterobifunctional cross-linking reagent, e.g., a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., can be used. For example, amines can be indirectly thiolated by reaction with 2-Iminothiolane (Traut's reagent) or succinimidyl acetylthioacetate (SATA) followed by removal of the acetyl group with, for example, hydroxylamine or hydrazine, using conventional methods.

Amine-carboxylic acid crosslinking 1-Ethyl-3-(3-dimethylaminopropyl)carbodiimide (EDAC, E2247) can react with biomolecules to form “zero-length” crosslinks, usually within a molecule or between subunits of a protein complex. In this chemistry, the crosslinking reagent is not incorporated into the final product. The water-soluble carbodiimide EDAC crosslinks a specific amine and carboxylic acid between subunits of allophycocyanin, thereby stabilizing its assembly. Reaction of carboxylic acids with cystamine and EDAC followed by reduction with DTT results in thiolation at carboxylic acids. This indirect route to amine-carboxylic acid coupling is particularly suited to acidic proteins with few amines, carbohydrate polymers, heparin, poly(glutamic acid) and synthetic polymers lacking amines.

Additional general information relating to conjugation methods and cross-linkers can be found, for example, in Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong S S, Chemistry of Protein Conjugation and Cross-linking, CRC Press Publishers, Boca Raton, 1991; and Hermanson, G. T., Bioconjugate Techniques, Academic Press, Inc., San Diego, 1996.

In some embodiments, the chimeric proteins are arranged as follows: RND-DRBD-PTD. In some embodiments, the chimeric proteins further include 1-5 PTDs covalently linked to the DRBD. In some embodiments, the PTDs are covalently linked to the sulfhydryl groups present in cysteine residues in the DRBD domain. For example, in some embodiments, the PTDs are chemically joined to the DRBD domain of an RND-DRBD recombinant polypeptide by a maleimide crosslinker.

Example 4, below, describes the crosslinking of an exemplary PTD to an exemplary RND-DRBD fusion protein to generate the stable complexes described herein.

Exemplary, non-limiting examples of various arrangements of fusion proteins useful in the embodiments disclosed herein include, but are not limited to, (RND)ₓ-(DRBD)_{y}; (DRBD)ₓ-(RND)_{y}; (PTD)ₓ-(RND)_{y}-(DRBD)_{z}; (RND)ₓ-(DRBD)_{y}-(PTD)_{z}; (PTD)ₓ-(DRBD)_{y}-(RND)_{z}; (DRBD)ₓ-(RND)_{y}-(PTD)_{z}, and the like, where “-” indicates a covalent linkage and “x, y and z” are greater than or equal to one (1) and “(RND)” is a complete RND when x=1, and a unit of an RND for x greater than one. Preferred embodiments comprise the following composition, RND-DRBD-PTD. Any of the foregoing fusion proteins can include a linker, as described herein, at the junction between two functional domains. Furthermore, any of the components of the fusion protein can be made either synthetically or recombinantly.

### Nucleic Acid Delivery Complexes

Some embodiments herein provide compositions comprising nucleic acid delivery complexes. Each of the “nucleic acid delivery complexes” include, at least, a chimeric protein as described above (e.g. an RND-DRBD-PTD) noncovalently bound to a nucleic acid (e.g. dsRNA). In some embodiments the nucleic acid delivery complex has biological activity (e.g. the ability to transport across a cell membrane, mediating intracellular delivery of the nucleic acid, and/or, in the case where the nucleic acid is an RNA interfering agent silencing a target gene or target genes of interest).

Methods of the nucleic acid delivery complexes are also herein. In some embodiments, the nucleic acid delivery complexes disclosed herein can be made by mixing a nucleic acid, e.g., an RNA interfering agent, with a DRBD and an RND and a PTD.

In some embodiments, the molar ratio of nucleic acid e.g., RNA interfering agent to RND within the complex can be 1:0.1, 1:1, 1:2, 1:3, 1:4, 1:5, 1:6, 1:7, 1:8, 1:9, 1:10, or more, or any fraction in between. In preferred embodiments, the ratio of nucleic acids, e.g., RNA interfering agents:RND in the compositions provided herein is between about 0.1:1 to about 1:4, e.g., 1:2. In some embodiments, the molar ratio of nucleic acid (e.g., RNA interfering agent) to DRBD within the complex can be 1:0.1, 1:1, 1:2, 1:3, 1:4, 1:5, 1:6, 1:7, 1:8, 1:9, 1:10, or more, or any fraction in between. In preferred embodiments, the ratio of the nucleic acids, e.g., the RNA interfering agents such as dsRNA to DRBD is about 1:0.1 to about 1:4, e.g., about 1:2. In some embodiments, the compositions disclosed herein include 1, 2, 3, 4, 5, 6, 7, 8, 9, 10, or more PTDs. For example, in some embodiments, the molar ratio of nucleic acid (e.g., RNA interfering agent) to PTD within the complex can be 1:0.1, 1:1, 1:2, 1:3, 1:4, 1:5, 1:6, 1:7, 1:8, 1:9, 1:10, 1:20, or more, or any fraction in between.

In some embodiments, the molar ratio of DRBD to RND within the complexes disclosed herein can be 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, or more, or any fraction in between. In some embodiments, the molar ratio of PTD to RND within the complexes disclosed herein can be 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, or more, or any fraction in between. In some embodiments, the molar ratio of PTD to DRBD within the complexes disclosed herein can be 0.1:1, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, or more, or any fraction in between.

In some embodiments, the nucleic acid, e.g., RNA interfering agent, and the RND and/or DRBD can be mixed in an unbuffered or buffered aqueous solution. In some embodiments, the mixture can have a pH of between about 3-9. In some embodiments, the mixture can be a buffer and/or mixture of ions comprising ammonium, sodium, potassium, chloride, magnesium, phosphate, carbonate, acetate, citrate, etc., or the like, at different strengths. In some embodiments, the nucleic acid, e.g., RNA interfering agent, and the RND and/or DRBD can be mixed in an unbuffered or buffered aqueous solution also containing surfactants and/or cosolvents and/or stabilizers and/or tonicity adjusting agents and/or preservatives and/or nonpolar modifiers and/or other complexing agents and/or reaction catalysts.

Upon mixing, the RND and DRBD cooperatively bind to the nucleic acid, e.g., RNA interfering agent, to form a nucleic acid:RND-DRBD complex, wherein the molar ratio of the nucleic acid:RND-DRBD is between about 1:1 to 1:5 (e.g. about 1:2). In some embodiments, the mixture is allowed to incubate to rearrange into a thermodynamically stable state for at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 3 days, 4 days, 5 days, 6 days, a week, or longer.

In some embodiments, the nucleic acid:RND-DRBD complex is a thermodynamically stable, low energy state complex. Without being bound to a particular theory, it is believed that in some embodiments, the DRBD is bound to the functional groups present in the major and minor grooves of the nucleic acid, e.g., dsRNA, through hydrogen bonds and non-polar attractive forces, and the RND is bound to the nucleic acid, e.g., dsRNA via strong, ionic charge-based interactions. In some embodiments, the thermodynamically stable complex of the nucleic acid RND-DRBD is evidenced by a high transition temperature of complex dissociation as measured, e.g., by art-accepted techniques such as differential scanning calorimetry. For example, in some embodiments, the thermodynamically stable nucleic acid:RND-DRBD exhibits a transition temperature of complex dissociation at or above 90° C., and greater than that observed for the unwinding of the strands of non complexed dsRNA (Tₘ for dsRNA). In some embodiments, the thermodynamically stable complex of the nucleic acid:RND-DRBD complex is evidenced by a substantially neutral zeta potential. In some embodiments, the nucleic acid:RND-DRBD complex is substantially neutralized, and further charge-based interactions with the dsRNA do not occur.

In some embodiments, one or more PTDs can be added to the nucleic acid. In some embodiments, the PTD can be added prior to, or simultaneously with the RND(s), and/or DRBD(s). For example, in some embodiments, the PTD(s) are added to the nucleic acid with the RND and DRBD by virtue of being a fusion protein with a DRBD and/or an RND.

In some embodiments, the PTD(s) are activated with a crosslinker prior to adding the PTD to the mixture. For example, in some embodiments, an activated PTD can be added to the mixture, thereby joining the PTD to a reactive group (e.g., a thiol group, a hydroxyl group, an amine group, or a carboxylic acid group) on RND and/or a DRBD. In some embodiments, an activated PTD can be added to the mixture, thereby joining the PTD to the 5′ or 3′ end of the nucleic acid, e.g., RNA interfering agent which is non-covalently bound to the RND and/or DRBD.

In some embodiments, the PTD can be added subsequent to mixing the nucleic acid:RND-DRBD complex. For example, in some embodiments, the PTD can be added after the RND, DRBD and nucleic acid form a strong, tightly bound, thermodynamically stable nucleic acid:RND-DRBD complex as described above, e.g., after the RND, DRBD and RNA interfering agent reach binding equilibrium. For example, in some embodiments, the PTD can be added to the mixture at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 3 days, 4 days, 5 days, 6 days, a week, 2 weeks, 3 weeks 4 weeks, 5 weeks 6 weeks, 7 weeks, 8 weeks, 3 months, or longer, after the nucleic acid and RND-DRBD are mixed. For example, in some embodiments an activated PTD can be added to the mixture, thereby joining the PTD to a reactive group (e.g., a thiol group, a hydroxyl group, an amine group, or a carboxylic acid group, among others) on RND and/or a DRBD that is already in a nucleic acid:RND-DRBD complex that is in a low energy state and that is thermodynamically stable.

By way of example, in some embodiments the nucleic acid delivery complexes can be made by (1) providing a RND-DRBD chimera (e.g., a chimeric protein that includes an RND and a DRBD, wherein the chimera is engineered such that the DRBD includes one or more reactive groups, e.g., 1, 2, 3, or 4 cysteine residues; (2) mixing the RND-DRBD chimera with the nucleic acid (e.g., an siRNA of interest), and incubating the mixture for at least 1 hour, to form a thermodynamically stable nucleic acid:RND-DRBD complex as described herein; (3) providing a PTD that includes at least one reactive functional group (e.g., cysteine, or the like) and that that has been activated with a linker (e.g., maleimide), that forms crosslinks between reactive groups; and (4) mixing the activated PTD with the thermodynamically stable nucleic acid:RND-DRBD-complex to form a biologically active nucleic acid:RND-DRBD-PTD complex.

In some embodiments, the nucleic acid delivery complexes can be generated by (1) providing a RND-DRBD, wherein the chimera is engineered such that the DRBD includes one or more reactive groups, such as 1, 2, 3, or 4 cysteine residues; (2) providing a PTD having a reactive functional group (e.g., a cysteine) that been activated with a linker (e.g., maleimide), that forms crosslinks between reactive groups; (3) mixing the activated PTD with the RND-DRBD chimera, to form an RND-DRBD-PTD chimera; and (4) mixing the RND-DRBD-PTD chimera with a nucleic acid, e.g., an siRNA, to form a biologically active nucleic acid delivery complex

### Formulations

Provided herein are pharmaceutical compositions which include the complexes described herein and one or more pharmaceutically acceptable excipients. The term “pharmaceutically acceptable” refers to a formulation of a compound that does not cause significant irritation to a subject to which it is administered and does not abrogate the biological activity and properties of the compound.

Also provided are methods of making a pharmaceutical formulation of a nucleic acid, such as an RNA interfering agent. In some embodiments, the method includes the step of forming a thermodynamically stable complex of one or more RND(s), DRBD(s) and the nucleic acid, e.g., RNA interfering agent, then adding an activated PTD to the thermodynamically stable complex such that a covalent linkage is formed between the PTD and either the RND, DRBD or RNA interfering agent as described herein, and storing the thermodynamically stable complex for a period of time, e.g., greater than a week, 2 weeks, a month, several months, a year or longer. In some embodiments, the pharmaceutical formulation retains activity at least for the period of time indicated, e.g., the pharmaceutical formulation provides substantially similar levels intracellular delivery of the nucleic acid (e.g., a biologically active RNA interfering agent) over time, as determined by the methods described herein.

The pharmaceutical compositions may be administered in a number of ways depending upon whether local or systemic treatment is desired and upon the area to be treated. Administration may be topical (including ophthalmic, dermal, and to mucous membranes including buccal, vaginal and rectal delivery), pulmonary (e.g., by inhalation or insufflations of powders or aerosols, including by a nebulizer, dry powder inhaler, or metered dose inhaler); intratracheal, intranasal, epidermal and transdermal, oral or parenteral. Parenteral administration includes intravenous, intraarterial, subcutaneous, intraperitoneal, intraarticular or intramuscular injection or infusion; or intracranial, (e.g., intrathecal or intraventricular) administration. Compounds with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical preparations disclosed herein can be used in combination with a drug delivery device. Coated condoms, catheters, gloves, other medical devices, and the like, may also be useful.

The pharmaceutical formulations, which may conveniently be presented in unit or multi-unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In some embodiments, the compositions disclosed herein can be formulated as suspensions (colloidal or otherwise) in aqueous, non-aqueous or mixed media. Aqueous suspensions can contain substances which increase the viscosity of the suspension including, for example, glycerol, polyethylene glycol, propylene glycol, sodium carboxymethylcellulose, sorbitol, dextran and the like. The suspension may also contain stabilizers.

Pharmaceutical compositions include, but are not limited to, solutions, emulsions, foams and liposome-containing formulations. The pharmaceutical compositions and formulations may comprise one or more penetration enhancers, carriers, excipients or other active or inactive ingredients.

Emulsions are typically heterogeneous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. In some embodiments, the compositions disclosed herein are formulated as microemulsions. Emulsions and their uses are well known in the art and are further described in U.S. Pat. No. 6,287,860, incorporated herein by reference in its entirety.

In some embodiments, the pharmaceutical formulations and compositions disclosed herein can include one or more surfactants. The use of surfactants in drug products, formulations and in emulsions is well known in the art. Surfactants and their uses are further described, e.g., in U.S. Pat. No. 6,287,860, which is incorporated by reference in its entirety.

In one embodiment, various penetration enhancers are employed to affect the efficient delivery of the compositions disclosed herein. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated by reference in its entirety.

One of skill in the art will recognize that formulations are routinely designed according to their intended use, i.e. route of administration.

Preferred formulations for topical administration include those in which the compounds disclosed herein are in admixture with a topical delivery agent such as lipids, fatty acids, fatty acid esters, steroids, chelating agents and surfactants. Preferred lipids include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA) compounds.

In some embodiments, the compositions disclosed herein are provided as topical formulations. Non-limiting examples of topical formulations useful in the embodiments disclosed herein are described in detail in U.S. Pat. No. 6,887,906, which is incorporated herein by reference in its entirety.

In some embodiments, the compositions disclosed herein can be formulated for oral administration. Preferably, the oral formulations disclosed herein are sufficiently protective of the complex to prevent digestion and to allow entry into tissue. Oral forms useful in the embodiments disclosed herein can include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein by reference in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include, but are not limited to, polyoxyethylene-9-lauryl ether, polyoxyethylene-20-cetyl ether.

The compositions disclosed can be formulated for oral delivery, for example, in granular form including sprayed dried particles, or complexed to form micro or nanoparticles. Complexing agents and their uses are further described in U.S. Pat. No. 6,287,860, which is incorporated herein in its entirety. Oral formulations for oligonucleotides and their preparation are described in detail in U.S. Pat. No. 6,887,906, U.S. Pat. No. 6,747,014, and U.S. Patent Application Publication No. US 2003-0027780, each of which is incorporated herein by reference in its entirety.

Compositions and formulations for parenteral, intrathecal or intraventricular administration can include sterile aqueous solutions which may also contain buffers, diluents and other suitable additives such as, but not limited to, penetration enhancers, carrier compounds and other pharmaceutically acceptable carriers or excipients.

In some embodiments, the compositions can contain one or more different species of complexes disclosed herein. For example, in some embodiments, the compositions can include a first species of a nucleic acid complex that comprises an RNA interfering agent specific for a first target nucleic acid and one or more additional species of complexes that each include RNA interfering agents specific for a second nucleic acid target, third nucleic acid target, fourth nucleic acid target, or the like. By way of example, in some embodiments, the compositions disclosed herein can include two or more species of nucleic acid complexes wherein the RNA interfering agents of the different complexes target different genes that are implicated in a particular disease state, such as cancer (see, e.g., Pandyra, et al. (2007) J. Pharmacol. Exp. Ther. 322(1):123-132), or different genes implicated in a single physiological condition, e.g., angiogenesis. See, e.g., Lu, et al. (2005) Trends Mol. Med. 11(3):104-113

In some embodiments, the compositions can contain one or more species of complexes disclosed herein, wherein the two or more species comprise two or more RNA interfering agents targeted to different regions of the same nucleic acid target. By way of example, in some embodiments, the compositions include complexes that include RNA interfering agents that target the same or different regions (e.g., coding regions) of viral nucleic acids. See, e.g., Chen, et al. (2008) J. Drug Target. 16(2):14-148, Xin, et al. (2008) Hepatogastronenterology, 55(88):2178-2183, Fulton et al. (2009) PLoS One 4(1) e4118.

In some embodiments, compositions are provided that comprise a mixture of more than one complex as disclosed herein, e.g., a mixture of thermodynamically stable RNA interfering agent:RND-DRBD or biologically active nucleic acid delivery complexes. In some embodiments, the different complexes are not mixed together. For example, in some embodiments, the two or more complexes can be used sequentially.

Compositions of the present invention containing any of the complexes described herein can be filled into sterile vials, containers, and the like. By way of example, in some embodiments, the compositions disclosed herein can be in a liquid or lyophilized form in a septum-sealed container. In some embodiments, thermodynamically stable siRNA:RND-DRBD complexes can be in a liquid or lyophilized form, and an activated PTD in either liquid or lyophilized form (e.g., either in the same or opposite state as the siRNA:RND-DRBD stable complex), wherein the septum-sealed container separates the thermodynamically stable complex from the activated PTD, allowing for mixing and the formation of the biologically active nucleic acid delivery complex just prior to dosing. In some embodiments is the case whereby a septum-sealed container separates a nucleic acid in either liquid or lyophilized form from a chimeric polypeptide, i.e., a RND-DRBD-PTD fusion protein in the same or opposite physical state, allowing for mixing and the formation of the biologically active nucleic acid complex just prior to dosing. This type of container system is useful where the biologically active nucleic acid complex is physically and/or chemically stable for only a brief period of time. All of the above described container/closures can be either multi- or unit dosage forms.

### Methods of Delivering Nucleic Acids to Cells

Also provided herein are methods of delivering nucleic acids into target cells, using the compositions disclosed herein, by contacting the target cell with the compositions. In some embodiments, the contacting can be in vitro. In some embodiments, the contacting can be in vivo.

As used herein, the term “target cell” can refer to a eukaryotic cell, a plant cell, a fungal cell, or a prokaryotic cell. In preferred embodiments, the target cell is a eukaryotic cell, such as a mammalian cell. Non-limiting examples of different types of eukaryotic “target cells” useful in the embodiments disclosed herein include, for example, antigen-presenting cells, dendritic cells neural cells, such as brain cells, astrocytes, microglial cells, and the like, spleen cells, lymphoid cells, lung cells, skin cells, keratinocytes, endothelial cells, such as vascular endothelial cells, colonic epithelium cells, lung epithelium cells and the like, alveolar cells, such as alveolar macrophages, alveolar pneumocytes, and the like, mesenchymal cells, hematopoietic bone marrow cells, adipocytes, cardiac myocytes, and the like.

Other target cells useful in the embodiments disclosed herein include a Claudius' cell, Hensen cells, Merkel cells, Muller cells, Paneth cells, Purkinje cells, Schwann cells, Sertoli cells, acidophil cells, acinar cells, adipoblasts, adipocytes, brown or white alpha cells, amacrine cells, beta cells, capsular cells, cementocytes, chief cells, chondroblasts, chondrocytes, chromaffin cells, chromophobic cells, corticotrophs, delta cells, Langerhans cells, follicular dendritic cells, enterochromaffin cells, ependymocytes, basal cells, squamous cells, transitional cells, erythroblasts, erythrocytes, fibroblasts, fibrocytes, follicular cells, germ cells, gametes, ovum, spermatozoon, oocytes, primary oocytes, secondary oocytes, spermatids, spermatocytes, primary spermatocytes, secondary spermatocytes, germinal epithelial cells, giant cells, glial cells, astroblasts, astrocytes, oligodendroblasts, oligodendrocytes, glioblasts, goblet cells, gonadotroph cells, granulosa cells, haemocytoblasts, hair cells, hepatoblast cells, hepatocytes, hyalocytes, interstitial cells, juxtaglomerular cells, keratocytes, lemmal cells, leukocytes, granulocytes, basophils, eosinophils, neutrophils, lymphoblasts, B-lymphoblasts, T-lymphoblasts, lymphocytes, B-lymphocytes, T-lymphocytes, helper induced T-lymphocytes, Th1 T-lymphocytes, Th2 T-lymphocytes, natural killer cells, thymocytes, macrophages, Kupffer cells, alveolar macrophages, foam cells, histiocytes, luteal cells, lymphocytic stem cells, lymphoid cells, lymphoid stem cells, macroglial cells, mammotroph cells, mast cells, medulloblasts, megakaryoblasts, megakaryocytes, melanoblasts, melanocytes, mesangial cells, mesothelial cells, metamyelocytes, monoblasts, monocytes, mucous neck cells, muscle cells, cardiac muscle cells, skeletal muscle cells, smooth muscle cells, myelocytes, myeloid cells, myeloid stem cells, myoblasts, myoepithelial cells, myofibrobasts, neuroblasts, neuroepithelial cells, neurons, odontoblasts, osteoblasts, osteoclasts, osteocytes, oxyntic cells, parafollicular cells, paraluteal cells, peptic cells, pericytes, peripheral blood mononuclear cells, phaeochromocytes, phalangeal cells, pinealocytes, pituicytes, plasma cells, podocytes, proerythroblasts, promonocytes, promyeloblasts, promyelocytes, pronormoblasts, reticulocytes, retinal pigment epithelial cells, retinoblasts, small cells, somatotrophs, stem cells, sustentacular cells, teloglials cell, or zymogenic cells, or the like.

In some embodiments, the eukaryotic cell is a neoplastic cell. In some embodiments, the target cell can be a transformed cell. In some embodiments, the eukaryotic cell is a virally infected cell, e.g., a cell that is infected with HIV, HCV, HBV or the like. In some embodiments, the eukaryotic cell can be a bacterially infected cells, e.g., a cell harboring mycoplasma, or the like. In some embodiments, the target cell can be a cell that expresses, or overexpresses, a particular cell surface marker. For example, in some embodiments, the target cell can express a tumor antigen such as prostate specific antigen, beta 2 micorglobulin, CA125 ovarian cancer antigen, CA 19-9, chromogranin A, thyroglobulin, CA 15-3, TA 90 and the like.

In some embodiments, the cells are contacted with a concentration of the compositions disclosed herein between about 0.001 μM and about 100 μM, e.g., between about 0.01 μM and about 10 μM, between about 0.5 μM and about 3 μM of the complexes described herein. In some embodiments, the cells can be contacted with a concentration of the compositions disclosed herein between about 1 pM and about 1 mM.

### Methods of Treating Subjects

In another aspect, the embodiments relate to methods of treating a subject with the compositions disclosed herein. The terms “subject,” “patient” or “individual” as used herein refer to a vertebrate, preferably a mammal, more preferably a human. “Mammal” can refer to any animal classified as a mammal, including humans, domestic and farm animals, and zoo, sport, or pet animals, such as, for example, horses, sheep, cows, pigs, dogs, cats, etc. Preferably, the mammal is human.

As used herein, the terms “treatment,” “treating,” and the like, refer to obtaining a desired pharmacologic and/or physiologic effect. The effect may be prophylactic in terms of completely or partially preventing a disease or condition or symptom thereof and/or may be therapeutic in terms of a partial or complete cure for a disease or condition and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it and/or has been diagnosed but is not yet symptomatic; (b) inhibiting the disease, i.e., arresting its development; and (c) relieving the disease, i.e., causing regression of the disease.

The subject can be administered a therapeutically effective amount of a composition disclosed herein. A “therapeutically effective amount” as used herein includes within its meaning a non-toxic but sufficient amount of a compound or composition for use in the invention to provide the desired therapeutic effect. The exact amount of the active ingredient disclosed herein required will vary from subject to subject depending on factors such as the species being treated, the age and general condition of the subject, co-morbidities, the severity of the condition being treated, the mass of the subject, the particular agent being administered and the mode of administration and so forth. Thus, it is not possible to specify an exact “effective amount”. However, for any given case, an appropriate “effective amount” may be determined by one of ordinary skill in the art using only routine methods. Optimum dosages of the compositions disclosed herein can vary depending on the relative potency of individual nucleic acids (e.g., RNA interfering agents), and can generally be estimated based on EC₅₀s found to be effective in in vitro and in vivo animal models. In general, dosage is from 0.01 μg to 1 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the biologically active nucleic acid delivery complex is administered in maintenance doses, ranging from 0.01 μg to 1 g per kg of body weight, once or more daily, to once every 20 years.

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates.

Having now generally described the invention, the same will become better understood by reference to certain specific examples which are included herein for purposes of illustration only and are not intended to be limiting unless otherwise specified. All referenced publications and patents are incorporated, in their entirety by reference herein.

### EXAMPLES

### Example 1

### siRNA:RND-DRBD Complexes Form Insoluble, Active Complexes

The experiments described in this example demonstrate the unexpected finding that large multimeric complexes of siRNA and a DRBD-PTD chimera are significantly more active than smaller complexes of siRNA and a DRBD-PTD chimera.

Briefly, 50 μM stock of DRBD-PTD chimera prepared in PBS pH 7.0 was mixed with a 5 μM stock of GADPH (NM_{—}002046) siRNA (Ambion, AM16099) or control siRNA (Ambion, AM4636 #1) prepared in nuclease-free water to yield mixtures having a final DRBD-PTD chimera concentration of 3.2 μM or 1.8 μM, and a concentration of siRNA of 0.1, 0.03, 0.01, 0.003, or 0.001 μM. The DRBD-PTD chimera and siRNA were mixed by gentle vortexing, and incubated for 30 minutes at room temperature, at which point the mixture appeared cloudy. A duplicate set of reactions were performed. In the first set of reactions, the cloudy mixture was used in the transfection experiments. The second set of reactions were centrifuged at 13K rpm for 15 min, to clarify the cloudy mixture following the 30 minute incubation and the clear supernatant layer was carefully removed and used in the transfection experiments.

The two sets of mixtures described above were subsequently used to transfect human primary chondrocyte cells. For transfection, the mixtures were diluted with serum-free media (“SFM”) and added to 6×10³ cells of human primary chondrocytes in 96-well plate. The cells were incubated for 3 hours at 37° C./5% CO₂, at which time point the transfection mixtures were removed and the cells were washed with tissue culture media containing 10% fetal bovine serum (FBS) and 100 uL of this media was then added to each well. The cells were incubated at 37° C./5% CO₂ for 24 hours.

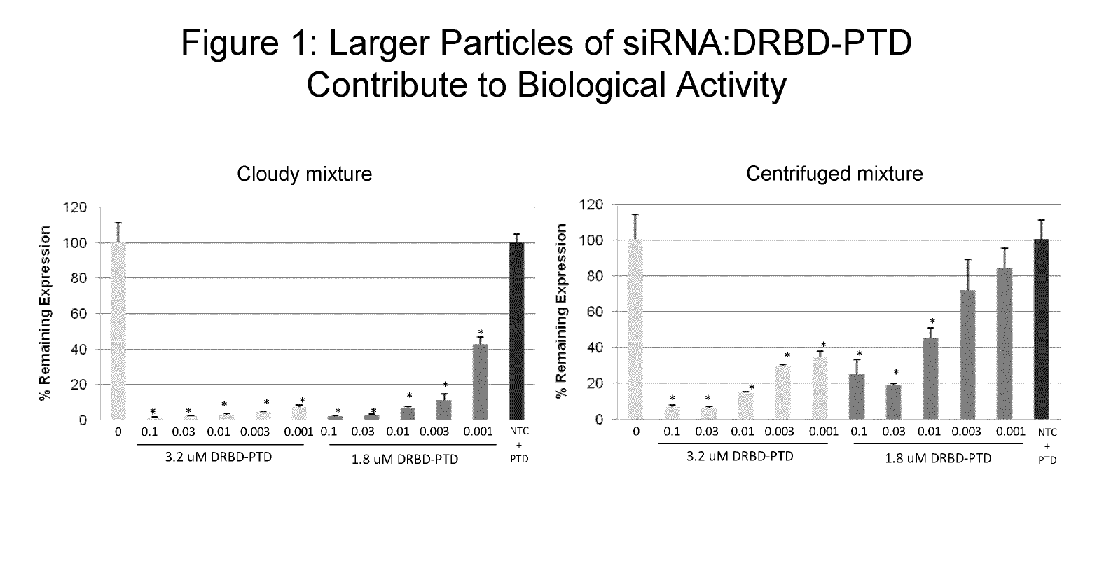

Cells were then lysed and total RNA was isolated from the cells using Qiagen RNeasy kit according to manufacturer's instructions. cDNA was made using 25 ng total RNA and RT-qPCR was performed on 10 ng cDNA using GAPDH sequence-specific primers and 6FAM-dye-labeled probe. The amount of amplification product was quantified, and the level of GADPH transcript was normalized to beta-2 macroglobulin (a house-keeping gene). The amount of normalized GAPDH transcript from the untreated wells was set to 100%. The data are presented in FIG. 1. A comparison of the % remaining GADPH expression between cells that were transfected with the cloudy (uncentrifuged) mixture or the centrifuged mixture reveals that the cloudy (uncentrifuged) mixture shows that for each of the concentrations of siRNA used, the uncentrifuged mixture exhibited significantly more efficient transfection efficiency compared to the corresponding centrifuged mixture. This trend was observed in the experiments in which the DRBD-PTD chimera was present at 3.2 μM or 1.8 μM.

The observation that the activity of the complexes appeared to reside within the cloudy mixture, which, as discussed in further detail below includes aggregates that form large particles, versus the clear solution, was unexpected.

The following Example describes experiments that demonstrate that the observed loss of activity of the siRNA:DRBD-PTD complexes upon centrifugation is also observed with the passage of time.

### Example 2

### DRBD-PTD Chimeras and siRNA Form Thermodynamically Stable, Low-Energy Complexes that Exhibit Decreased Activity

Applicants observed that the cloudiness observed upon mixing DRBD-PTD chimeras with siRNA disappeared over time. To determine whether the activity of the siRNA:DRBD-PTD complexes decreased over time as the cloudiness of the mixtures disappeared, a time course was performed to assess the activity of various siRNA:DRBD-PTD complexes over time. Briefly, 44, 11, 2, or 0.4 μM of DRBD-PTD were combined with 44, 11, 2, or 0.4 μM GADPH siRNA, respectively, in PBS pH 7.0. The DRBD-PTD chimera and siRNA were mixed by vortexing, and allowed to incubate for 30 minutes, 4 hours, or 24 hours at room temperature. By the 24 hour time point the mixtures all had a clear appearance.

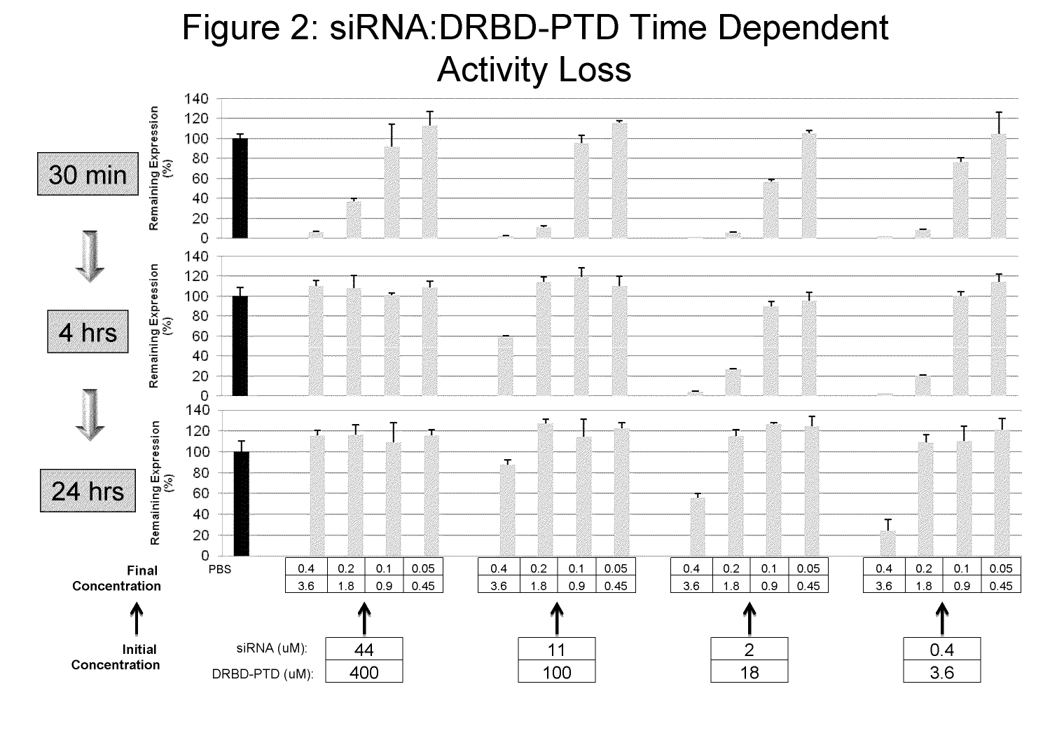

For transfection, the mixtures were diluted in SFM to the final concentrations indicated in FIG. 2 and added to 6×10³ human primary chondrocyte cells as described in Example 1.

The cells were lysed and total RNA was isolated from the cells using Qiagen RNeasy kit according to manufacturer's instructions. cDNA was made using 25 ng total RNA and RT-qPCR was performed on 10 ng cDNA using GAPDH sequence-specific primers and 6FAM-dye-labeled probe. The amount of amplification product was quantified, and the level of GADPH transcript was normalized to beta-2 macroglobulin house-keeping gene. The amount of normalized GAPDH transcript from the untreated wells was set to 100%. The data are presented in FIG. 2. The transfection efficiency for each of the tested concentrations decreased significantly over time, consistent with the observation that active siRNA:DRBD-PTD delivery complexes are not stable over time, and lose activity. These data also suggest that the PTD of the DRBD-PTD loses its protein transduction functionality over time as it begins to bind to the dsRNA, e.g. it does not function as a PTD, but instead functions to neutralize the charge of the dsRNA by binding to the dsRNA, i.e., as an RNA neutralization domain. The interaction between the DRBD-PTD (now recognized as an RND-DRBD) and the dsRNA appeared to change, reaching equilibrium over time, as evidenced by the appearance and size of particles present in the mixture as more stable complexes are formed between the proteins and RNA. These data explain the greater activity observed for the complexes from the cloudy mixture described in Example 1; when the DRBD-PTD is initially mixed with the dsRNA, large aggregated particles form which have a significant amount of exposed PTD, allowing for cell surface interaction, transport across the cell membrane and RNA interference. Over time, these particles rearrange to a more thermodynamically stable state whereby the positively charged PTD molecules bind to the negatively charged dsRNA molecules, thereby becoming more soluble (clear solution), although biologically inactive, as the PTD molecules are no longer available to act as a PTD. Rather, the domain is not functioning as a PTD, but is instead functioning as an RND.

The data in FIG. 2 confirm the unexpected results from the experiments in Example 1/FIG. 1, namely that the activity of the siRNA:DRBD-PTD complexes resides in the “cloudy” mixture, and that the biologically active complexes of siRNA: DRBD-PTD are neither stable nor active over time.

The following example describes experiments that demonstrate that the ratio of siRNA:DRBD-PTD chimera has an effect on the activity of the complexes, and that the PTD portion of the DRBD-PTD chimera functions as an RNA neutralization domain (RND), rather than a protein transduction domain.

### Example 3

### The Ratio of siRNA:DRBD-PTD Chimera Impacts the Activity of the Complex; and the PTD Domain Functions as an RNA Neutralization Domain

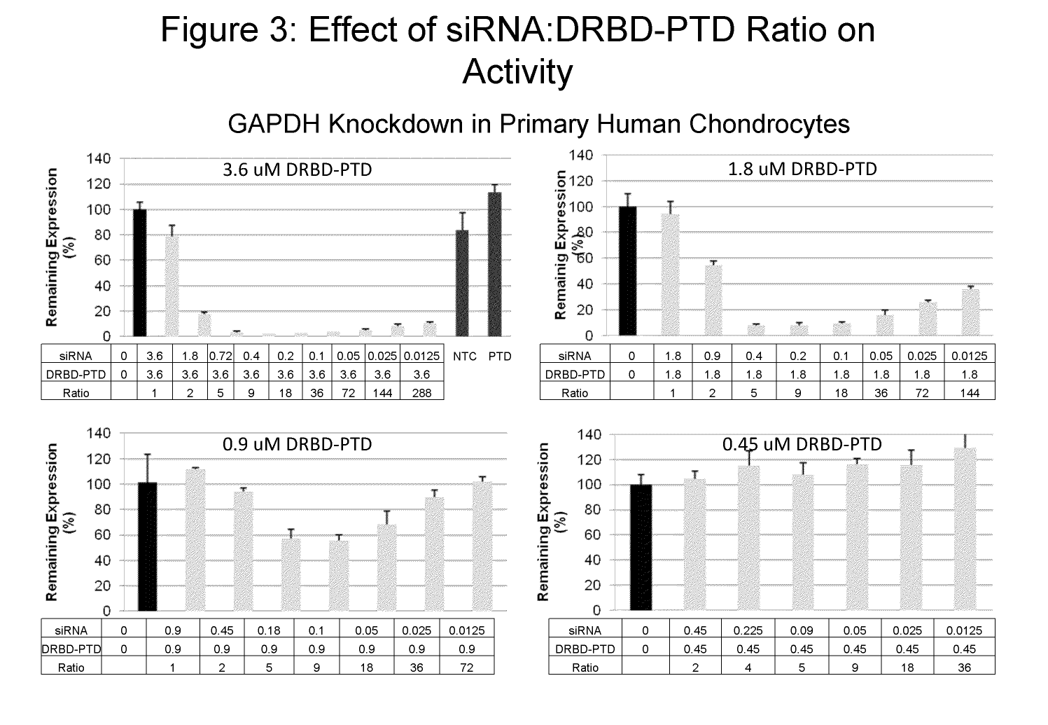

In order to determine whether the ratio of siRNA:DRBD-PTD chimera has an effect on the activity of the complexes, complexes were formed at various ratios of siRNA:DRBD-PTD chimeras. Briefly, GADPH (NM_{—}002046) siRNA (Ambion, AM16099) was added to either 3.6 μM DRBD-PTD or 1.8 μM DRBD-PTD to final siRNA concentrations as indicated in FIG. 3. These mixtures were incubated for 30 min at room temperature and used to transfect human chondrocytes as described in Example 1.

GADPH expression in each of the reactions was determined using the method described in Example 1, and expressed as % remaining expression. The data are presented in FIG. 3.

Complexes formed with a 1:1 ratio of siRNA:DRBD-PTD chimera exhibited no significant activity, as compared to the controls. Active complexes were observed in complexes formed with a 1:2 ratio of siRNA:DRBD-PTD chimera, or greater, suggesting that some type of transition occurs when the siRNA is contacted with 2 molar equivalents of DRBD-PTD chimera.

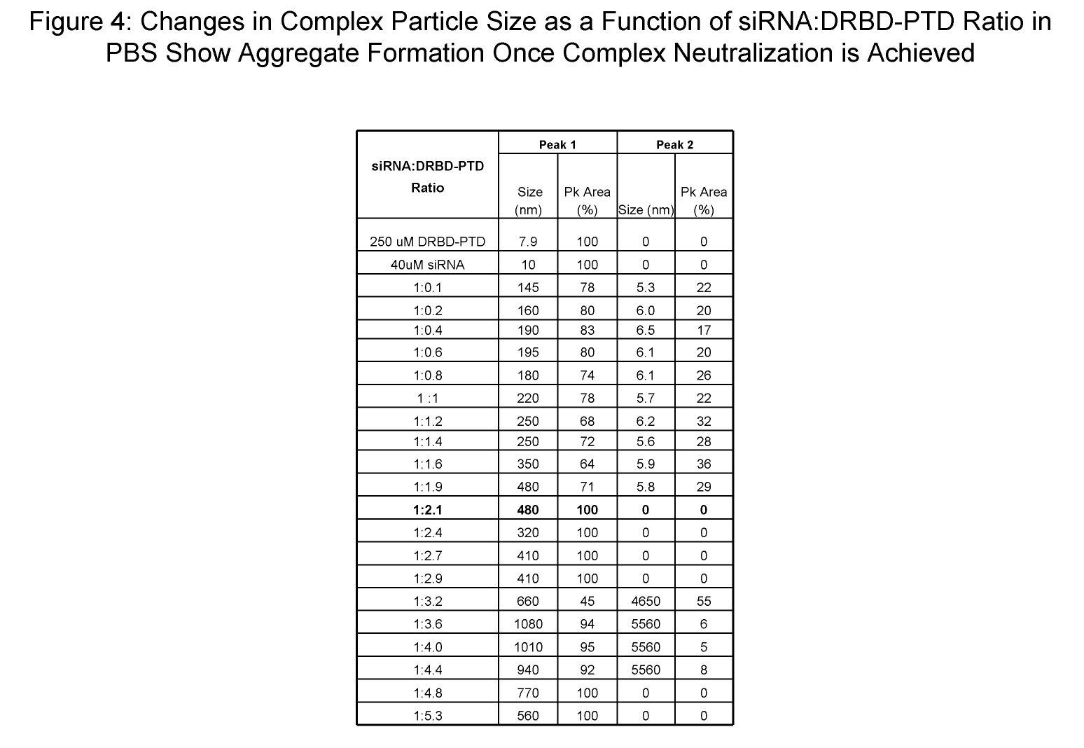

In order to explore the observed transition further, dynamic light scattering was used to calculate the particle size of siRNA:DRBD-PTD chimera complexes formed using various ratios of siRNA:chimera, ranging from 1:0.1 to 1:5.3, as indicated in FIG. 4. A 250 μM stock solution of DRBD-PTD chimera and a 40 μM stock solution of GADPH siRNA, both in PBS pH 7.0 were used in the experiments. The DRBD-PTD chimera and siRNA were mixed and allowed to incubate for 30 min. 100 μL of the solution was analyzed in a ZEN6300® dynamic light scattering machine (Malvern Zetasizer). The data regarding the particle sizes present in the various mixtures of siRNA:DRBD-PTD are presented in FIG. 4. At a ratio of 1:2 siRNA:DRBD-PTD chimera, the smaller (2^{nd} peak) particles that were present in the lower ratios of siRNA:DRBD-PTD disappear, and 100% of the observed complexes had a size of about 480 nm. Between the 1:2.9 and 1:3.2 ratios, much larger particles appeared. At higher ratios of siRNA:DRBD-PTD (1:3.2), the size of the larger particles increases dramatically. The data suggest that at 2 equivalents of chimeric protein to siRNA, the siRNA:DRBD-PTD reaches a binding equilibrium and the siRNA binding becomes saturated, and that below this ratio, excess DRBD-PTD that is not in complex with the dsRNA forms aggregates.

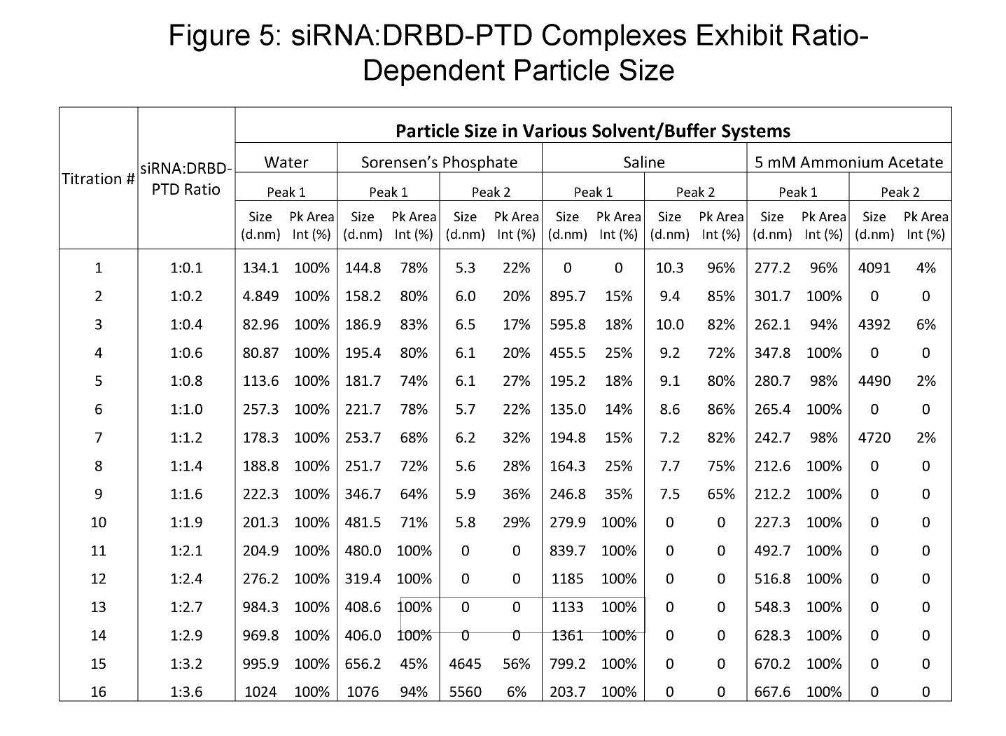

Similar sets of experiments were performed in water, Sorensen's phosphate buffer, normal saline and 5 mM ammonium acetate. The data are presented in FIG. 5. In all cases, upon the initial additions of DRBD-PTD chimera (up to about a 1:1 ratio), the size of the complexes increases to approximately 200 nm. In cases where small particles exist initially (e.g. Sorensen's buffer and saline), these particles disappear at ratios near 1:2 (siRNA:DRBD-PTD). In all cases, at ratios above 1:2 to 1:3, a significant increase in particle size is observed as large, insoluble particles begin to form after the siRNA becomes neutralized. Similar effects were observed in other buffered systems. For particles at the 1:2 ratio, the size was observed to decrease over time in both water and PBS pH 7.0 (Data not shown).

The data indicate that upon mixing in a variety of aqueous based solvents, the particles are initially large and in a metastable state, wherein early interactions between the DRBD-PTD chimera and siRNA are dominated by strong ionic bond formation between the negatively charged phosphate groups on the siRNA backbone and the positively charged groups of the arginine side chains of the PTD domain, likely forming a crosslinked network of large particles. Over time, a thermodynamically driven rearrangement occurs, wherein both the PTD domain and the DRBD that is tethered to the PTD domain form strong interactions with the siRNA, thereby completing the neutralization of the nucleic acid, resulting in the formation of smaller particles as crosslinks are broken and the system approaches a more stable state.

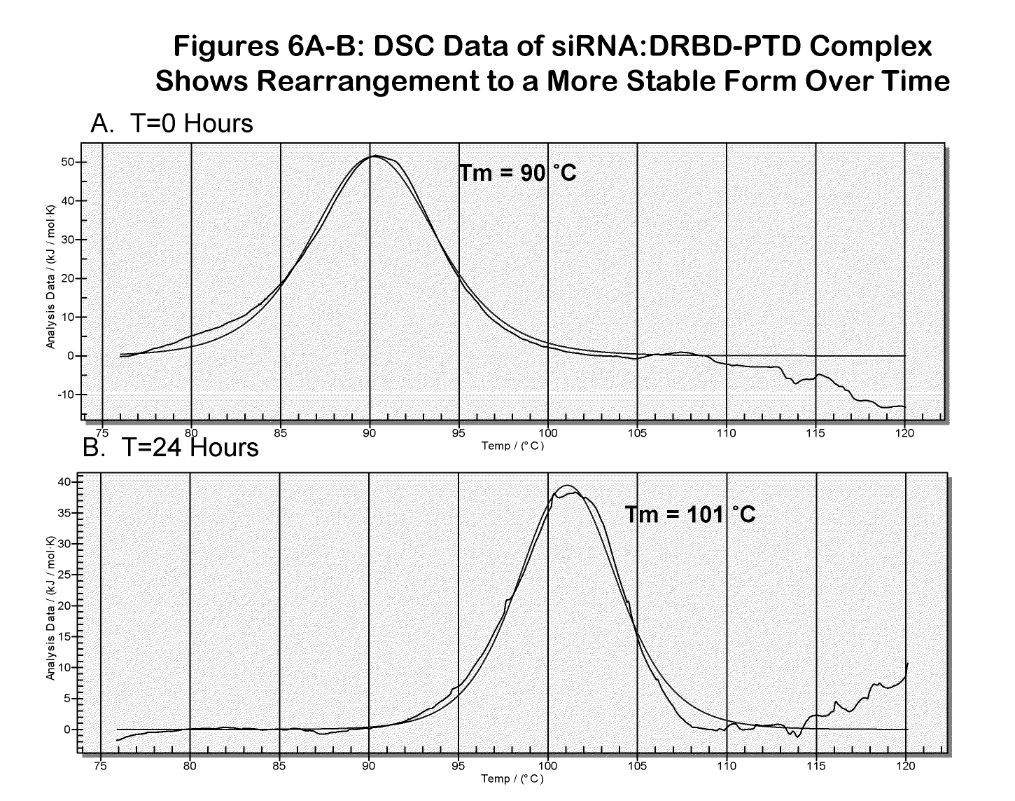

In order to further confirm that the siRNA and the DRBD-PTD chimeras were forming thermodynamically stable complexes over time, the kinetics of dissociation of the complexes was assessed using differential scanning calorimetry. FIG. 6 shows differential scanning calorimetry data (DSC) on two identical siRNA:DRBD-PTD complexes, one where the siRNA and DRBD-PTD are mixed and scanned immediately, the second where the components were mixed and then scanned after allowing the sample to equilibrate for 24 hours. The transition temperature (Tₘ) measured at approximately 90° C. for the T=0 sample is indicative of the unwinding of the strands of the siRNA partially bound to the RND-DRBD. However, for the 24 hour sample, the transition temperature for this event is greater than 100° C. The increase in the transition temperature is likely due to the greater number of RND-DRBD interactions with the siRNA occurring over time, necessitating more energy input to break them, thus the higher Tₘ. These data are in agreement with the particle size data shown above, and indicate that partial binding of the siRNA to the DRBD occurs initially after mixing, and over time a thermodynamically driven rearrangement occurs, wherein both the PTD domain and the DRBD that is tethered to the PTD domain form more bonds and thus stronger interactions with the siRNA.

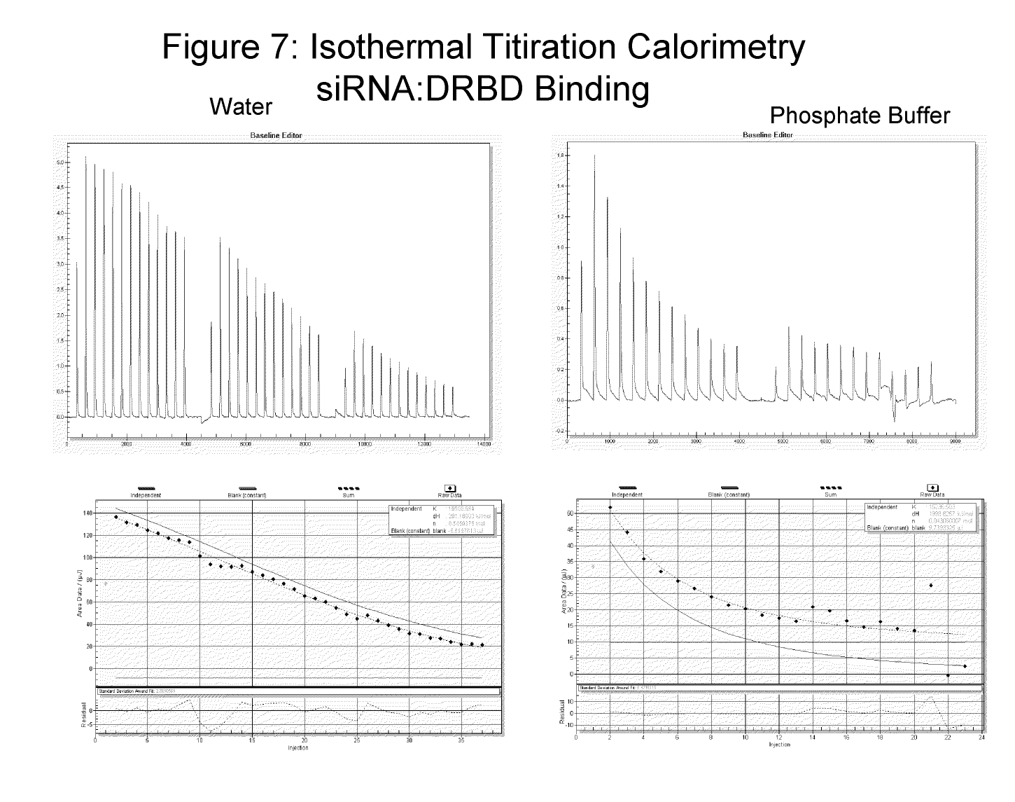

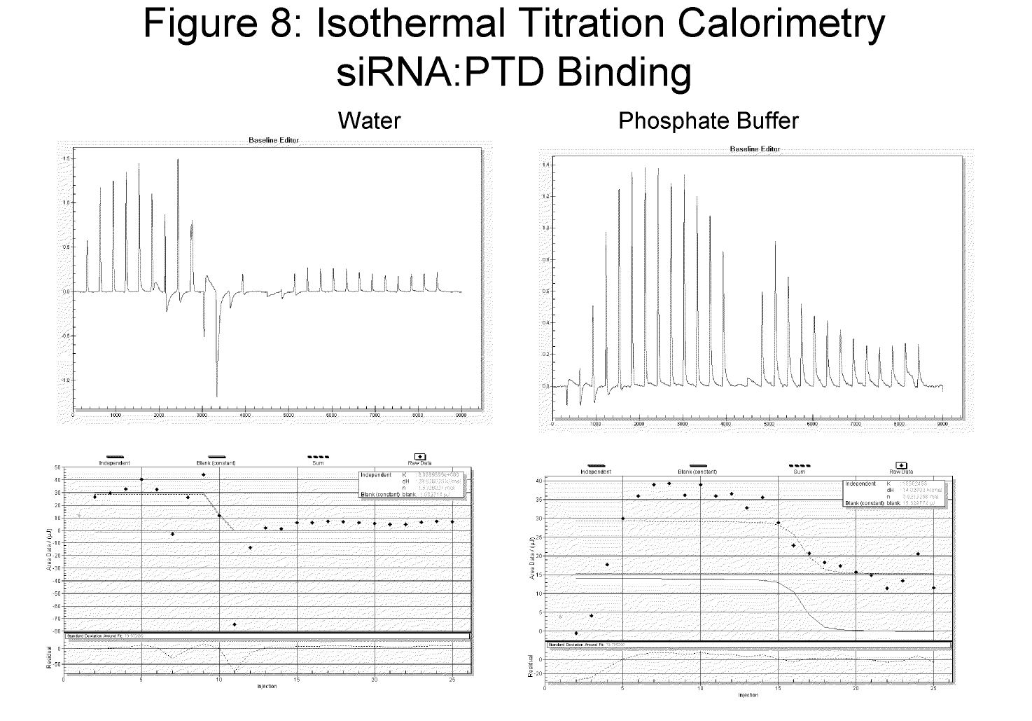

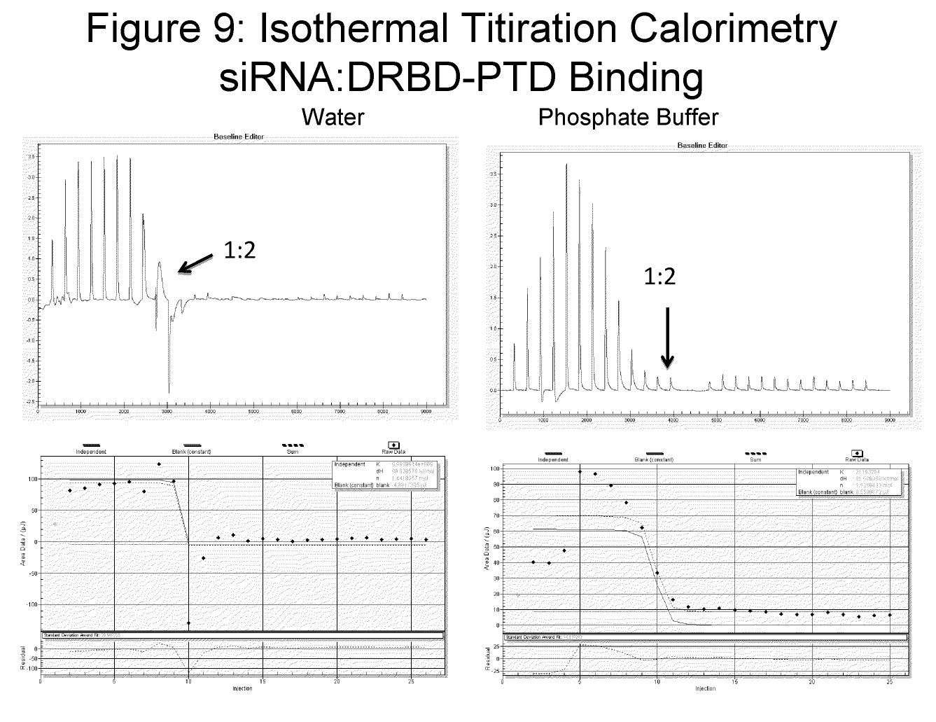

To confirm this finding, isothermal titration calorimetry (ITC) was used to examine the affinity of the DRBD, RND, and RND-DRBD chimera for the dsRNA, as well as the stoichiometry and thermodynamics of the interactions. The data are presented in FIGS. 7-9. The DRBD exhibited a very weak Kₐ for the dsRNA, both in water and in buffer, indicative of slow and/or weak initial binding. (FIG. 7). The AH of the interaction was much higher when the solvent was buffered (e.g., phosphate), as compared to when a pure aqueous system was used as the solvent. In both cases, the data indicate strong binding, but very slow kinetics. (FIG. 7). By contrast, the Kₐ of the PTD domain and dsRNA was much higher, in both water and in PBS (although the Kₐ was much higher in the pure aqueous system as compared to the phosphate buffered system), compared to the DRBD domain. However, for the PTD domain (in both solvent systems) the AH was substantially lower than observed for the binding of DRBD alone. PTD and dsRNA exhibited a low initial binding heat and an exothermic response when saturation binding was reached. (FIG. 8). The DRBD-PTD chimera exhibited a Kₐ consistent with the PTD domain alone, indicating that the PTD domain of the chimera binds to the dsRNA rapidly (albeit less tightly initially than the DRBD as evidenced by the lower AH values in both cases), and with a high affinity, and the strong dsRNA:DRBD binding may operate through an avidity/proximity effect, in the context of the DRBD-PTD chimera. (FIG. 9). The lower overall Kₐ values for each of the PTD, DRBD and DRBD-PTD chimera in the presence of buffer can be explained by the presence of the counterions from the buffer acting to reduce the charge based affinity between the components and the siRNA. Once a critical mass of PTD, DRBD or DRBD-PTD is present (generally 0.5 equivalents), enough mass is present to displace the counterions allowing for more complete binding to occur. This is can be seen by the complete and rapid binding observed in cases where charge based binding is the dominant mechanism (PTD and DRBD-PTD) e.g. as evidenced by the binding data shown in the pure aqueous systems, where there are few if any counterions in the solvent system.

The data confirm the observations in Example 1, namely, that the PTD domain in the DRBD-PTD chimera functions as an RND neutralization domain (now recognized as an RND-DRBD chimera), that forms rapid, strong, ionic charged based interactions with dsRNA. This discovery explains the loss of activity of the siRNA:RND-DRBD complexes observed over time, as the charged residues of the RND domain of the RND-DRBD chimera are forming bonds with the dsRNA, thereby neutralizing the dsRNA. When the charged residues of the PTD function as RNA neutralization moieties, the PTD domain becomes inactive as a PTD, as there are no longer any positively charged groups available for interaction with a cell surface to stimulate uptake. This explains the loss of activity of the siRNA:RND-DRBD complexes over time, in that initially (before complete RND and/or DRBD binding to the siRNA) there is still some unbound RND which can interact and function as a PTD. After the complex reaches a thermodynamically stable state and the RND has bound completely to the siRNA, there is no further potential for cell surface interaction and activity of the complex is lost.

The isothermal titration calorimetry data is summarized in Table 2, below:

250 uM Protein into 40 uM Luciferase siRNA

| | | Equilibrium |
|---|---|---|
| | Enthalpy (ΔH) | Constant |
| Sample Type | (kJ · mol−1) | (K) |
| DRBD | 201.18 | 1.05E+04 |
| in | | |
| DI Water | | |
| DRBD | 1998.83 | 1.52E+04 |
| in | | |
| Sorensen's | | |
| Buffer | | |
| PTD | 29.83 | 1.00E+10 |
| in | | |
| DI Water | | |
| PTD | 14.03 | 1.04E+07 |
| in | | |
| Sorensen's | | |
| Buffer | | |
| DRBD-PTD | 98.92 | 1.00E+10 |
| in | | |
| DI Water | | |
| DRBD-PTD | 61.51 | 2.12E+07 |
| in | | |
| Sorensen's | | |
| Buffer | | |

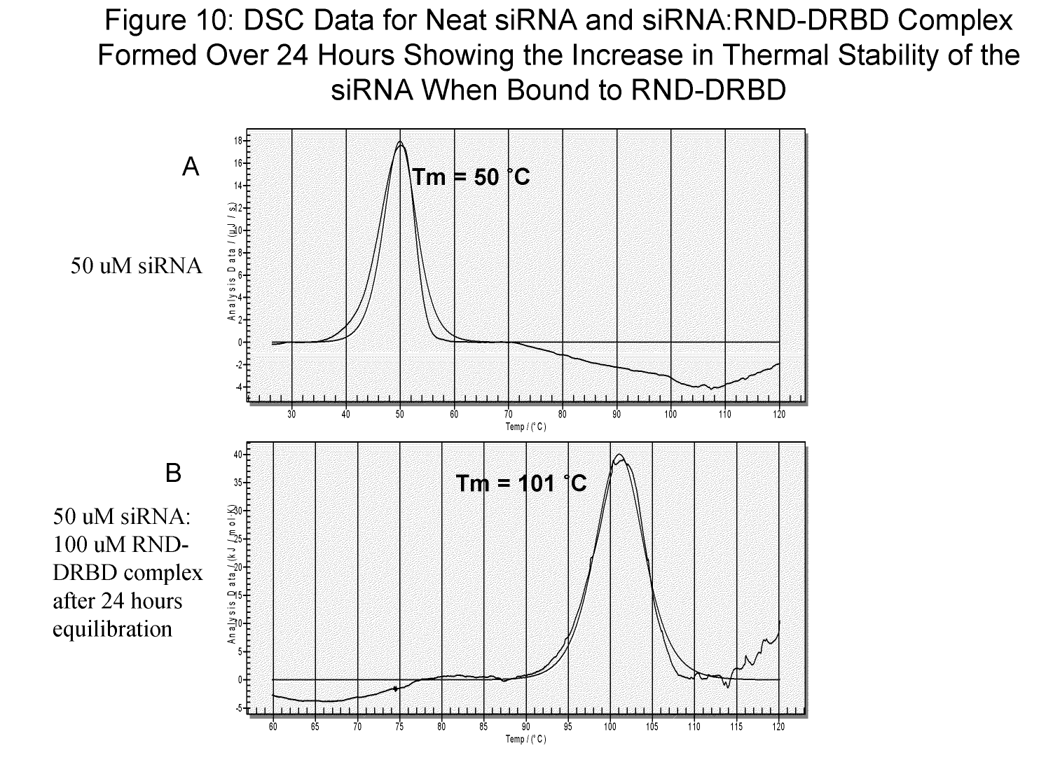

In order to further confirm the stability of the siRNA:RND-DRBD complexes, differential scanning calorimetry was performed on pure siRNA (neat siRNA) and on siRNA equilibrated for 24 hours with RND-DRBD chimera. FIG. 10 shows the data for the DSC scans. The RND-DRBD exhibited a stabilizing effect on the siRNA, as exhibited by a dramatic increase in the Tₘ of the siRNA when in the equilibrated siRNA:RND-DRBD complex. Specifically, the Tₘ for the siRNA is 50° C. while the Tₘ for the siRNA:RND-DRBD complex is 101° C. When the RND-DRBD is given enough time to reach thermodynamic binding equilibrium with the siRNA in the mixture, significant binding interactions occur and do so to the extent that the siRNA resists strand separation/unwinding until a significantly greater amount of energy input is provided. The difference in Tₘ values is substantial (−50° C.) and further indicates the extent of binding between the siRNA and the RND-DRBD of the thermodynamically stable form of the complex.

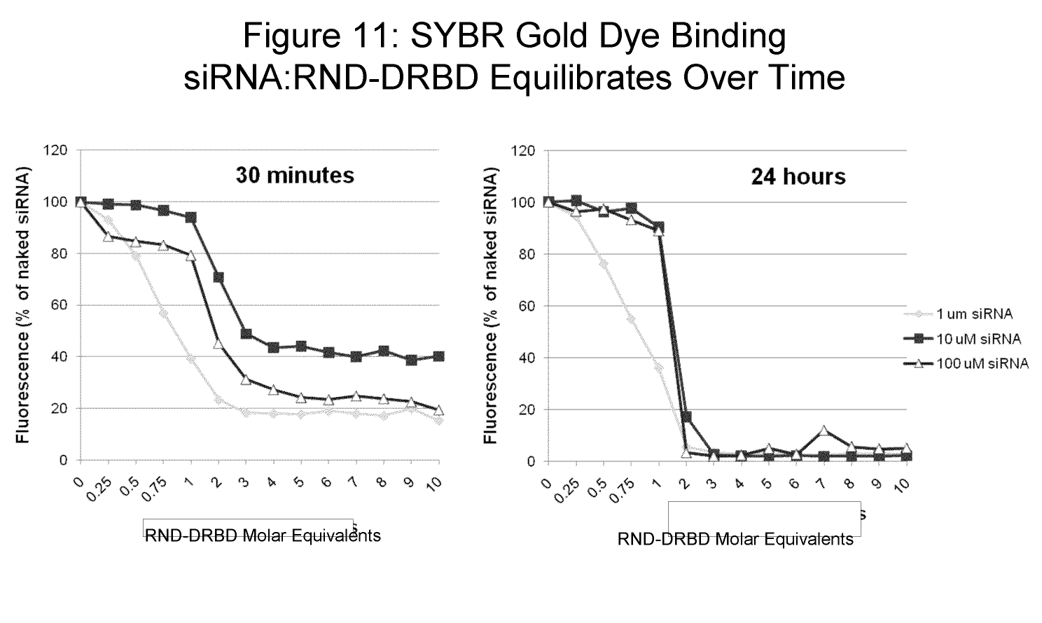

To further confirm that the PTD domain is functioning as an RNA neutralization domain (RND) in the chimeras, SYBR Gold dye binding experiments were used to measure the available siRNA binding sites in the presence of different molar equivalents of chimera. This dye only fluoresces when bound to the bases pairs of a nucleic acid. Briefly, various molar equivalents (shown in FIG. 11) of RND-DRBD chimera were mixed with three different concentrations (1 μM, 10 μM or 100 μM) of siRNA. The mixtures were allowed to incubate for 30 minutes or for 24 hours at room temperature, at which time the SYBR Gold dye was added to the mixture and the fluorescence measured. The data in FIG. 11 demonstrate that as more equivalents of RND-DRBD are added, the available binding sites of the siRNA start to decrease. In all cases, siRNA binding saturation is observed at a stoichiometry between 1:2 and 1:3 (siRNA:RND-DRBD). For the 30 minute sample, although saturation at the indicated stoichiometry is demonstrated, significant background fluorescence is still observed. This indicates that while there is binding occurring initially, this binding is incomplete, allowing the SYBR Gold dye some access to the siRNA. Conversely, for the samples where the siRNA and RND-DRBD were allowed to equilibrate for 24 hours, saturation occurs at the same stoichiometry but no background fluorescence is observed. The comparison of the fluorescence level of the mixtures at 30 minutes to 24 hours confirm that the RND-DRBD structurally re-arranges around the siRNA over time and the siRNA becomes completely masked by the RND-DRBD chimera, such that no siRNA binding sites are available. Unlike the isothermal titration calorimetry experiments described above, the 24 hour data from this experiment represent the system in a state much closer to thermodynamic equilibrium. The difference in background fluorescence between 30 minutes and 24 hours is indicative of the re-arrangement of the RND-DRBD over time, allowing for more complete coverage of the siRNA, likely due to the kinetically slower, but stronger binding of the DRBD and/or completion of the initial binding by the RND. These data further illustrate the cooperative effect of binding between the RND and the DRBD to the siRNA, and confirm the observations from the experiments described above.

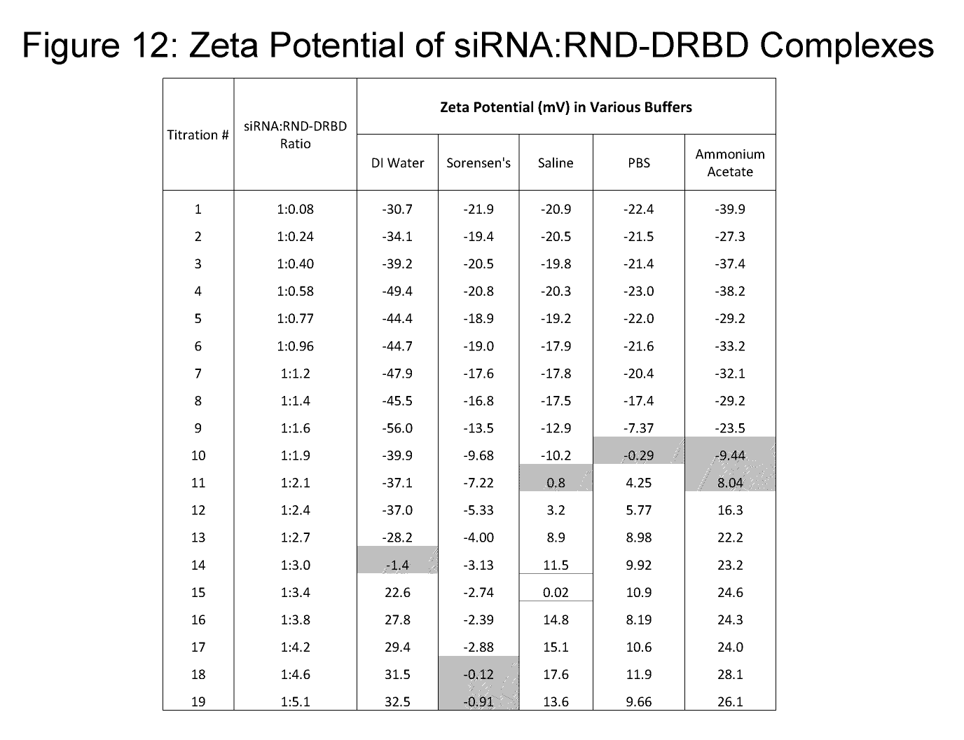

The zeta potential of dsRNA:RND-DRBD chimera complexes formed with varying ratios of RND-DRBD to siRNA was measured in various solvent/buffer systems as another way to confirm that the RND-DRBD chimera functions to bind and mask the negative charge of the dsRNA. The type and quantity of buffer species present in the mixture can have an impact on the absolute values of the zeta potential of the mixture. The data are presented in FIG. 12. Solutions of pure, non complexed siRNA have substantially negative zeta potential values which become increasingly more positive as RND-DRBD is added and binds to the siRNA. For most buffer systems, a near neutral zeta potential is observed at ratios between 1:1.9 and 1:3. For a Sorensen's buffer system, a neutral zeta potential is observed at higher levels of RND-DRBD (˜1:5), most likely due the much higher concentration of counterions in this buffer, requiring larger amounts of RND-DRBD to displace them in order to bind the siRNA. These data are consistent with the RND-DRBD binding to and masking the charge on the dsRNA.

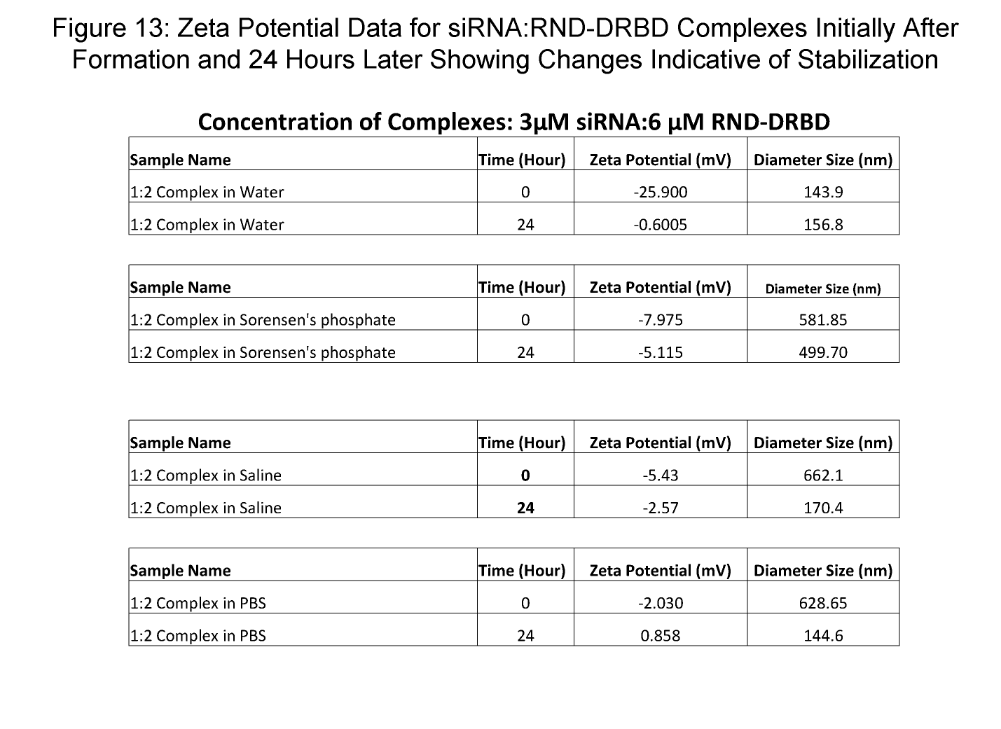

In order to confirm that the RND-DRBD chimera functions to mask the charge on the dsRNA, zeta potential measurements were taken immediately following mixture of siRNA with RND-DRBD chimeras at a ratio of siRNA:RND-DRBD of 1:2, and 24 hours later on the same sample. The data are shown in FIG. 13. For the pure aqueous system, the initial zeta potential value was significantly negative, likely due to incomplete RND-DRBD binding at this stage of mixing. After 24 hours, the zeta potential value was nearly neutral, indicating additional binding and/or rearrangement of the RND-DRBD and siRNA had occurred, reducing the negative charge on the siRNA substantially. In this case, the particle size of the neutralized complexes did not change substantially from initial values. For all other buffer systems, similar changes in zeta potential are observed over time (becoming more positive and closer to neutrality). Furthermore, decreases in the particle size are also observed over time, indicating that large aggregates of initially unstable, crosslinked particles and/or networks of siRNA and RND-DRBD are breaking down into more stable, neutralized complexes. These data show the stabilization of the complex and the rearrangement that occurs to a more thermodynamically stable state over time after initial binding.

Together the data from this example and Examples 1-3 demonstrate RND-DRBD binds to dsRNA rapidly but incompletely initially. Over time completes its binding to dsRNA and upon reaching an equilibrium binding state, there is no charge available on the RND (as it is complete bound to the oppositely charged functional groups of the dsRNA) to interact with cell surfaces rendering it ineffective as a PTD.

These data suggest that the covalent incorporation of one or more PTDs to the stable, low energy complexes (wherein the charge state of the dsRNA is substantially or completely neutralized) described herein (to either the RND and/or DRBD) could facilitate delivery of the complex across cell membranes, since the propensity for the PTD to bind to the dsRNA through charge-charge interactions would be nearly or completely ablated. This would allow the added PTD to maintain its positive charge and allow it to function as a true PTD, enabling and/or facilitating cell surface interactions and transport across the cell membrane.

### Example 5

### PTDs do not Bind Thermodynamically Stable Complexes of siRNA:RND-DRBD

PTDs, when present in the compositions disclosed herein, are characterized in part as having substantially no strong ionic charge-based interaction or association with the stable nucleic acid delivery systems of the compositions disclosed herein (e.g. dsRNA:RND-DRBD complexes), or at least substantially less strong, ionic charge-based interactions with the nucleic acid delivery systems of the compositions herein. In order to confirm that PTDs do not interact with or bind thermodynamically stable complexes of siRNA:RND-DRBD, isothermal titration calorimetry was used to determine the interaction of PTDs added to thermodynamically stable, equilibrated siRNA:RND-DRBD complexes.

Briefly, a stable 1:2 complex of siRNA:RND-DRBD was prepared. 30 μM GAPDH siRNA and 60 μM of RND-DRBD were each separately dialyzed into deionized water and then mixed to form a 1:2 complex. The complex was allowed to reach thermodynamic equilibrium over a 24 hour period. 120 μM PTD was titrated into the stable complex and the binding heat measured using an isothermal titration calorimeter (NanoITC, TA Instruments). FIG. 14 shows the binding heat data from the experiment (lower panel). No appreciable binding is observed, rather, the heat signals are erratic, both endothermic and exothermic, and are likely due to heat associated with mixing of dissimilar materials. The data could not be fit to a standard model for binding parameters estimation. Compared to the data shown in the upper panels (e.g., binding of PTD-DRBD to siRNA and binding of PTD to siRNA—each generated under similar experimental conditions), where no RND and/or DRBD has been previously complexed with the siRNA, it is clear that the RND-DRBD chimera substantially and/or completely neutralizes the charge on the siRNA, thus preventing further charge based interactions with subsequent additions of PTD.

### Example 6

### Delivery of Nucleic Acids within a siRNA:RND-DRBD Complex is Achieved by the Addition of PTDs

The following experiments demonstrate that the association of protein transduction domains with biologically inactive stable, low energy nucleic acid (e.g., RNA interfering agent):RND-DRBD complexes re-activates the complexes resulting in effective delivery of the nucleic acid into target cells.

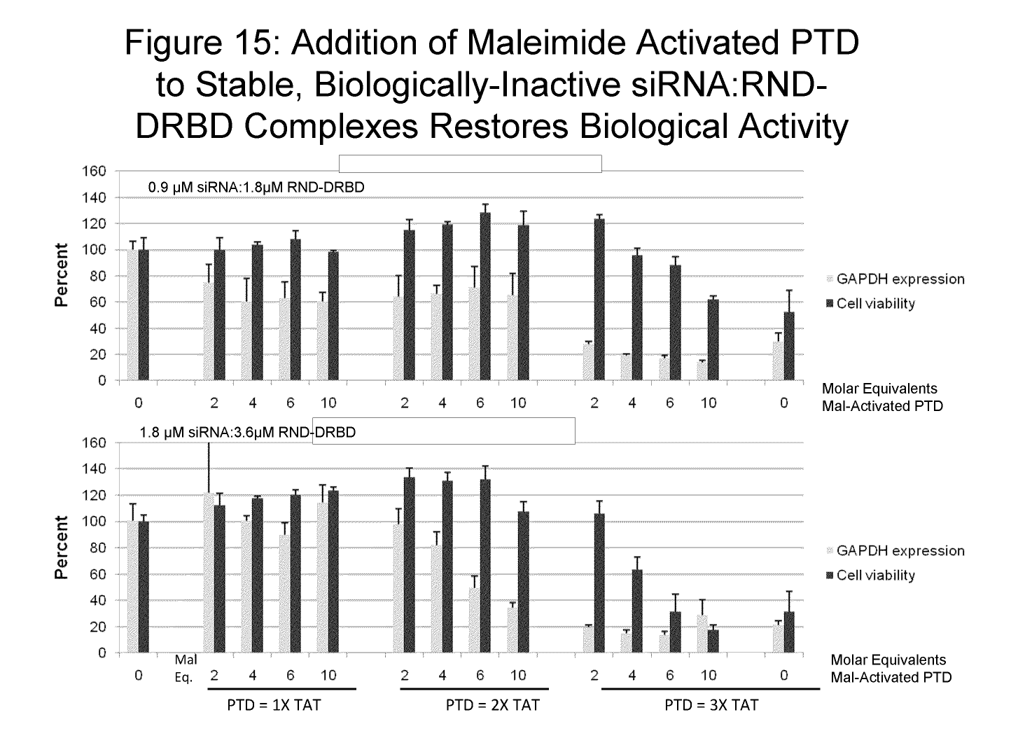

The experiments described in Examples 1-5 above demonstrate that stable, low-energy complexes are formed between dsRNA and RND-DRBD proteins, and that these stable, low energy complexes are biologically inactive. In order to determine whether the addition of one or more PTDs to the stable, but inactive complexes, could restore nucleic acid delivery, stable complexes of different DRBD-PTD complexes were formed by mixing dsRNA and DRBD-PTD chimeras at final concentrations of 0.9 μM siRNA:1.8 μM DRBD-PTD or of 1.8 μM siRNA:3.6 μM DRBD-PTD in PBS, pH 7.0. The complexes were incubated at room temperature for 24 hours, at which time the biological activity of these complexes is lost completely, as shown in FIG. 15 (negative control).

Maleimide-activated PTD reagents were prepared using conventional procedures. The maleimide reagent was coupled to a PTD peptide using an activated NHS ester under anhydrous conditions. The activated PTD domains were added to the dsRNA:RND-DRBD complexes at varying molar equivalents to the RND-DRBD chimeras (2, 4, 6, 8, and 10 molar equivalents of maleimide-activated PTD). The reaction was incubated at room temperature for 3 hours, in order to conjugate the maleimide-activated PTD to the RND-DRBD chimera already bound stably to the siRNA.

RND-DRBD protein was dialyzed into 0.9% physiological saline (pH 7) overnight at 4° C. The dialysate was filtered through a 0.2 micron filter. A GADPH/Luciferase siRNA was dissolved in nuclease free water and maleimide-activated PTD reagents were dissolved in PBS buffer (pH 7). The appropriate volume of protein was added to the siRNA, vortexed gently and incubated at room temperature for 24 hours to create a 1:2 complex of dsRNA:RND-DRBD. The maleimide-activated PTD reagent was then added to the stabilized 1:2 complex and incubated for 3 hours to conjugate the maleimide-activated PTD to the dsRNA:RND-DRBD complex.

200 μL of each of the reaction mixtures was diluted in 800 μL serum free media to generate the final concentrations of 0.9 μM siRNA:1.8 μM (RND-DRBD)-PTD or of 1.8 μM siRNA:3.6 μM (RND-DRBD)-PTD shown. These mixtures were added to 6×10³ human chondrocytes and the transfection was allowed to proceed as described in Example 1. GADPH expression in each of the reactions was determined using the method described in Example 1, and expressed as % remaining expression. The same transfection reactions were used in a parallel set of experiments to determine cellular toxicity of the mixtures using a commercially available WST-1 cell proliferation kit (Roche) which measures the metabolic activity of cells according to the manufacturer's instructions. The data are presented in FIG. 15.

The data in this example demonstrate that biologically active nucleic acid delivery complexes can be formed by including one or more PTDs to stable, inactive dsRNA:RND-DRBD complexes. The data also demonstrate that the addition of PTDs to the dsRNA:RND-DRBD complexes to form biologically active nucleic acid delivery complexes can be achieved without significant cytotoxic effects (as are observed for free PTDs when added directly and non-covalently to dsRNA).

Although a number of embodiments and features have been described above, it will be understood by those skilled in the art that modifications and variations of the described embodiments and features may be made without departing from the spirit of the invention as defined by the appended claims.

1. A nucleic acid delivery complex, comprising:
a double stranded RNA (dsRNA);
an RNA neutralization domain (RND);
a double-stranded RNA binding domain (DRBD) covalently attached to the RND to form an RND-DRBD chimera;
wherein the RND and DRBD of the RND-DRBD chimera bind cooperatively and non-covalently to the dsRNA to form a strong, tightly bound dsRNA:RND-DRBD complex, wherein the complex is in a stable, low energy state; and
a PTD covalently attached to the dsRNA:RND-DRBD complex, such that the PTD is free to interact with cell membrane to facilitate transport of the complex into a cell.

2. The nucleic acid delivery complex of claim 1, wherein the dsRNA is siRNA.

3. The nucleic acid delivery complex of claim 1, wherein the RND-DRBD and dsRNA of the strong, tightly bound dsRNA:RND-DRBD complex have a Kₐ of 1×10⁻¹° or greater or a Kₐ of 1×10⁻⁹ or greater, or a Kₐ of between about 1×10⁻¹° and 1×10⁻⁶.

4-6. (canceled)

7. The nucleic acid delivery complex of claim 1, wherein the RND-DRBD chimera is a recombinant protein.

8. The nucleic acid delivery complex of claim 7, wherein the recombinant protein further comprises at least one PTD.

9. The nucleic acid delivery complex of claim 1, wherein at least one PTD is covalently bound to the DRBD domain or the RND domain.

10-14. (canceled)

15. The method of claim 15, in which the contacting step is performed prior to the attaching step.

16-25. (canceled)

26. The method of claim 15, wherein the dsRNA is siRNA.

27. The method of claim 15, wherein the RND-DRBD and dsRNA of the strong, tightly bound dsRNA:RND-DRBD complex have a Kₐ of 1×10⁻¹⁰ or greater or a Kₐ of 1×10⁻⁹ or greater, or a Kₐ of between about 1×10⁻¹⁰ and 1×10⁻⁶.

28-56. (canceled)

57. A method for treating a subject with an RNA drug, comprising:
providing the nucleic acid delivery complex of any one of claims 1-14, and
administering the nucleic acid delivery complex to a subject such that the nucleic acid delivery complex undergoes PTD-mediated intracellular delivery of the dsRNA, after which the RNA drug exerts a desired pharmacological effect.

58-62. (canceled)

63. A pharmaceutical composition comprising:
the nucleic acid delivery complex of claim 1; and
one or more pharmaceutically acceptable excipients.

64. The pharmaceutical composition of claim 64, wherein the nucleic acid delivery complex forms particles with a diameter of less than 300 nm.

65. The pharmaceutical composition of claim 64, wherein the nucleic acid delivery complex forms particles with a diameter of less than 200 nm.

66-89. (canceled)
